SEQUENCE LISTING (12 sequences)

SEQ ID NO: 1 - DNA, 34796 bp, adenovirus serotype 35
catcatcaataatataccttatagatggaatggtgccaatatgtaaatgaggtgattttaaaaagtgtgggccgtgtggtgattggctgtggggttaacggttaaaaggggcggcgcggccgtgggaaaatgacgttttatgggggtggagtttttttgcaagttgtcgcgggaaatgttacgcataaaaaggcttcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtatttatacctcagggtttgtgtcaagaggccactcttgagtgccagcgagaagagttttctcctctgcgccggcagtttaataataaaaaaatgagagatttgcgatttctgcctcaggaaataatctctgctgagactggaaatgaaatattggagcttgtggtgcacgccctgatgggagacgatccggagccacctgtgcagctttttgagcctcctacgcttcaggaactgtatgatttagaggtagagggatcggaggattctaatgaggaagctgtgaatggcttttttaccgattctatgcttttagctgctaatgaaggattagaattagatccgcctttggacactttcaatactccaggggtgattgtggaaagcggtacaggtgtaagaaaattacctgatttgagttccgtggactgtgatttgcactgctatgaagacgggtttcctccgagtgatgaggaggaccatgaaaaggagcagtccatgcagactgcagcgggtgagggagtgaaggctgccaatgttggttttcagttggattgcccggagcttctggacatggctgtaagtcttgtgaatttcacaggaaaaatactggagtaaaggaactgttatgttcgcttttgttatatgaaaacccactgccactttatttacagtaaagtgtgtttaagttaaaatttaaaggaatatgctgtttttcacatgtatattgagtgtgagttttgtgcttcttattataagtcctgtgtctgatgctgatgaatcaccatctcctgattctactacctcacctcctgatattcaagcacctgttcctgtggacgtgcgcaagcccattcctgtgaagcttaagcctgggaaacgtccagcagtggagaaacttgaggacttgttacagggtggggacggacctttggacttgagtacacggaaacgtccaagacaataagtgttccatatccgtgtttacttaaggtgacgtcaatatttgtgtgagagtgcaatgtaataaaaatatgttaactgttcactggtttttattgctttttgggcggggactcaggtatataagtagaagcagacctgtgtggttagctcataggagctggctttcatccatggaggtttgggccattttggaagaccttaggaagactaggcaactgttagagagcgcttcggacggagtctccggtttttggagattctggttcgctagtgaattagctagggtagtttttaggataaaacaggactataaacaagaatttgaaaagttgttggtagattgcccaggactttttgaagctcttaatttgggccatcaggttcactttaaagaaaaagttttatcagttttagacttttcaaccccaggtagaactgctgctgctgtggcttttcttacttttatattagataaatggatcccgcagactcatttcagcaggggatacgttttggatttcatagccacagcattgtggagaacatggaaggttcgcaagatgaggacaatcttaggttactggccagtgcagcctttgggtgtagcgggaatcctgaggcatccaccggtcatgccagcggttctggaggaggaacagcaagaggacaacccgagagccggcctggaccctccagtggaggaggcggagtagctgacttgtctcctgaactgcaacgggtgcttactggatctacgtccactggacgggataggggcgttaagagggagagggcatccagtggtactgatgctagatctgagttggctttaagtttaatgagtcgcagacgtcctgaaaccatttggtggcatgaggttcagaaagagggaagggatgaagtttctgtattgcaggagaaatattcactggaacaggtgaaaacatgttggttggagccagaggatgattgggaggtggccattaaaaattatgccaagatagctttgaggcctgataaacagtataagatcagtagacggattaatatccggaatgcttgttacatatctggaaatggggctgaggtggtaatagatactcaagacaagacagttattagatgctgcatgatggatatgtggcctggagtagtcggtatggaagcagtcacttttgtaaatgttaagtttaggggagatggttataatggaatagtgtttatggccaataccaaacttatattgcatggttgtagcttttttggtttcaacaatacctgtgtagatgcctggggacaggttagtgtacgggggtgtagtttctatgcgtgttggattgccacagctggcagaaccaagagtcaattgtctctgaagaaatgcatattccaaagatgtaacctgggcattctgaatgaaggcgaagcaagggtccgtcactgcgcttctacagatactggatgttttattttaattaagggaaatgccagcgtaaagcataacatgatttgtggtgcttccgatgagaggccttatcaaatgctcacttgtgctggtgggcattgtaatatgctggctactgtgcatattgtttcccatcaacgcaaaaaatggcctgtttttgatcacaatgtgttgaccaagtgcaccatgcatgcaggtgggcgtagaggaatgtttatgccttaccagtgtaacatgaatcatgtgaaagtgttgttggaaccagatgccttttccagaatgagcctaacaggaatctttgacatgaacacgcaaatctggaagatcctgaggtatgatgatacgagatcgagggtgcgcgcatgcgaatgcggaggcaagcatgccaggttccagccggtgtgtgtagatgtgaccgaagatctcagaccggatcatttggttattgcccgcactggagcagagttcggatccagtggagaagaaactgactaaggtgagtattgggaaaactttggggtgggattttcagatggacagattgagtaaaaatttgttttttctgtcttgcagctgacatgagtggaaatgcttcttttaaggggggagtcttcagcccttatctgacagggcgtctcccatcctgggcaggagttcgtcagaatgttatgggatctactgtggatggaagacccgttcaacccgccaattcttcaacgctgacctatgctactttaagttcttcacctttggacgcagctgcagccgctgccgccgcctctgtcgccgctaacactgtgcttggaatgggttactatggaagcatcgtggctaattccacttcctctaataacccttctacactgactcaggacaagttacttgtccttttggcccagctggaggctttgacccaacgtctgggtgaactttctcagcaggtggccgagttgcgagtacaaactgagtctgctgtcggcacggcaaagtctaaataaaaaaaattccagaatcaatgaataaataaacgagcttgttgttgatttaaaatcaagtgtttttatttcatttttcgcgcacggtatgccctggaccaccgatctcgatcattgagaactcggtggattttttccagaatcctatagaggtgggattgaatgtttagatacatgggcattaggccgtctttggggtggagatagctccattgaagggattcatgctccggggtagtgttgtaaatcacccagtcataacaaggtcgcagtgcatggtgttgcacaatatcttttagaagtaggctgattgccacagataagcccttggtgtaggtgtttacaaaccggttgagctgggaggggtgcattcgaggtgaaattatgtgcattttggattggatttttaagttggcaatattgccgccaagatcccgtcttgggttcatgttatgaaggactaccaagacggtgtatccggtacatttaggaaatttatcgtgcagcttggatggaaaagcgtggaaaaatttggagacacccttgtgtcctccgagattttccatgcactcatccatgataatagcaatggggccgtgggcagcggcgcgggcaaacacgttccgtgggtctgacacatcatagttatgttcctgagttaaatcatcataagccattttaatgaatttggggcggagcgtaccagattggggtatgaatgttccttcgggccccggagcatagttcccctcacagatttgcatttcccaagctttcagttctgagggtggaatcatgtccacctggggggctatgaagaacaccgtttcgggggcgggggtgattagttgggatgatagcaagtttctgagcaattgagatttgccacatccggtggggccataaataattccgattacaggttgcaggtggtagtttagggaacggcaactgccgtcttctcgaagcaagggggccacctcgttcatcatttcccttacatgcatattttcccgcaccaaatccattaggaggcgctctcctcctagtgatagaagttcttgtagtgaggaaaagtttttcagcggttttagaccgtcagccatgggcattttggaaagagtttgctgcaaaagttctagtctgttccacagttcagtgatgtgttctatggcatctcgatccagcagacctcctcgtttcgcgggtttggacggctcctggagtagggtatgagacgatgggcgtccagcgctgccagggttcggtccttccagggtctcagtgttcgagtcagggttgtttccgtcacagtgaaggggtgtgcgcctgcttgggcgcttgccagggtgcgcttcagactcattctgctggtggagaacttctgtcgcttggcgccctgtatgtcggccaagtagcagtttaccatgagttcgtagttgagcgcctcggctgcgtggcctttggcgcggagcttacctttggaagttttcttgcataccgggcagtataggcatttcagcgcatacagcttgggcgcaaggaaaatggattctggggagtatgcatccgcgccgcaggaggcgcaaacagtttcacattccaccagccaggttaaatccggttcattggggtcaaaaacaagttttccgccatattttttgatgcgtttcttacctttggtctccataagttcgtgtcctcgttgagtgacaaacaggctgtccgtatctccgtagactgattttacaggcctcttctccagtggagtgcctcggtcttcttcgtacaggaactctgaccactctgatacaaaggcgcgcgtccaggccagcacaaaggaggctatgtgggaggggtagcgatcgttgtcaaccagggggtccaccttttccaaagtatgcaaacacatgtcaccctcttcaacatccaggaatgtgattggcttgtaggtgtatttcacgtgacctggggtccccgctgggggggtataaaagggggcggttctttgctcttcctcactgtcttccggatcgctgtccaggaacgtcagctgttggggtaggtattccctctcgaaggcgggcatgacctctgcactcaggttgtcagtttctaagaacgaggaggatttgatattgacagtgccggttgagatgcctttcatgaggttttcgtccatttggtcagaaaacacaatttttttattgtcaagtttggtggcaaatgatccatacagggcgttggataaaagtttggcaatggatcgcatggtttggttcttttccttgtccgcgcgctctttggcggcgatgttgagttggacatactcgcgtgccaggcacttccattcggggaagatagttgttaattcatctggcacgattctcacttgccaccctcgattatgcaaggtaattaaatccacactggtggccacctcgcctcgaaggggttcattggtccaacagagcctacctcctttcctagaacagaaagggggaagtgggtctagcataagttcatcgggagggtctgcatccatggtaaagattcccggaagtaaatccttatcaaaatagctgatgggagtggggtcatctaaggccatttgccattctcgagctgccagtgcgcgctcatatgggttaaggggactgccccagggcatgggatgggtgagagcagaggcatacatgccacagatgtcatagacgtagatgggatcctcaaagatgcctatgtaggttggatagcatcgcccccctctgatacttgctcgcacatagtcatatagttcatgtgatggcgctagcagccccggacccaagttggtgcgattgggtttttctgttctgtagacgatctggcgaaagatggcgtgagaattggaagagatggtgggtctttgaaaaatgttgaaatgggcatgaggtagacctacagagtctctgacaaagtgggcataagattcttgaagcttggttaccagttcggcggtgacaagtacgtctagggcgcagtagtcaagtgtttcttgaatgatgtcataacctggttggtttttcttttcccacagttcgcggttgagaaggtattcttcgcgatccttccagtactcttctagcggaaacccgtctttgtctgcacggtaagatcctagcatgtagaactgattaactgccttgtaagggcagcagcccttctctacgggtagagagtatgcttgagcagcttttcgtagcgaagcgtgagtaagggcaaaggtgtctctgaccatgactttgagaaattggtatttgaagtccatgtcgtcacaggctccctgttcccagagttggaagtctacccgtttcttgtaggcggggttgggcaaagcgaaagtaacatcattgaagagaatcttaccggctctgggcataaaattgcgagtgatgcggaaaggctgtggtacttccgctcgattgttgatcacctgggcagctaggacgatttcgtcgaaaccgttgatgttgtgtcctacgatgtataattctatgaaacgcggcgtgcctctgacgtgaggtagcttactgagctcatcaaaggttaggtctgtggggtcagataaggcgtagtgttcgagagcccattcgtgcaggtgaggatttgcatgtaggaatgatgaccaaagatctaccgccagtgctgtttgtaactggtcccgatactgacgaaaatgccggccaattgccattttttctggagtgacacagtagaaggttctggggtcttgttgccatcgatcccacttgagtttaatggctagatcgtgggccatgttgacgagacgctcttctcctgagagtttcatgaccagcatgaaaggaactagttgtttgccaaaggatcccatccaggtgtaagtttccacatcgtaggtcaggaagagtctttctgtgcgaggatgagagccgatcgggaagaactggatttcctgccaccagttggaggattggctgttgatgtgatggaagtagaagtttctgcggcgcgccgagcattcgtgtttgtgcttgtacagacggccgcagtagtcgcagcgttgcacgggttgtatctcgtgaatgagctgtacctggcttcccttgacgagaaatttcagtgggaagccgaggcctggcgattgtatctcgtgctcttctatattcgctgtatcggcctgttcatcttctgtttcgatggtggtcatgctgacgagcccccgcgggaggcaagtccagacctcggcgcgggaggggcggagctgaaggacgagagcgcgcaggctggagctgtccagagtcctgagacgctgcggactcaggttagtaggtagggacagaagattaacttgcatgatcttttccagggcgtgcgggaggttcagatggtacttgatttccacaggttcgtttgtagagacgtcaatggcttgcagggttccgtgtcctttgggcgccactaccgtacctttgttttttcttttgatcggtggtggctctcttgcttcttgcatgctcagaagcggtgacggggacgcgcgccgggcggcagcggttgttccggacccgggggcatggctggtagtggcacgtcggcgccgcgcacgggcaggttctggtattgcgctctgagaagacttgcgtgcgccaccacgcgtcgattgacgtcttgtatctgacgtctctgggtgaaagctaccggccccgtgagcttgaacctgaaagagagttcaacagaatcaatttcggtatcgttaacggcagcttgtctcagtatttcttgtacgtcaccagagttgtcctggtaggcgatctccgccatgaactgctcgatttcttcctcctgaagatctccgcgacccgctctttcgacggtggccgcgaggtcattggagatacggcccatgagttgggagaatgcattcatgcccgcctcgttccagacgcggctgtaaaccacggccccctcggagtctcttgcgcgcatcaccacctgagcgaggttaagctccacgtgtctggtgaagaccgcatagttgcataggcgctgaaaaaggtagttgagtgtggtggcaatgtgttcggcgacgaagaaatacatgatccatcgtctcagcggcatttcgctaacatcgcccagagcttccaagcgctccatggcctcgtagaagtccacggcaaaattaaaaaactgggagtttcgcgcggacacggtcaattcctcctcgagaagacggatgagttcggctatggtggcccgtacttcgcgttcgaaggctcccgggatctcttcttcctcttctatctcttcttccactaacatctcttcttcgtcttcaggcgggggcggagggggcacgcggcgacgtcgacggcgcacgggcaaacggtcgatgaatcgttcaatgacctctccgcggcggcggcgcatggtttcagtgacggcgcggccgttctcgcgcggtcgcagagtaaaaacaccgccgcgcatctccttaaagtggtgactgggaggttctccgtttgggagggagagggcgctgattatacattttattaattggcccgtagggactgcgcgcagagatctgatcgtgtcaagatccacgggatctgaaaacctttcgacgaaagcgtctaaccagtcacagtcacaaggtaggctgagtacggcttcttgtgggcgggggtggttatgtgttcggtctgggtcttctgtttcttcttcatctcgggaaggtgagacgatgctgctggtgatgaaattaaagtaggcagttctaagacggcggatggtggcgaggagcaccaggtctttgggtccggcttgctggatacgcaggcgattggccattccccaagcattatcctgacatctagcaagatctttgtagtagtcttgcatgagccgttctacgggcacttcttcctcacccgttctgccatgcatacgtgtgagtccaaatccgcgcattggttgtaccagtgccaagtcagctacgactctttcggcgaggatggcttgctgtacttgggtaagggtggcttgaaagtcatcaaaatccacaaagcggtggtaagcccctgtattaatggtgtaagcacagttggccatgactgaccagttaactgtctggtgaccagggcgcacgagctcggtgtatttaaggcgcgaataggcgcgggtgtcaaagatgtaatcgttgcaggtgcgcaccagatactggtaccctataagaaaatgcggcggtggttggcggtagagaggccatcgttctgtagctggagcgccaggggcgaggtcttccaacataaggcggtgatagccgtagatgtacctggacatccaggtgattcctgcggcggtagtagaagcccgaggaaactcgcgtacgcggttccaaatgttgcgtagcggcatgaagtagttcattgtaggcacggtttgaccagtgaggcgcgcgcagtcattgatgctctatagacacggagaaaatgaaagcgttcagcgactcgactccgtagcctggaggaacgtgaacgggttgggtcgcggtgtaccccggttcgagacttgtactcgagccggccggagccgcggctaacgtggtattggcactcccgtctcgacccagcctacaaaaatccaggatacggaatcgagtcgttttgctggtttccgaatggcagggaagtgagtcctattttttttttttttttgccgctcagatgcatcccgtgctgcgacagatgcgcccccaacaacagcccccctcgcagcagcagcagcagcagcaaccacaaaaggctgtccctgcaactactgcaactgccgccgtgagcggtgcgggacagcccgcctatgatctggacttggaagagggcgaaggactggcacgtctaggtgcgccttcgcccgagcggcatccgcgagttcaactgaaaaaagattctcgcgaggcgtatgtgccccaacagaacctatttagagacagaagcggcgaggagccggaggagatgcgagcttcccgctttaacgcgggtcgtgagctgcgtcacggtttggaccgaagacgagtgttgcgagacgaggatttcgaagttgatgaagtgacagggatcagtcctgccagggcacacgtggctgcagccaaccttgtatcggcttacgagcagacagtaaaggaagagcgtaacttccaaaagtcttttaataatcatgtgcgaaccctgattgcccgcgaagaagttacccttggtttgatgcatttgtgggatttgatggaagctatcattcagaaccctactagcaaacctctgaccgcccagctgtttctggtggtgcaacacagcagagacaatgaggctttcagagaggcgctgctgaacatcaccgaacccgaggggagatggttgtatgatcttatcaacattctacagagtatcatagtgcaggagcggagcctgggcctggccgagaaggtagctgccatcaattactcggttttgagcttgggaaaatattacgctcgcaaaatctacaagactccatacgttcccatagacaaggaggtgaagatagatgggttctacatgcgcatgacgctcaaggtcttgaccctgagcgatgatcttggggtgtatcgcaatgacagaatgcatcgcgcggttagcgccagcaggaggcgcgagttaagcgacagggaactgatgcacagtttgcaaagagctctgactggagctggaaccgagggtgagaattacttcgacatgggagctgacttgcagtggcagcctagtcgcagggctctgagcgccgcgacggcaggatgtgagcttccttacatagaagaggcggatgaaggcgaggaggaagagggcgagtacttggaagactgatggcacaacccgtgttttttgctagatggaacagcaagcaccggatcccgcaatgcgggcggcgctgcagagccagccgtccggcattaactcctcggacgattggacccaggccatgcaacgtatcatggcgttgacgactcgcaaccccgaagcctttagacagcaaccccaggccaaccgtctatcggccatcatggaagctgtagtgccttcccgatctaatcccactcatgagaaggtcctggccatcgtgaacgcgttggtggagaacaaagctattcgtccagatgaggccggactggtatacaacgctctcttagaacgcgtggctcgctacaacagtagcaatgtgcaaaccaatttggaccgtatgataacagatgtacgcgaagccgtgtctcagcgcgaaaggttccagcgtgatgccaacctgggttcgctggtggcgttaaatgctttcttgagtactcagcctgctaatgtgccgcgtggtcaacaggattatactaactttttaagtgctttgagactgatggtatcagaagtacctcagagcgaagtgtatcagtccggtcctgattacttctttcagactagcagacagggcttgcagacggtaaatctgagccaagcttttaaaaaccttaaaggtttgtggggagtgcatgccccggtaggagaaagagcaaccgtgtctagcttgttaactccgaactcccgcctgttattactgttggtagctcctttcaccgacagcggtagcatcgaccgtaattcctatttgggttacctactaaacctgtatcgcgaagccatagggcaaagtcaggtggacgagcagacctatcaagaaattacccaagtcagtcgcgctttgggacaggaagacactggcagtttggaagccactctgaacttcttgcttaccaatcggtctcaaaagatccctcctcaatatgctcttactgcggaggaggagaggatccttagatatgtgcagcagagcgtgggattgtttctgatgcaagagggggcaactccgactgcagcactggacatgacagcgcgaaatatggagcccagcatgtatgccagtaaccgacctttcattaacaaactgctggactacttgcacagagctgccgctatgaactctgattatttcaccaatgccatcttaaacccgcactggctgcccccacctggtttctacacgggcgaatatgacatgcccgaccctaatgacggatttctgtgggacgacgtggacagcgatgttttttcacctctttctgatcatcgcacgtggaaaaaggaaggcggtgatagaatgcattcttctgcatcgctgtccggggtcatgggtgctaccgcggctgagcccgagtctgcaagtccttttcctagtctacccttttctctacacagtgtacgtagcagcgaagtgggtagaataagtcgcccgagtttaatgggcgaagaggagtacctaaacgattccttgctcagaccggcaagagaaaaaaatttcccaaacaatggaatagaaagtttggtggataaaatgagtagatggaagacttatgctcaggatcacagagacgagcctgggatcatggggactacaagtagagcgagccgtagacgccagcgccatgacagacagaggggtcttgtgtgggacgatgaggattcggccgatgatagcagcgtgttggacttgggtgggagaggaaggggcaacccgtttgctcatttgcgccctcgcttgggtggtatgttgtgaaaaaaaataaaaaagaaaaactcaccaaggccatggcgacgagcgtacgttcgttcttctttattatctgtgtctagtataatgaggcgagtcgtgctaggcggagcggtggtgtatccggagggtcctcctccttcgtacgagagcgtgatgcagcagcagcaggcgacggcggtgatgcaatccccactggaggctccctttgtgcctccgcgatacctggcacctacggagggcagaaacagcattcgttactcggaactggcacctcagtacgataccaccaggttgtatctggtggacaacaagtcggcggacattgcttctctgaactatcagaatgaccacagcaacttcttgaccacggtggtgcagaacaatgactttacccctacggaagccagcacccagaccattaactttgatgaacgatcgcggtggggcggtcagctaaagaccatcatgcatactaacatgccaaacgtgaacgagtatatgtttagtaacaagttcaaagcgcgtgtgatggtgtccagaaaacctcccgacggtgctgcagttggggatacttatgatcacaagcaggatattttggaatatgagtggttcgagtttactttgccagaaggcaacttttcagttactatgactattgatttgatgaacaatgccatcatagataattacttgaaagtgggtagacagaatggagtgcttgaaagtgacattggtgttaagttcgacaccaggaacttcaagctgggatgggatcccgaaaccaagttgatcatgcctggagtgtatacgtatgaagccttccatcctgacattgtcttactgcctggctgcggagtggattttaccgagagtcgtttgagcaaccttcttggtatcagaaaaaaacagccatttcaagagggttttaagattttgtatgaagatttagaaggtggtaatattccggccctcttggatgtagatgcctatgagaacagtaagaaagaacaaaaagccaaaatagaagctgctacagctgctgcagaagctaaggcaaacatagttgccagcgactctacaagggttgctaacgctggagaggtcagaggagacaattttgcgccaacacctgttccgactgcagaatcattattggccgatgtgtctgatggaacggacgtgaaactcactattcaacctgtagaaaaagatagtaagaatagaagctataatgtgttggaagacaaaatcaacacagcctatcgcagttggtatctttcgtacaattatggcgatcccgaaaaaggagtgcgttcctggacattgctcaccacctcagatgtcacctgcggagcagagcaggtttactggtcgcttccagacatgatgaaggatcctgtcactttccgctccactagacaagtcagtaactaccctgtggtgggtgcagagcttatgcccgtcttctcaaagagcttctacaacgaacaagctgtgtactcccagcagctccgccagtccacctcgcttacgcacgtcttcaaccgctttcctgagaaccagattttaatccgtccgccggcgcccaccattaccaccgtcagtgaaaacgttcctgctctcacagatcacgggaccctgccgttgcgcagcagtatccggggagtccaacgtgtgaccgttactgacgccagacgccgcacctgtccctacgtgtacaaggcactgggcatagtcgcaccgcgcgtcctttcaagccgcactttctaaaaaaaaaatgtccattcttatctcgcccagtaataacaccggttggggtctgcgcgctccaagcaagatgtacggaggcgcacgcaaacgttctacccaacatcccgtgcgtgttcgcggacattttcgcgctccatggggtgccctcaagggccgcactcgcgttcgaaccaccgtcgatgatgtaatcgatcaggtggttgccgacgcccgtaattatactcctactgcgcctacatctactgtggatgcagttattgacagtgtagtggctgacgctcgcaactatgctcgacgtaagagccggcgaaggcgcattgccagacgccaccgagctaccactgccatgcgagccgcaagagctctgctacgaagagctagacgcgtggggcgaagagccatgcttagggcggccagacgtgcagcttcgggcgccagcgccggcaggtcccgcaggcaagcagccgctgtcgcagcggcgactattgccgacatggcccaatcgcgaagaggcaatgtatactgggtgcgtgacgctgccaccggtcaacgtgtacccgtgcgcacccgtccccctcgcacttagaagatactgagcagtctccgatgttgtgtcccagcggcgaggatgtccaagcgcaaatacaaggaagaaatgctgcaggttatcgcacctgaagtctacggccaaccgttgaaggatgaaaaaaaaccccgcaaaatcaagcgggttaaaaaggacaaaaaagaagaggaagatggcgatgatgggctggcggagtttgtgcgcgagtttgccccacggcgacgcgtgcaatggcgtgggcgcaaagttcgacatgtgttgagacctggaacttcggtggtctttacacccggcgagcgttcaagcgctacttttaagcgttcctatgatgaggtgtacggggatgatgatattcttgagcaggcggctgaccgattaggcgagtttgcttatggcaagcgtagtagaataacttccaaggatgagacagtgtcaatacccttggatcatggaaatcccacccctagtcttaaaccggtcactttgcagcaagtgttacccgtaactccgcgaacaggtgttaaacgcgaaggtgaagatttgtatcccactatgcaactgatggtacccaaacgccagaagttggaggacgttttggagaaagtaaaagtggatccagatattcaacctgaggttaaagtgagacccattaagcaggtagcgcctggtctgggggtacaaactgtagacattaagattcccactgaaagtatggaagtgcaaactgaacccgcaaagcctactgccacctccactgaagtgcaaacggatccatggatgcccatgcctattacaactgacgccgccggtcccactcgaagatcccgacgaaagtacggtccagcaagtctgttgatgcccaattatgttgtacacccatctattattcctactcctggttaccgaggcactcgctactatcgcagccgaaacagtacctcccgccgtcgccgcaagacacctgcaaatcgcagtcgtcgccgtagacgcacaagcaaaccgactcccggcgccctggtgcggcaagtgtaccgcaatggtagtgcggaacctttgacactgccgcgtgcgcgttaccatccgagtatcatcacttaatcaatgttgccgctgcctccttgcagatatggccctcacttgtcgccttcgcgttcccatcactggttaccgaggaagaaactcgcgccgtagaagagggatgttgggacgcggaatgcgacgctacaggcgacggcgtgctatccgcaagcaattgcggggtggttttttaccagccttaattccaattatcgctgctgcaattggcgcgataccaggcatagcttccgtggcggttcaggcctcgcaacgacattgacattggaaaaaaaacgtataaataaaaaaaaatacaatggactctgacactcctggtcctgtgactatgttttcttagagatggaagacatcaatttttcatccttggctccgcgacacggcacgaagccgtacatgggcacctggagcgacatcggcacgagccaactgaacgggggcgccttcaattggagcagtatctggagcgggcttaaaaattttggctcaaccataaaaacatacgggaacaaagcttggaacagcagtacaggacaggcgcttagaaataaacttaaagaccagaacttccaacaaaaagtagtcgatgggatagcttccggcatcaatggagtggtagatttggctaaccaggctgtgcagaaaaagataaacagtcgtttggacccgccgccagcaaccccaggtgaaatgcaagtggaggaagaaattcctccgccagaaaaacgaggcgacaagcgtccgcgtcccgatttggaagagacgctggtgacgcgcgtagatgaaccgccttcttatgaggaagcaacgaagcttggaatgcccaccactagaccgatagccccaatggccaccggggtgatgaaaccttctcagttgcatcgacccgtcaccttggatttgccccctccccctgctgctactgctgtacccgcttctaagcctgtcgctgccccgaaaccagtcgccgtagccaggtcacgtcccgggggcgctcctcgtccaaatgcgcactggcaaaatactctgaacagcatcgtgggtctaggcgtgcaaagtgtaaaacgccgtcgctgcttttaattaaatatggagtagcgcttaacttgcctatctgtgtatatgtgtcattacacgccgtcacagcagcagaggaaaaaaggaagaggtcgtgcgtcgacgctgagttactttcaagatggccaccccatcgatgctgccccaatgggcatacatgcacatcgccggacaggatgcttcggagtacctgagtccgggtctggtgcagttcgcccgcgccacagacacctacttcaatctgggaaataagtttagaaatcccaccgtagcgccgacccacgatgtgaccaccgaccgtagccagcggctcatgttgcgcttcgtgcccgttgaccgggaggacaatacatactcttacaaagtgcggtacaccctggccgtgggcgacaacagagtgctggatatggccagcacgttctttgacattaggggcgtgttggacagaggtcccagtttcaaaccctattctggtacggcttacaactctctggctcctaaaggcgctccaaatgcatctcaatggattgcaaaaggcgtaccaactgcagcagccgcaggcaatggtgaagaagaacatgaaacagaggagaaaactgctacttacacttttgccaatgctcctgtaaaagccgaggctcaaattacaaaagagggcttaccaataggtttggagatttcagctgaaaacgaatctaaacccatctatgcagataaactttatcagccagaacctcaagtgggagatgaaacttggactgacctagacggaaaaaccgaagagtatggaggcagggctctaaagcctactactaacatgaaaccctgttacgggtcctatgcgaagcctactaatttaaaaggtggtcaggcaaaaccgaaaaactcggaaccgtcgagtgaaaaaattgaatatgatattgacatggaattttttgataactcatcgcaaagaacaaacttcagtcctaaaattgtcatgtatgcagaaaatgtaggtttggaaacgccagacactcatgtagtgtacaaacctggaacagaagacacaagttccgaagctaatttgggacaacagtctatgcccaacagacccaactacattggcttcagagataactttattggactcatgtactataacagtactggtaacatgggggtgctggctggtcaagcgtctcagttaaatgcagtggttgacttgcaggacagaaacacagaactttcttaccaactcttgcttgactctctgggcgacagaaccagatactttagcatgtggaatcaggctgtggacagttatgatcctgatgtacgtgttattgaaaatcatggtgtggaagatgaacttcccaactattgttttccactggacggcataggtgttccaacaaccagttacaaatcaatagttccaaatggagaagataataataattggaaagaacctgaagtaaatggaacaagtgagatcggacagggtaatttgtttgccatggaaattaaccttcaagccaatctatggcgaagtttcctttattccaatgtggctctgtatctcccagactcgtacaaatacaccccgtccaatgtcactcttccagaaaacaaaaacacctacgactacatgaacgggcgggtggtgccgccatctctagtagacacctatgtgaacattggtgccaggtggtctctggatgccatggacaatgtcaacccattcaaccaccaccgtaacgctggcttgcgttaccgatctatgcttctgggtaacggacgttatgtgcctttccacatacaagtgcctcaaaaattcttcgctgttaaaaacctgctgcttctcccaggctcctacacttatgagtggaactttaggaaggatgtgaacatggttctacagagttccctcggtaacgacctgcgggtagatggcgccagcatcagtttcacgagcatcaacctctatgctacttttttccccatggctcacaacaccgcttccacccttgaagccatgctgcggaatgacaccaatgatcagtcattcaacgactacctatctgcagctaacatgctctaccccattcctgccaatgcaaccaatattcccatttccattccttctcgcaactgggcggctttcagaggctggtcatttaccagactgaaaaccaaagaaactccctctttggggtctggatttgacccctactttgtctattctggttctattccctacctggatggtaccttctacctgaaccacacttttaagaaggtttccatcatgtttgactcttcagtgagctggcctggaaatgacaggttactatctcctaacgaatttgaaataaagcgcactgtggatggcgaaggctacaacgtagcccaatgcaacatgaccaaagactggttcttggtacagatgctcgccaactacaacatcggctatcagggcttctacattccagaaggatacaaagatcgcatgtattcatttttcagaaacttccagcccatgagcaggcaggtggttgatgaggtcaattacaaagacttcaaggccgtcgccataccctaccaacacaacaactctggctttgtgggttacatggctccgaccatgcgccaaggtcaaccctatcccgctaactatccctatccactcattggaacaactgccgtaaatagtgttacgcagaaaaagttcttgtgtgacagaaccatgtggcgcataccgttctcgagcaacttcatgtctatgggggcccttacagacttgggacagaatatgctctatgccaactcagctcatgctctggacatgacctttgaggtggatcccatggatgagcccaccctgctttatcttctcttcgaagttttcgacgtggtcagagtgcatcagccacaccgcggcatcatcgaggcagtctacctgcgtacaccgttctcggccggtaacgctaccacgtaagaagcttcttgcttcttgcaaatagcagctgcaaccatggcctgcggatcccaaaacggctccagcgagcaagagctcagagccattgtccaagacctgggttgcggaccctattttttgggaacctacgataagcgcttcccggggttcatggcccccgataagctcgcctgtgccattgtaaatacggccggacgtgagacggggggagagcactggttggctttcggttggaacccacgttctaacacctgctacctttttgatccttttggattctcggatgatcgtctcaaacagatttaccagtttgaatatgagggtctcctgcgccgcagcgctcttgctaccaaggaccgctgtattacgctggaaaaatctacccagaccgtgcagggcccccgttctgccgcctgcggacttttctgctgcatgttccttcacgcctttgtgcactggcctgaccgtcccatggacggaaaccccaccatgaaattgctaactggagtgccaaacaacatgcttcattctcctaaagtccagcccaccctgtgtgacaatcaaaaagcactctaccattttcttaatacccattcgccttattttcgctctcatcgtacacacatcgaaagggccactgcgttcgaccgtatggatgttcaataatgactcatgtaaacaacgtgttcaataaacatcactttatttttttacatgtatcaaggctctggattacttatttatttacaagtcgaatgggttctgacgagaatcagaatgacccgcaggcagtgatacgttgcggaactgatacttgggttgccacttgaattcgggaatcaccaacttgggaaccggtatatcgggcaggatgtcactccacagctttctggtcagctgcaaagctccaagcaggtcaggagccgaaatcttgaaatcacaattaggaccagtgctctgagcgcgagagttgcggtacaccggattgcagcactgaaacaccatcagcgacggatgtctcacgcttgccagcacggtgggatctgcaatcatgcccacatccagatcttcagcattggcaatgctgaacggggtcatcttgcaggtctgcctacccatggcgggcacccaattaggcttgtggttgcaatcgcagtgcagggggatcagtatcatcttggcctgatcctgtctgattcctggatacacggctctcatgaaagcatcatattgcttgaaagcctgctgggctttactaccctcggtataaaacatcccgcaggacctgctcgaaaactggttagctgcacagccggcatcattcacacagcagcgggcgtcattgttggctatttgcaccacacttctgccccagcggttttgggtgattttggttcgctcgggattctcctttaaggctcgttgtccgttctcgctggccacatccatctcgataatctgctccttctgaatcataatattgccatgcaggcacttcagcttgccctcataatcattgcagccatgaggccacaacgcacagcctgtacattcccaattatggtgggcgatctgagaaaaagaatgtatcattccctgcagaaatcttcccatcatcgtgctcagtgtcttgtgactagtgaaagttaactggatgcctcggtgctcttcgtttacgtactggtgacagatgcgcttgtattgttcgtgttgctcaggcattagtttaaaacaggttctaagttcgttatccagcctgtacttctccatcagcagacacatcacttccatgcctttctcccaagcagacaccaggggcaagctaatcggattcttaacagtgcaggcagcagctcctttagccagagggtcatctttagcgatcttctcaatgcttcttttgccatccttctcaacgatgcgcacgggcgggtagctgaaacccactgctacaagttgcgcctcttctctttcttcttcgctgtcttgactgatgtcttgcatggggatatgtttggtcttccttggcttctttttggggggtatcggaggaggaggactgtcgctccgttccggagacagggaggattgtgacgtttcgctcaccattaccaactgactgtcggtagaagaacctgaccccacacggcgacaggtgtttttcttcgggggcagaggtggaggcgattgcgaagggctgcggtccgacctggaaggcggatgactggcagaaccccttccgcgttcgggggtgtgctccctgtggcggtcgcttaactgatttccttcgcggctggccattgtgttctcctaggcagagaaacaacagacatggaaactcagccattgctgtcaacatcgccacgagtgccatcacatctcgtcctcagcgacgaggaaaaggagcagagcttaagcattccaccgcccagtcctgccaccacctctaccctagaagataaggaggtcgacgcatctcatgacatgcagaataaaaaagcgaaagagtctgagacagacatcgagcaagacccgggctatgtgacaccggtggaacacgaggaagagttgaaacgctttctagagagagaggatgaaaactgcccaaaacagcgagcagataactatcaccaagatgctggaaatagggatcagaacaccgactacctcatagggcttgacggggaagacgcgctccttaaacatctagcaagacagtcgctcatagtcaaggatgcattattggacagaactgaagtgcccatcagtgtggaagagctcagctgcgcctacgagcttaaccttttttcacctcgtactccccccaaacgtcagccaaacggcacctgcgagccaaatcctcgcttaaacttttatccagcttttgctgtgccagaagtactggctacctatcacatcttttttaaaaatcaaaaaattccagtctcctgccgcgctaatcgcacccgcgccgatgccctactcaatctgggacctggttcacgcttacctgatatagcttccttggaagaggttccaaagatcttcgagggtctgggcaataatgagactcgggccgcaaatgctctgcaaaagggagaaaatggcatggatgagcatcacagcgttctggtggaattggaaggcgataatgccagactcgcagtactcaagcgaagcgtcgaggtcacacacttcgcatatcccgctgtcaacctgccccctaaagtcatgacggcggtcatggaccagttactcattaagcgcgcaagtcccctttcagaagacatgcatgacccagatgcctgtgatgagggtaaaccagtggtcagtgatgagcagctaacccgatggctgggcaccgactctccccgggatttggaagagcgtcgcaagcttatgatggccgtggtgctggttaccgtagaactagagtgtctccgacgtttctttaccgattcagaaaccttgcgcaaactcgaagagaatctgcactacacttttagacacggctttgtgcggcaggcatgcaagatatctaacgtggaactcaccaacctggtttcctacatgggtattctgcatgagaatcgcctaggacaaagcgtgctgcacagcacccttaagggggaagcccgccgtgattacatccgcgattgtgtctatctctacctgtgccacacgtggcaaaccggcatgggtgtatggcagcaatgtttagaagaacagaacttgaaagagcttgacaagctcttacagaaatctcttaaggttctgtggacagggttcgacgagcgcaccgtcgcttccgacctggcagacctcatcttcccagagcgtctcagggttactttgcgaaacggattgcctgactttatgagccagagcatgcttaacaattttcgctctttcatcctggaacgctccggtatcctgcccgccacctgctgcgcactgccctccgactttgtgcctctcacctaccgcgagtgccccccgccgctatggagtcactgctacctgttccgtctggccaactatctctcctaccactcggatgtgatcgaggatgtgagcggagacggcttgctggagtgccactgccgctgcaatctgtgcacgccccaccggtccctagcttgcaacccccagttgatgagcgaaacccagataataggcacctttgaattgcaaggccccagcagccaaggcgatgggtcttctcctgggcaaagtttaaaactgaccccgggactgtggacctccgcctacttgcgcaagtttgctccggaagattaccacccctatgaaatcaagttctatgaggaccaatcacagcctccaaaggccgaactttcggcttgcgtcatcacccagggggcaattctggcccaattgcaagccatccaaaaatcccgccaagaatttctactgaaaaagggtaagggggtctaccttgacccccagaccggcgaggaactcaacacaaggttccctcaggatgtcccaacgacgagaaaacaagaagttgaaggtgcagccgccgcccccagaagatatggaggaagattgggacagtcaggcagaggaggcggaggaggacagtctggaggacagtctggaggaagacagtttggaggaggaaaacgaggaggcagaggaggtggaagaagtaaccgccgacaaacagttatcctcggctgcggagacaagcaacagcgctaccatctccgctccgagtcgaggaacccggcggcgtcccagcagtagatgggacgagaccggacgcttcccgaacccaaccagcgcttccaagaccggtaagaaggatcggcagggatacaagtcctggcgggggcataagaatgccatcatctcctgcttgcatgagtgcgggggcaacatatccttcacgcggcgctacttgctattccaccatggggtgaactttccgcgcaatgttttgcattactaccgtcacctccacagcccctactatagccagcaaatcccgacagtctcgacagataaagacagcggcggcgacctccaacagaaaaccagcagcggcagttagaaaatacacaacaagtgcagcaacaggaggattaaagattacagccaacgagccagcgcaaacccgagagttaagaaatcggatctttccaaccctgtatgccatcttccagcagagtcggggtcaagagcaggaactgaaaataaaaaaccgatctctgcgttcgctcaccagaagttgtttgtatcacaagagcgaagatcaacttcagcgcactctcgaggacgccgaggctctcttcaacaagtactgcgcgctgactcttaaagagtaggcagcgaccgcgcttattcaaaaaaggcgggaattacatcatcctcgacatgagtaaagaaattcccacgccttacatgtggagttatcaaccccaaatgggattggcagcaggcgcctcccaggactactccacccgcatgaattggctcagcgccgggccttctatgatttctcgagttaatgatatacgcgcctaccgaaaccaaatacttttggaacagtcagctcttaccaccacgccccgccaacaccttaatcccagaaattggcccgccgccctagtgtaccaggaaagtcccgctcccaccactgtattacttcctcgagacgcccaggccgaagtccaaatgactaatgcaggtgcgcagttagctggcggctccaccctatgtcgtcacaggcctcggcataatataaaacgcctgatgatcagaggccgaggtatccagctcaacgacgagtcggtgagctctccgcttggtctacgaccagacggaatctttcagattgccggctgcgggagatcttccttcacccctcgtcaggctgttctgactttggaaagttcgtcttcgcaaccccgctcgggcggaatcgggaccgttcaatttgtagaggagtttactccctctgtctacttcaaccccttctccggatctcctgggcactacccggacgagttcataccgaacttcgacgcgattagcgagtcagtggacggctacgattgatgtctggtgacgcggctgagctatctcggctgcgacatctagaccactgccgccgctttcgctgctttgcccgggaacttattgagttcatctacttcgaactccccaaggatcaccctcaaggtccggcccacggagtgcggattactatcgaaggcaaaatagactctcgcctgcaacgaattttctcccagcggcccgtgctgatcgagcgagaccagggaaacaccacggtttccatctactgcatttgtaatcaccccggattgcatgaaagcctttgctgtcttatgtgtactgagtttaataaaaactgaattaagactctcctacggactgccgcttcttcaacccggattttacaaccagaagaacaaaacttttcctgtcgtccaggactctgttaacttcacctttcctactcacaaactagaagctcaacgactacaccgcttttccagaagcattttccctactaatactactttcaaaaccggaggtgagctccacggtctccctacagaaaacccttgggtggaagcgggccttgtagtactaggaattcttgcgggtgggcttgtgattattctttgctacctatacacaccttgcttcactttcctagtggtgttgtggtattggtttaaaaaatggggcccatactagtcttgcttgttttactttcgcttttggaaccgggttctgccaattacgatccatgtctagactttgacccagaaaactgcacacttacttttgcacccgacacaagccgcatctgtggagttcttattaagtgcggatgggaatgcaggtccgttgaaattacacacaataacaaaacctggaacaataccttatccaccacatgggagccaggagttcccgagtggtacactgtctctgtccgaggtcctgacggttccatccgcattagtaacaacactttcattttttctgaaatgtgcgatctggccatgttcatgagcaaacagtattctctatggcctcctagcaaggacaacatcgtaacgttctccattgcttattgcttgtgcgcttgccttcttactgctttactgtgcgtatgcatacacctgcttgtaaccactcgcatcaaaaacgccaataacaaagaaaaaatgccttaacctctttctgtttacagacatggcttctcttacatctctcatatttgtcagcattgtcactgccgctcacggacaaacagtcgtctctatcccactaggacataattacactctcataggacccccaatcacttcagaggtcatctggaccaaactgggaagcgttgattactttgatataatctgtaacaaaacaaaaccaataatagtaacttgcaacatacaaaatcttacattgattaatgttagcaaagtttacagcggttactattatggttatgacagatacagtagtcaatatagaaattacttggttcgtgttacccagttgaaaaccacgaaaatgccaaatatggcaaagattcgatccgatgacaattctctagaaacttttacatctcccaccacacccgacgaaaaaaacatcccagattcaatgattgcaattgttgcagcggtggcagtggtgatggcactaataataatatgcatgcttttatatgcttgtcgctacaaaaagtttcatcctaaaaaacaagatctcctactaaggcttaacatttaatttctttttatacagccatggtttccactaccacattccttatgcttactagtctcgcaactctgacttctgctcgctcacacctcactgtaactataggctcaaactgcacactaaaaggacctcaaggtggtcatgtcttttggtggagaatatatgacaatggatggtttacaaaaccatgtgaccaacctggtagatttttctgcaacggcagagacctaaccattatcaacgtgacagcaaatgacaaaggcttctattatggaaccgactataaaagtagtttagattataacattattgtactgccatctaccactccagcaccccgcacaactactttctctagcagcagtgtcgctaacaatacaatttccaatccaacctttgccgcgcttttaaaacgcactgtgaataattctacaacttcacatacaacaatttccacttcaacaatcagcatcatcgctgcagtgacaattggaatatctattcttgtttttaccataacctactacgcctgctgctatagaaaagacaaacataaaggtgatccattacttagatttgatatttaatttgttctttttttttatttacagtatggtgaacaccaatcatggtacctagaaatttcttcttcaccatactcatctgtgcttttaatgtttgcgctactttcacagcagtagccacagcaaccccagactgtataggagcatttgcttcctatgcactttttgcttttgttacttgcatctgcgtatgtagcatagtctgcctggttattaattttttccaacttctagactggatccttgtgcgaattgcctacctgcgccaccatcccgaataccgcaaccaaaatatcgcggcacttcttagactcatctaaaaccatgcaggctatactaccaatatttttgcttctattgcttccctacgctgtctcaaccccagctgcctatagtactccaccagaacaccttagaaaatgcaaattccaacaaccgtggtcatttcttgcttgctatcgagaaaaatcagaaatccccccaaatttaataatgattgctggaataattaatataatctgttgcaccataatttcatttttgatataccccctatttgattttggctggaatgctcccaatgcacatgatcatccacaagacccagaggaacacattcccccacaaaacatgcaacatccaatagcgctaatagattacgaaagtgaaccacaacccccactactccctgctattagttacttcaacctaaccggcggagatgactgaaacactcaccacctccaattccgccgaggatctgctcgatatggacggccgcgtctcagaacaacgacttgcccaactacgcatccgccagcagcaggaacgcgtggccaaagagctcagagatgtcatccaaattcaccaatgcaaaaaaggcatattctgtttggtaaaacaagccaagatatcctacgagatcaccgctactgaccatcgcctctcttacgaacttggcccccaacgacaaaaatttacctgcatggtgggaatcaaccccatagttatcacccaacaaagtggagatactaagggttgcattcactgctcctgcgattccatcgagtgcacctacaccctgctgaagaccctatgcggcctaagagacctgctaccaatgaattaaaaaaaaatgattaataaaaaatcacttacttgaaatcagcaataaggtctctgttgaaattttctcccagcagcacctcacttccctcttcccaactctggtattctaaaccccgttcagcggcatactttctccatactttaaaggggatgtcaaattttagctcctctcctgtacccacaatcttcatgtctttcttcccagatgaccaagagagtccggctcagtgactccttcaaccctgtctacccctatgaagatgaaagcacctcccaacacccctttataaacccagggtttatttccccaaatggcttcacacaaagcccagacggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaaaataaagtttaagtgtttttatttaaaatcacaaaattcgagtagttattttgcctccaccttcccatttgacagaatacaccaatctctccccacgcacagctttaaacatttggataccattagagatagacattgttttagattccacattccaaacagtttcagagcgagccaatctggggtcagtgatagataaaaatccatcgcgatagtcttttaaagcgctttcacagtccaactgctgcggatgcgactccggagtttggatcacggtcatctggaagaagaacgatgggaatcataatccgaaaacggtatcggacgattgtgtctcatcaaacccacaagcagccgctgtctgcgtcgctccgtgcgactgctgtttatgggatcagggtccacagtttcctgaagcatgattttaatagcccttaacatcaactttctggtgcgatgcgcgcagcaacgcattctgatttcactcaaatctttgcagtaggtacaacacattattacaatattgtttaataaaccataattaaaagcgctccagccaaaactcatatctgatataatcgcccctgcatgaccatcataccaaagtttaatataaattaaatgacgttccctcaaaaacacactacccacatacatgatctcttttggcatgtgcatattaacaatctgtctgtaccatggacaacgttggttaatcatgcaacccaatataaccttccggaaccacactgccaacaccgctcccccagccatgcattgaagtgaaccctgctgattacaatgacaatgaagaacccaattctctcgaccgtgaatcacttgagaatgaaaaatatctatagtggcacaacatagacataaatgcatgcatcttctcataatttttaactcctcaggatttagaaacatatcccagggaataggaagctcttgcagaacagtaaagctggcagaacaaggaagaccacgaacacaacttacactatgcatagtcatagtatcacaatctggcaacagcgggtggtcttcagtcatagaagctcgggtttcattttcctcacaacgtggtaactgggctctggtgtaagggtgatgtctggcgcatgatgtcgagcgtgcgcgcaaccttgtcataatggagttgcttcctgacattctcgtattttgtatagcaaaacgcggccctggcagaacacactcttcttcgccttctatcctgccgcttagcgtgttccgtgtgatagttcaagtacagccacactcttaagttggtcaaaagaatgctggcttcagttgtaatcaaaactccatcgcatctaattgttctgaggaaatcatccacggtagcatatgcaaatcccaaccaagcaatgcaactggattgcgtttcaagcaggagaggagagggaagagacggaagaaccatgttaatttttattccaaacgatctcgcagtacttcaaattgtagatcgcgcagatggcatctctcgcccccactgtgttggtgaaaaagcacagctaaatcaaaagaaatgcgattttcaaggtgctcaacggtggcttccaacaaagcctccacgcgcacatccaagaacaaaagaataccaaaagaaggagcattttctaactcctcaatcatcatattacattcctgcaccattcccagataattttcagctttccagccttgaattattcgtgtcagttcttgtggtaaatccaatccacacattacaaacaggtcccggagggcgccctccaccaccattcttaaacacaccctcataatgacaaaatatcttgctcctgtgtcacctgtagcgaattgagaatggcaacatcaattgacatgcccttggctctaagttcttctttaagttctagttgtaaaaactctctcatattatcaccaaactgcttagccagaagccccccgggaacaagagcaggggacgctacagtgcagtacaagcgcagacctccccaattggctccagcaaaaacaagattggaataagcatattgggaaccaccagtaatatcatcgaagttgctggaaatataatcaggcagagtttcttgtagaaattgaataaaagaaaaatttgccaaaaaaacattcaaaacctctgggatgcaaatgcaataggttaccgcgctgcgctccaacattgttagttttgaattagtctgcaaaaataaaaaaaaaacaagcgtcatatcatagtagcctgacgaacaggtggataaatcagtctttccatcacaagacaagccacagggtctccagctcgaccctcgtaaaacctgtcatcgtgattaaacaacagcaccgaaagttcctcgcggtgaccagcatgaataagtcttgatgaagcatacaatccagacatgttagcatcagttaaggagaaaaaacagccaacatagcctttgggtataattatgcttaatcgtaagtatagcaaagccacccctcgcggatacaaagtaaaaggcacaggagaataaaaaatataattatttctctgctgctgtttaggcaacgtcgcccccggtccctctaaatacacatacaaagcctcatcagccatggcttaccagagaaagtacagcgggcacacaaaccacaagctctaaagtcactctccaacctctccacaatatatatacacaagccctaaactgacgtaatgggactaaagtgtaaaaaatcccgccaaacccaacacacaccccgaaactgcgtcaccagggaaaagtacagtttcacttccgcaatcccaacaagcgtcacttcctctttctcacggtacgtcacatcccattaacttacaacgtcattttcccacggccgcgccgccccttttaaccgttaaccccacagccaatcaccacacggcccacactttttaaaatcacctcatttacatattggcaccattccatctataaggtatattattgatgatg

SEQ ID NO: 2 - DNA, 2550 bp, Artificial Sequence
  gag expression cassette
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgagatctaccatgggtgctagggcttctgtgctgtctggtggtgagctggacaagtgggagaagatcaggctgaggcctggtggcaagaagaagtacaagctaaagcacattgtgtgggcctccagggagctggagaggtttgctgtgaaccctggcctgctggagacctctgaggggtgcaggcagatcctgggccagctccagccctccctgcaaacaggctctgaggagctgaggtccctgtacaacacagtggctaccctgtactgtgtgcaccagaagattgatgtgaaggacaccaaggaggccctggagaagattgaggaggagcagaacaagtccaagaagaaggcccagcaggctgctgctggcacaggcaactccagccaggtgtcccagaactaccccattgtgcagaacctccagggccagatggtgcaccaggccatctccccccggaccctgaatgcctgggtgaaggtggtggaggagaaggccttctcccctgaggtgatccccatgttctctgccctgtctgagggtgccaccccccaggacctgaacaccatgctgaacacagtggggggccatcaggctgccatgcagatgctgaaggagaccatcaatgaggaggctgctgagtgggacaggctgcatcctgtgcacgctggccccattgcccccggccagatgagggagcccaggggctctgacattgctggcaccacctccaccctccaggagcagattggctggatgaccaacaacccccccatccctgtgggggaaatctacaagaggtggatcatcctgggcctgaacaagattgtgaggatgtactcccccacctccatcctggacatcaggcagggccccaaggagcccttcagggactatgtggacaggttctacaagaccctgagggctgagcaggcctcccaggaggtgaagaactggatgacagagaccctgctggtgcagaatgccaaccctgactgcaagaccatcctgaaggccctgggccctgctgccaccctggaggagatgatgacagcctgccagggggtggggggccctggtcacaaggccagggtgctggctgaggccatgtcccaggtgaccaactccgccaccatcatgatgcagaggggcaacttcaggaaccagaggaagacagtgaagtgcttcaactgtggcaaggtgggccacattgccaagaactgtagggcccccaggaagaagggctgctggaagtgtggcaaggagggccaccagatgaaggactgcaatgagaggcaggccaacttcctgggcaaaatctggccctcccacaagggcaggcctggcaacttcctccagtccaggcctgagcccacagcccctcccgaggagtccttcaggtttggggaggagaagaccacccccagccagaagcaggagcccattgacaaggagctgtaccccctggcctccctgaggtccctgtttggcaacgacccctcctcccagtaaaataaagcccgggcagatctgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctcta

SEQ ID NO: 3 - DNA, 2645 bp, Artificial Sequence
  SEAP expression cassette
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgagatcgatctaagtaagcttcctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctccgcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaacccgtggtccccgcgttgcttcctctgctggccgggacatcaggtggcccccgctgaattggaatcgatcagaattgatctgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctcta

SEQ ID NO: 4 - DNA, 49 bp, Artificial Sequence
  short synthetic polyA signal
aataaaagatctttattttcattagatctgtgtgttggttttttgtgtg

SEQ ID NO: 5 - DNA, 35167 bp, adenovirus serotype 24
catcatcaataatataccccacaaagtaaacaaaagttaacatgcaaatgagcttttgaatttagggcggggccagcgctgattggacgagagaagatgatgcaaatgacgtcacgacgcacggctaacggtcgccgcggaggcgtggcctagcccggaagcaagtcgcggggctgatgacgtataaaaaagcggactttagacccggaaacggccgattttcccgcggccacgcccggatatgaggtaattctgggcggatgcaagtaaaattaggtcattttggcgcgaaaactgaatgaggaagtgaaaagtgaaaaataccggtcccgcccagggcggaatatttaccgagggccgagagactttgaccgattacgtgggggtttcgattgcggtgttttttcgcgaatttccgcgtccgtgtcaaagtccggtgtttatgtcacagatcagctgatccacagggtatttaaaccagtcgagcccgtcaagaggccactcttgagtgccagcgagtagagatttctctgagctccgctcccagagtctgagaaaaatgagacacctgcgcctcctttcttcaactgtgcctattgacatggccgcattattgctggaggattatgtgagtacaatattggaggacgaactgcatccatctccatttgagctgggacctacacttcaggacctatatgatttggaggtagatgcccatgatgacgacccgaacgaagaggctgtgaatttaatatttccagaatctctgattcttcaggctgacatagccagcgaagctgtacctacaccacttcatacaccgactctgtcacccatacctgaattggaagaggaggacgagctagacctccgatgttatgaggaaggttttcctcccagcgattcagaggacgaacagggtgagcagagcatggctctaatctcaaaatatgcttgtgtggttgtggaagagcattttgtgttggacaatcctgaggtgcccgggcaaggctgtagatcctgccagtaccaccgggataagaccggagacacgaacgcctcctgcgctctgtgttacatgaaaaagaacttcagctttatttacagtaagtggagtgaatgtgagagagactgagtgcttaacacataactgggtaatgcttaaacagctgtgctaagtgtggtttatttttgtttctaggtccggtgtcagaggatgagtcatcaccctcagaagaagaccacccgtgtccccctgagctgtcaggcgaaacgcccctgcaagtgcacagacccaccccagtcagacccagtggcgagaggcgagcagctgttgaaaaaattgaggacttgttacatgacatgggtggggatgaacctttggacctgagcttgaaacgccccaggaactaggctcagctgtgcttagtcatgtgtaaataaagttgtacaataaaagtatatgtgacgcatgcaaggtgtggtttatgactcatgggcgtggcttagtcctatataagtggcaacacctgggcactggggcacagaccttcagggagttcctgatggatgtgtggactatccttgcagactttagcaagacacgccggcttgtagaggatagttcagacgggtgctccgggttctggagacactggtttggaactcctctatctcgtctggtgtacacagttaagaaggattataacgaggaatttgaaaatctttttgctgattgctctggcctgctagattctctaaatctcggccaccagtcccttttccaggaaagggtactccacagccttgatttttcaagcccagggcgcactacagccggggttgcttttgtggtttttctggttgacaaatggagccagaacacccaactgagcaggggctacattctggacttcgcagccatgcacctgtggagggcatgggtgaggcagcggggacagagaatcttgaactactggcttatacagccagcagctccgggtcttcttcgtctacacagacaaacatccatgttggaggaagaaatgaggcaggccatggacgagaacccgaggagcggcctggaccctccgtcggaagaggagctggattgaatcaggtatccagcctgtacccagagcttagcagggtgctgacatccatggccaggggagtgaagagggagaggagcgatgggggcaataccgggatgatgaccgagctgacggccagcctgatgaatcgcaagcgtccagagcgcattacctggcacgagctacagatggagtgtagggatgaggtgggcctgatgcaggataaatatggcctggagcagataaaaacccactggttgaacccagatgaggattgggaggaggccattaagaaatatgccaagatagccctgcgcccagattgcaagtacagggtgaccaagacggtgaatatcagacatgcctgctacatctcggggaacggggcagaggtggtcatcgataccctggacaaggccgccttcaggtgttgcatgatgggaatgagagccggagtgatgaatatgaattccatgattttcatgaacatgaagttcaatggagagaagtttaatggggtgatgttcatggccaacagtcacatgaccctgcacggctgcagtttcttcggcttcaacaatatgtgcgcagaggtctggggcgctgctaagatcaggggatgtaagttttatggctgctggatgggcgtggtcggaagacccaagagcgagatgtctgtgaagcagtgtgtgtttgagaaatgctacctgggagtctctaccgagggcaatgctagagtgagacattgctcttccctggagacgggctgcttctgcctggtgaagggcacagcctctctgaagcataatatggtgaagggctgcacggatgagcgcatgtacaacatgctgacatgcgactcgggggtctgccatatcctgaagaacatccatgtgacctcccacccccggaagaagtggccagtgtttgagaataacctactgatcaagtgccacatgcacctgggcgccagaaggggcaccttccagccgtaccagtgcaactttagccagaccaagctgctgctggagaacgatgccttctccagggtgaacctgaacggcatctttgacatggatgtctcggtgtacaagatcctgagatacgatgagaccaagtccagggtgcgcgcttgcgagtgcgggggcagacacaccaggatgcaaccagtggccctggatgtgaccgaggagctgaggcccgaccacctggtgatggcttgtaccgggaccgagttcagctccagtggggaggacacagattagaggtaggttgagtattagtgggcgtggctaaggtgactataaaggcgggtgtcttacgagggtctttttgcttttctgcagacatcatgaacgggactggcggggccttcgaaggggggctttttagcccttatttgacaacccgcctgccgggatgggccggagttcgtcagaatgtgatgggatcgacggtggacgggcgtccagtgcttccagcaaattcctcgaccatgacctacgcgaccgtggggaactcgtcgctcgacagcaccgccgcagccgcggcagccgcagccgccatgacagcgacgagactggcttcgagctacatgcccagcagcagcagtagcccctctgtgcccagttccatcatcgccgaggagaaactgctggccctgctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagaggtgggattggatgttgaggtacatgggcatgagcccgtcccgggggtggaggtagcaccactgcatggcctcgtgctctggggtcgtgttgtagatgatccagtcatagcaggggcgctgggcgtggtgctggatgatgtccttgaggaggagactgatggccacggggagccccttggtgtaggtgttggcgaagcggttgagctgggagggatgcatgcggggggagatgatgtggagtttggcctggatcttgaggttggcgatgttgccacccagatcccgcctggggttcatgttgtgcaggaccaccagaacggtgtagcccgtgcacttggggaacttgtcatgcaacttggaagggaatgcgtgaaagaatttggagacgcccttgtgcccacccaggttttccatgcactcatccatgatgatggcgatgggcccgtgggctgcggctttggcaaagacgtttctggggtcagagacatcgtaattatgctcctgggtgagatcatcataagacattttaatgaatttggggcggagggtgccagattgggggacaatggttccctcgggccccggggcgaagttcccctcacatatttgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggacatgcagctgccgtcgtcccggaggaggggggccacctcgttgagcatgtctctgacttggaggttttcccggacgagctcgccgaggaggcggtccccgcccagcgagagcagctcttgcagggaagcaaagtttttcaggggcttgagcccgtcggccatgggcatcttggcgagggtctgcgagaggagttcgaggcggtcccagagctcggtgacgtgctctacggcatctcgatccagcagacttcctcgtttcgggggttgggacgactgcgactgtagggcacgagacgatgggcgtccagcgctgccagcgtcatgtccttccagggtctcagtgtccgcgtgagcgtggtctccgtcacggtgaaggggtgggccccgggctgtgcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggtgcgagaaagacggactcgggggcgaaagcatccgctccgcagtgggcgcagacggtctcgcactcgaccagccaggtgagctcgggctgctcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcggaccactctgagacgaaggcgcgcgtccacgccaagacaaaggaggccacgtgcgaggggtagcggtcgttgtccaccagggggtccaccttttccacggtatgcagacacatgtccccctcctccgcatccaagaaggtgattggcttgtaggtgtaggccacgtgacccggggtccccgacgggggggtataaaagggggcgggtctgtgctcgtcctcactctcttccgcgtcgctgtccacgagcgccagctgttggggtaggtattccctttcgagagcgggcatgacctcggcactcaggttgtcagtttctagaaacgaggaggatttgatgttggcttgccctgccgcaatgctttttaggagactttcatccatctggtcagaaaagactatttttttattgtcaagcttggtggcgaaggagccatagagggcgttggagagaagcttggcgatggatctcatggtctgatttttgtcacggtcggctcgctccttggccgcgatgttgagctggacatactcgcgcgcgacgcacttccattcggggaagacggtggtgcgctcgtcgggcacgatcctgacgcgccagccgcggttatgcagggtgaccagatccacgctggtggccacctcgccgcgcaggggctcgttggtccagcagaggcgtccgcccttgcgcgagcagaacgggggcagcacatcaagcagatgctcgtcaggggggtccgcatcgatggtgaagatgcccggacagagttccttgtcaaaataatcgatttttgaggatgcatcatccaaggccatctgccactcgcgggcggccagcgctcgctcgtaggggttgaggggcggaccccagggcatgggatgcgtcagggcggaggcgtacatgccgcagatgtcgtagacatagatgggctccgagaggatgccgatgtaggtgggataacagcgccccccgcggatgctggcgcgcacgtagtcatacaactcgtgcgagggggccaagaaggcggggccgagattggtgcgctggggctgctcggcgcggaagacgatctggcgaaagatggcatgcgagttggaggagatggtgggccgttggaagatgttaaagtgggcatgaggcagacgaaccgagtcgcggatgaagtgcgcgtaggagtcttgcagcttggcgacgagctcggcggtgacgaggacgtccatggcgcagtagtccagcgtttcgcggatgatgtcataacccgcctctcctttcttctcccatagctcgcggttgagggcgtactcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcgaaggtatccctgaccatgactttcaagaactggtacttgaaatccgagtcgtcgcagccgccgtgctcccagagctcgaaatcggtgcgcttcttcgagagggggttaggcagagcgaaagtgacgtcattgaagagaatcttgcctgcccgcggcatgaaattgcgggtgatgcggaaagggcccgggacggaggctcggttgttgatgacctgggcggcgaggacgatctcgtcgaagccgttgatgttgtgcccgacgatgtagagttccatgaatcgcgggcggcctttaatgtgcggcagctttttgagctcctcgtaggtgaggtcctcggggcaatgcagtccgtgctgctcgagcgcccactcctggagatgtgggttggcttgcatgaatgaagcccagagctcgcgggccataagggtctggagctcgtcgcgaaagaggcggaactgctggcccacggccatcttttctggggtgacgcagtagaaagtaagggggtcccgctcccagcgatcccagcgtaagcgcacggctagatcgcgagcgagggcgaccagctctgggtcccccgagaatttcataaccagcataaaggggacgagctgcttgccgaaggaccccatccaggtgtaggtttctacatcgtaggtgacaaagagccgctccgtgcgaggatgagagccgattgggaagaactggatttcctgccaccagttggacgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgcgggagccaggtccagatctcggcgcggcgggggcggagagcgaagacgagggcgcgcagttgggagctgtccatggtgtcgcggagatccaggtccgggggcagggttctgaggttgacctcgtagaggcgggtgagggcgtgcttgagatgcagatggtacttgatctccacgggtgagttggtggctgtgtccacgcattgcatgagcccgtagctgcgcggggccacgaccgtgccgcggtgcgcttttagaagcggtgtcgcggacgcgctcccggcggcagcggcggttccggccccgcgggcaggggcggcagaggcacgtcggcgtggcgctcgggcaggtcccggtgctgcgccctgagagcgctggcgtgcgcgacgacgcggcggttgacatcctggatctgccgcctctgcgtgaagaccaccggccccgtgactttgaacctgaaagacagttcaacagaatcaatctcggcgtcattgacggcggcctgacgcaggatctcttgcacgtcgcccgagttgtcctggtaggcgatctcggacatgaactgctcgatctcctcctcctggagatcgccgcggcccgcgcgctccacggtggcggcgaggtcattggagatgcgacccatgagctgcgagaaggcgcccaggccgctctcattccagacgcggctgtagaccacgtccccgtcggcgtcgcgcgcgcgcatgaccacctgcgcgaggttgagctccacgtgccgcgtgaagacggcgtagttgcgcaggcgctggaagaggtagtttagggtggtggcgatgtgctcggtgacgaagaagtacatgatccagcggcgcaggggcatctcgctgatgtcgccgatggcctccagcctttccatggcctcgtagaaatccacagcgaagttgaaaaactgggcgttgcgggccgagaccgtgagctcgtcctccaggagcctgatgagttcggcgatggtggcgcgcacctcgcgctcgaaatccccgggggcctcctcctcttcctcttcttccatgacgacctcttcttctatttcttcctctgggggcggtggtggtggcggggcccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggcgggtccccgttgggcagcgagagggcgctgacgatgcatcttatcaattgcggtgtaggggacgtgagcgcgtcgagatcgaccggatcggagaatctttcgaggaaagcgtctagccaatcgcagtcgcaaggtaagctcaaacacgtagcagccctgtggacgctgttagaattgcggttgctgatgatgtaattgaagtaggcgtttttaaggcggcggatggtggcgaggaggaccaggtccttgggtcccgcttgctggatgcgaagccgctcggccatgccccaggcctggccctgacaccggctcaggttcttgtagtagtcatgcatgagcctctcaatgtcatcactggcggaggcggagtcttccatgcgggtgaccccgacgcccctgagcggctgcacgagcgccaggtcggcgacgacgcgctcggcgaggatggcctgttgcacgcgggtgagggtgtcctggaagtcgtccatgtcgacgaagcggtggtaggccccggtgttgatggtgtaggtgcagttggccatgagcgaccagttgacggtctgcaggccgggttgcacgacctctgagtacctgagccgcgagaaggcgcgcgagtcgaagacatagtcgttgcaggtgcgcacgaggtactggtatccaactaggaagtgcggcggcggctggcggtagagcggccagcgctgggtggccggcgcgcccggggccaggtcctcgagcatgaggcggtggtagccgtagaggtagcgggacatccaggtgatgccggcggcggtggtggaggcgcgcgggaactcgcggacgcggttccagatgttgcgcagcggcaggaaatagtccatggtcggcacggtctggccggtgagacgcgcgcagtcattgacgctctagaggcaaaaacgaaagcggttgagcgggctcttcctccgtagcctggcggaacgcaaacgggttaggccgcgtgtgtaccccggttcgagtcccctcgaatcaggctggagccgcgactaacgtggtattggcactcccgtctcgacccgagcccgatagccgccaggatacggcggagagccctttttgccgaccgaggggagtcgctagacttgaaagcggccgaaaaccccgccgggtagtggctcgcgcccgtagtctggagaagctttgccagggttgagtcgcggcagaacccggttcgcggacggccgcggcgagcgggacttggtcaccccgccgatttaaagacccacagccagccgacttctccagttacgggagcgagcccccttttttctttttgccagatgcatcccgtcctgcgccaaatgcgtcccaccccccctccggcgaccaccgcgaccgcggccgtagcaggcgccggcgctgtagccccgccacagcagacagagatggacttggaagagggcgaagggctggcgagactgggggcgccgtccccggagcgacacccccgcgtgcagctgcagaaggacgtgcgcccggcgtacgtgcctgcgcagaacctgttcagggaccgcagcggggaggagcccgaggagatgcgcgactgccgttttcgggcgggcagggagctgcgcgagggcctggaccgccagcgcgtgctgcgcgacgaggatttcgagccgaacgagcagacggggatcagccccgcgcgcgcgcacgtggcggcggccaacctggtgacggcctacgagcagacggtgaagcaggagcgcaacttccaaaagagtttcaacaaccatgtgcgcacgctaatcgcgcgcgaggaggtggccctgggcttgatgcacctgtgggacctggcggaggccatcgtgcagaacccggacagcaagcctctgacggcgcagctgttcctggtggtgcagcacagcagggacaacgaggcgttcagggaggcgctgctaaacatcgccgagcccgagggccgctggctgctggagctgatcaacatcttgcagagcatcgtagtgcaggagcgcagcctgagcctggccgagaaggtggcggctatcaactactcggtgctgagcctgggcaagttttacgcgcgcaagatttacaagacgccgtacgtgcccatagacaaggaggtgaagatagacagcttttacatgcgcatggcgctcaaggtgctgacgctgagcgacgacctgggcgtgtaccgcaacgaccgcatccacaaggccgtgagcgcgagccggcggcgcgagctgagcgaccgcgagctgatgctgagtctgcgccgggcgctggtagggggcgccgccggcggtgaggagtcctacttcgacatgggggcggacctgcattggcagccgagccggcgcgccttggaggccgcctacggtccagaggacttggatgaggatgaggaagaggaggaggatgcacccgctgcggggtactgacgcctccgtgatgtgtttttagatgcagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactctcggccattctggaggcggtggtcccctctcggaccaaccccacgcacgagaaggtgctggcgatcgtgaacgcgctggcggagaacaaggccatccgtcccgacgaggccgggctggtgtacaacgccctgctggagcgcgtgggccgctacaacagcacaaacgtgcagtccaacctggaccggctggtgacggacgtgcgcgaggccgtggcgcagcgcgagcggttcaagaacgagggcctgggctcgttggtggcgctgaacgccttcctggcgacgcagccggcgaacgtgccgcgcgggcaggacgattacaccaactttatcagcgcgctgcggctgatggtgaccgaggtgccccagagcgaggtgtaccagtcgggcccagactactttttccagacgagccggcagggcttgcagacggtgaacctaagccaggctttcaagaatctgcgcgggctgtggggcgtgcaggcgcccgtgggcgaccggtcgacggtgagcagcttgctaacgcccaactcgcggctgctgctgctgctgatcgcgcccttcaccgacagcggcagcgtgaaccgcaactcgtacctgggccacctgctgacgctttaccgcgaggccataggccaggcgcaggtggacgagcagaccttccaggagatcactagcgtgagccgcgcgctgggtcagaacgacaccgacagtctgagagccaccctgaacttcttgctgacaaatagacagcagaagattccggcgcagtacgcgctgtcggccgaggaggagcgcatcctgagatatgtgcagcagagcgtagggcttttcctgatgcaggagggggccacccccagcgccgcgctggacatgaccgcgcgcaacatggaacctagcatgtacgccgccaaccggccgttcatcaataagctgatggactacctgcaccgcgcggctgccatgaactcggactactttactaatgctatactaaacccgcactggctcccgccgccggggttctacacgggcgagtacgacatgcccgaccccaacgatgggttcctgtgggacgacgtggacagcgcggtgttctccccgaccttgcaaaagcgccaggaggcggtacgcacgcccgcgagcgagggcgcggtgggtcggagcccctttcctagcttagggagtttgcatagcttgccgggctcggtgaacagcggcagggtgagccggccgcgcttgctgggcgaggacgagtacctgaacgactcgctgctgcagccgccgcgggtcaagaacgccatggccaataacgggatagagagtctggtggacaaactgaaccgctggaagacctacgctcaggaccatagggatgcgcccgcgccgcggcgacagcgccacgaccggcagcggggcctggtgtgggacgacgaggactcggccgacgatagcagcgtgttggacttgggcgggagcggtggggccaacccgttcgcgcatctgcagcccagactggggcgacggatgttttgaatgaaataaaactcaccaaggccatagcgtgcgttctcttccttgttagagatgaggcgcgcggtggtgtcttcctctcctcctccctcgtacgagagcgtgatggcgcaggcaaccctggaggttccgtttgtgcctccgcggtatatggctcctacggagggcagaaacagcattcgttactcggaactggctccgcagtacgacaccactcgcgtgtacttggtggacaacaagtcggcggacatcgcttccctgaactaccaaaacgaccacagcaacttcctgaccacggtggtgcagaacaacgatttcacccccgccgaggccagcacgcagacgataaattttgacgagcggtcgcggtggggcggtgatttgaagaccattctgcacaccaacatgcccaatgtgaacgagtacatgttcaccagcaagtttaaggcgcgggtgatggtggctaggaaggtggtagatcagaatgataggagcaaggatgagttaaaatatgagtggtttgagtttaccctgcccgagggcaacttttccgagaccatgaccatagacctgatgaacaacgccatcttggaaaactacttgcaagtggggcggcaaaatggcgtgctggagagcgatatcggagtcaagtttgacagcaggaatttcaagctgggctgggacccggtaaccaagctggtgatgcctggggtctacacctacgaggccttccacccggacgttgtgctgctgccgggctgcggggtggacttcaccgagagccgcctgagcaacctcctgggcattcgcaagaagcaacctttccaagagggcttcaggatcatgtatgaggatctcgagggtggtaacatccccgccctcctggatgtcaagcaatatttggatagtaaaaagaagcttgaggaggcaacacagaatgcaaccagggctgctggagatatcagaggagacagtcatattccaagagctgtggaacaagcggctgaaaaggatctggtcattgtaccagtaacacaagatgaaagtaagagaagctataatgtcatagatggcacccatgacaccctctaccgaagttggtacctgtcctatacctacggggaccccgagaagggggtgcagtcgtggacgctgctcaccaccccggacgtcacctgcggcgcggagcaagtctactggtcgctgccggacctcatgcaagaccccgtcaccttccgctctacccagcaagtcagcaactaccccgtggttggcgccgagctcatgcccttccgcgccaagagcttttacaacgacctcgccgtctactcccagctcatccgcagctacacctccctcacccacgtcttcaaccgcttccccgacaaccagatcctctgccgtccgcccgcgcccaccatcaccacggtcagtgaaaacgtgcctgctctcacagatcacgggacgctaccgctgcgcagcagtatccgcggagtccagcgagtgaccgtcactgacgcccgtcgccgcacctgtccctacgtctacaaggccctgggcatagtcgcgccgcgcgtgctttccagtcgcaccttctaaaaaatgtctattctcatctcgcccagcaataacaccggctggggtcttactaggcccagcaccatgtacggaggagccaagaagcgctcccagcagcaccccgtccgcgtccgcggccacttccgcgctccctggggcgcttacaagcgcgggcggacttctaccgccgccgtgcgcaccaccgtcgacgacgtcatcgactcggtggtcgccgacgcgcgcaactatacccccgccccctccaccgtggacgcggtcatcgacagcgtggtggccgacgcgcgcgactatgccagacgcaagagccggcggcgacggatcgccaggcgccaccggagtacgcccgccatgcgcgccgcccgggctctgctgcgccgcgccagacgcacgggccgccgggccatgatgcgagccgcgcgccgcgccgccactgcaccccccgcaggcaggactcgcagacgagcggccgccgccgctgccgcggccatttctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggaccaccccaggcggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgtttacgcccggcgagcggtcctcggtcaggagcaagcgtagctatgacgaggtgtacggcgacgacgacatcctggaccaggcggcggagcgggcgggcgagttcgcctacgggaagcggtcgcgcgaagaggagctgatctcgttgccgctggacgagagcaaccccacgcctagcctgaagcccgtgaccctgcagcaggtgctgccccaagcagtgctgctgccgagccgcggggtcaagcgcgagggcgagaatatgtacccgaccatgcagatcatggtgcccaagcgccggcgcgtggaagaagtgctggacaccgtgaaaatggatgtggagcccgaggtcaaggtgcgccccatcaagcaggtggcgccgggcctgggcgtgcagaccgtggacattcagatccccaccgacatggatgttgacaaaaaaccctcgaccagcatcgaggtgcagaccgacccctggctcccagcctccaccgctgccgtctccacttctaccgccgccacggctaccgagcctcccagaaggcgaagatggggccctgccaaccggctgatgcccaactacgtattgcatccttccattatcccgacgccgggctatcgcggcacccggtactacgccagccgcaggcgcccagccagcaaacgccgccgccgcaccgccacccgccgccgtctggcccccgcccgcgtgcgccgcgtaaccacgcgccggggccgctcgctcgttctgcccaccgtgcgctaccaccccagcatcctttaatccgtgtgctgtgatactgttgcagagagatggctctcacttgccgcctgcgcatccccgtcccgaattaccgaggaagatcccgccgcaggagaggcatggcaggcagcggcctcaaccgccgccggcggcgggccatgcgcaggcgcctgagtggcggctttctgcccgcgctcatccccataatcgcggcggccatcggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcacggggcagttgttaagggaaaagctcaaagaccagaacttccagcagaaggtggtggacggcctagcctcgggcattaacggggtggtggacatagcaaaccaggccgtgcagcgcgagataaacagccgcctggacccgcggccgcccacggtggtggagatggaagatgcaactcctccgccgcccaagggcgagaagcggccgcggcccgacgcggaggagacgatcctgcaggtggacgagccgccctcgtacgaggaggccgtcaaggccggcatgcccaccacgcgtatcatcgcgccactggccactggtgtaatgaaacccgccacccttgacctgcctccgccacccacgcccgctccaccgaaggcagctccggttgtgcagccccctcctgtggcgaccgccgtgcgccgcgtccccgcccgccgccaggcccagaactggcagagcacgctgcacagtatcgtgggcctgggagtgaaaagtctgaagcgccgccgatgctattgagagagaggaaagaggacactaaagggagagcttaacttgtatgtgccttaccgccagagaacgcgcgaagatggctaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgctttgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcttacaacagcctggcccccaaaggcgcccccaactctagtcagtgggaacaagctaaagctaccaatgccggtcaaaaggaaactcacacatttggagtagccgctatgggcggagaagacattacagtgaaaggtcttcaaattggaactgatgaaactaaggaagatggagaggatgaaatttttgcagatcaaacattccagccagaacctcaagtgggagaacagaactggcaagaaacgtttgttttctatggaggcagagctcttaagaaagaaaccaaaatgaagccatgttatggctcttatgcgagacccacaaatgaaaagggaggacaggctaaatttacacttgatgaaaaaggtcagccaaccaaaattcctgatattacaatggatttctttgatagtccacaagatgatacatcaggtgtaactaataagccagatattgtcatgtatgcagaaaatgtaaatttagaagctcctgacacacatgtagtttacaaaccaggcaaagatgattctagttcttccgctaacctcacacaacaggccatgcctaacagaccgaactacatcgggttcagagacaactttgtgggtcttatgtactacaatagtactggcaacatgggtgtgctggctggtcaggcctctcagttgaatgctgtggtcgacttgcaagacagaaacaccgagctgtcttaccagctattgctagattctctgggtgacagaaccagatactttagcatgtggaattctgcagtggacagctatgaccccgatgtcaggatcattgagaatcacggtgtggaagatgaacttccaaactattgcttcccactgaatggcagtggttctaacagcacatacaaaggtgttaaagctggaactggaaacaattgggatgacgatgaaaatgttgcaagacaaaatcagattggcactggcaacctgttcgccatggagatcaacctccaggccaacctatggaagagttttctgtactcgaacgtggccctgtacctgcccgactcctacaagtacacgccggccaacgtcacgctgcccaccaacaccaacacctacgactacatgaacggccgcgtggtagccccctcgctggtggacgcctacatcaacattggcgcccgctggtcgctggaccccatggacaatgtcaatcccttcaaccaccaccgcaacgcgggcctgcgctaccgctccatgctcctgggcaacggccgctacgtgcccttccacatccaagtgccccaaaagttctttgccatcaagaacctgcttctgctccccggttcctacacctacgagtggaacttccgcaaggacgtcaacatgatcctgcagagttccctcggcaacgacctgcgcgtcgacggcgcctccgtccgcttcgacagcgtcaacctctacgccaccttcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggctggagtttcacccggctcaagaccaaggaaactccctccctcggctcgggtttcgacccctactttgtctactcgggctccatcccctacctcgacgggaccttctacctcaaccacaccttcaagaaggtctccatcatgttcgactcctcggtcagctggcccggcaacgaccggctgctcacgccgaacgagttcgagatcaagcgcagcgttgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctacccttccagcacaacaactcgggcttcaccggctaccttgcgcccaccatgcgccaggggcagccctaccccgccaacttcccctacccgctcatcggctccaccgcagttccctccgtcacccagaaaaagttcctctgcgacagggtcatgtggcgcatcccattctccagcaactttatgtccatgggcgccctcaccgacctgggtcagaacatgctctatgccaactcggcccacgcgctcgacatgacctttgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgctaccacttaagcatgagcggctccagcgaacaagagctcgcggccatcgtgcgcgacctgggatgcgggccctactttttgggaacccacgacaagcgcttccctggcttccttgccggcgacaagctggcctgcgccatcgtcaacacggccggccgcgagaccggaggcgtgcactggctcgcctttggctggaatccgcgctcgcgcacctgctacatgttcgacccctttgggttctcggaccgccggctcaagcagatttacagcttcgagtacgaggccatgctgcgccgaagcgcgcttgcctcctcgcccgaccgctgtctcagcctcgagcagtccacccagaccgtgcaggggcccgactccgccgcctgcggacttttttgttgcatgtttttgcatgccttcgtgcactggcccgaccgacccatggacggaaaccccaccatgaacttgctgacgggggtgccaaacggcatgctacaatcgccacaggtgctgcccaccctcaggcgcaaccaggaggagctctaccgcttcctcgcgcgccactccccttactttcgatcccaccgcgccgccatcgaaaacgccaccgcttttgataaaatgaaacaactgcgtgtatctcaataaacagcactttattttacatgcactggagtatatgcaagttatttaaaagtcgaaggggttctcgcgctcgtcgttgtgcgccgcgctggggagggccacgttgcggtactggtacttgggaagccacttgaactcggggatcaccagtttgggcactggggtctcggggaaggtctcgctccacatgcgccggctcatctgcagggcgcccagcatgtccgggccggagatcttgaaatcacaattggggccggtgctctgcgcgcgcgagttgcggtacacggggttgcagcactggaacaccattagactggggtacttcacactggcaagcacgctcttgtcgctgatctgatccttgtccaggtcctcggcgttgctcaggccgaacggggtcatcttgcacagctggcggcccaggaagggcacgctctgaggcttgtggttacactcgcagtgcacgggcatcagcatcatccccgcgccgcgctgcatattcgggtagagggccttgacgaaggccgtgatctgcttgaaagcttgctgggccttagccccctcgctgaaaaacaggccgcagctcttcccgctaaactggttattcccgcacccggcatcatgcacgcagcagcgcgcgtcatggctggtcagttgcaccacgctacgtccccagcggttctgggtcaccttggccttgctgggctgctccttcaacgcgcgctgcccgttctcgctggtcacatccatctccaccacgtggtccttgtggatcatcaccgtcccatgcagacacttgagctgaccctcgacatcgcagcagccatgatcccacagggcgcagccggtgcactcccagttcttatgcgcgatcccgctgtggctgaagatgtaaccttgcaacaggcgacccatgacggtgctaaatgctttctgggtggtgaaggtcagttgcagaccgcgggcctcctcgttcatccaggtctggcacatcttttggaagatctcggtctgctcgggcatgagcttgtaagcatcgcgcaggccgctgtcgacgcggtagcgttccatcagcacgttcatggtatccatgcccttctcccaggacgagaccagaggcagactcagggggttgcgcacgttcaggacaccgggggtcgcaggctcgacgatgcgttttccgtccttgccttccttcaacagaaccggaggctggctgaatcccactcccacgattacggcatcttcctggggcatctcttcgtcggggtctaccttggtcacatgcttggtctttctggcttgcttcttttttggagggctgtccacggggaccacgtcctcctcggaagacccggagcccacccgctgatactttcggcgcttggtgggcagaggaggtggtggcggcgaggggctcctctcctgctccggcggatagcgcgccgacccgtggccccggggcggagtggcctctcgctccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctagaacccccacaggatgaacaggagcacgagcaagacgcaggccaggaggagaccgacgctgggctccagcatggctacctgggaggagaggaggatgtgctgctaaaacacttgcagcgccaatccatcatcctccgggacgccctggccgaccggagcgaaacccctctcagcgtcgaggagctgtgtcgggcctacgagctcaacctcttctcgccgcgcgtgccccccaaacgccagcccaacggcacctgcgagcccaacccgcgtctcaacttctatcccgtctttgcggtccccgaggccctagccacctatcacatctttttcaagaaccaaaagatccccgtctcctgccgcgccaaccgcacccgcgccgacgcgctcctcgctctggggcccggcgcgcgcatacctgatatcgcttccctggaagaggtgcccaagatcttcgaagggctcggtcgggacgagacgcgcgcggcaaacgctctgaaagaaacagcagaggaagagggtcacactagcgccctggtagagttggaaggcgacaacgccaggctggccgtgctcaagcgcagcgtcgagctcacccacttcgcctaccccgccgtcaacctcccgcccaaggtcatgcgtcgcatcatggatcagctcatcatgccccacatcgaggccctcgatgaaagtcaggagcagcgccccgaggacgcccggcccgtggtcagcgacgagcagctcgcgcgttggctcgggacccgcgacccccaggctttggaacagcggcgcaagctcatgctggccgtggtcctggtcaccctcgagctcgaatgcatgcgccgcttcttcagcgaccccgagaccctgcgtaaggtcgaggagaccctgcactacactttcaggcacggtttcgtcaggcaggcctgcaagatctccaacgtggagctgaccaacctggtctcatgcctggggatcctgcacgagaaccgcctgggacagaccgtgctccactctactctgaagggcgaggcgcgtcgggactatgtccgcgactgtgtatttctctttatctgccacacctggcaagcagccatgggcgtgtggcagcagtgtctcgaggacgaaaatctgaaggagctggacaagcttcttgctagaaaccttaaaaagctgtggacgggcttcgacgagcgcaccgtcgcctcggacctggccgagatcgtttttccagaacgcctgaggcagacgctgaaaggcgggctgcccgacttcatgagccagagcatgttgcaaaactaccgcactttcattctcgagcgatctgggatgctacccgccacctgcaacgcattcccctccgactttgtcccgctgagctaccgcgagtgtcccccgccgctgtggagccactgctatctcttgcagctggccaactacatcgcctaccactcggacgtgatcgaggacgtgagcggcgaggggcttctcgagtgccactgccgctgcaacctgtgctccccgcaccgctccctggtctgcaacccccagcttctgagcgagacccaggtcatcggtaccttcgagctgcaaggtccgcaggagtccaccgctccgctgaaactcacgccggggttgtggacttccgcgtacctgcgcaaatttgtacccgaggactaccacgcccatgaaataaagttcttcgaggaccaatcgcgcccacagcacgcggatctcacggcctgcgtcatcacccagggcgcgatcctcgcccaattgcacgccatccaaaaatcccgccaagagtttcttctaaaaaagggtagaggggtctacctggacccccagacgggcgaggtgctcaacccgggtctcccccagcatgccgaggaagaagcaggagccgctagtggagcagatggaagaagaatgggacagccaggcagaggaggacgaatgggaggaggagacagaggaggaagaattggaagaggtggaagaggagcaggaaacagagcagcccgtcgccgcaccatccgcgccggcagccccgccggtcacggatacaacctccacagctccggccaagcctcctcgtagatgggatcgagtgaagggtgacggtaagcacgagcggcagggctaccgatcatggagggtccacaaagccgcgatcatcgcctgcttgcaagactgcggggggaacatcgctttcgcccgccgctacctgctcttccaccgcggggtgaacatcccccgcaacgtgttgcattactaccgtcaccttcacagctaagaaaaagcaagtaagaggagtcgccggaggaggcctgaggatcgcggcgaacgagccctcgaccaccagggagctgaggaaccggatcttccccactctttatgccatttttcagcagagtcgaggtcagcagcaagaactgaaagtaaaaaaccggtctctgcgctcgctcacccgcagttgcttgtaccacaaaaacgaagatcagctgcagcgcactctcgaagacgccgaggctctgttccacaagtactgcgcgctcactcttaaagactaaggcgcgcccacccggaaaaaaggcgggaattacctcatcgccaccatgagcaaggagattcccaccccttacatgtggagctatcagccccagatgggcctggccgcgggcgcctcccaggactactccacccgcatgaactggctcagtgccggcccctcgatgatctcacgggtcaacggggtccgtaaccatcgaaaccagatattgttggagcaggcggcggtcacctccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctggtgatccgaggcagaggcacacagctcaacgacgagttggtgagctcttcgatcggtctgcgaccggacggagtgttccaactagccggagccgggagatcgtccttcactcccaaccaggcctacctgaccttgcagagcagctcttcggagcctcgctccggaggcatcggaaccctccagttcgtggaggagtttgtgccctcggtctacttcaaccccttctcgggatcgccaggcctctacccggacgagtttataccgaacttcgacgcagtgagagaagcggtggacggctacgactgaatgtcccatggtgactcggctgagctcgctcggttgaggcatctggaccactgccgccgcctgcgctgcttcgcccgggagagctgcggactcatctactttgagtttcccgaggagcaccccaacggccctgcacacggagtgcggatcaccgtagagggcaccaccgagtctcacctggtcaggttcttcacccagcaacccttcctggtcgagcgggaccggggagctaccacctacaccgtctactgcatctgtcctaccccgaagttgcatgagaatttttgctgtactctttgtggtgagtttaataaaagctgaactaagaaccttctttggaatcccttgtcatcatcaaatcaacaagaccatcaacttcacctttgaggaacaggtgaactttacctgcaagccacacaagaagtacatcatctggttttatcacaacactactctagcagtagccaacacctgctcgaacgacggtgttctcctacctaacaatctcaccagtggactaaccttctcagttaaaagggcaaagctaattcttcatcgccctattgtagaaggaacttaccagtgtcagagcggaccttgcttccacagtttcactttggtgaacgttaccggcagcagcacagccgctccagaaacatctaaccttctttctgatactaacaaacctcgtgtcggaggtgagctttgggttccatctctaacagagggtgggagttctattgaagtggttgggtatttgattttaggggtggtcattggtgggtgcatagcagtgctgtatcaacttccttgctgggtcgaaatcagggtatttatctgctgggtcagacattgtggggaggaaccatgaaggggctcttgctgattatcctttccctggtggggggtgtgctgtcatgccacgaacagccacgatgtaacattaccacaggcaatgagaggaacgactgctctgtagttatcaaatgcgagcaccattgtcctctcaacatcacattcaagaataagaccatgggaaatgtatgggtgggattctggcaaccaggagatgagcagaactacacggtcactgtccatggtagcgatggcaatcacactttcggtttcaaattcatttttgaagtcatgtgtgatatcacactacatgtggctagacttcatggcttgtggccccctaccaaggagaacatggtgggtttttctttggcttttgtgatcatggcctgcttgatgtcaggtctgctggtaggggctctagtgtggtttctgaaacgcaagcccaggtacggaaatgaggagaaggaaaaattgctataaattctttttctcttcgcacaaccatgaatacagtgttccgtatcgtgctgctctctcttcttgtagctttcggtcaggcaggaattcatattattaatgctacatggtgggataatataactttagtgggaccctcagatactccagttacctggtatgatggcaagggattgcaattttgtgacggaagtacagttaagaatccgcagatcagacatacttgtaatgatcaaaacttaactctgattcatgttaacaaaacccatgaaagaacatacatgggttacagacatgacagtaagggaaaagtagactataaggttacagtcattccacctcctcctgctactgtaaagccacaaccagatccagaaaatgtctttgtttatatgggaaataatgtaactttagttggacctccaggaattccagttagttggtattatcataatggcacacagttctgcgatggagataaaattattcatccagaattcaaccacacctgtgataaacaaaaccttacactgctgtttgtaaactttacacatgatggaggctatcttggattcaattacaaaggtactcagagaattcagtatgaggttatagttttagatcgatttccaaattctggtcagatgaaaattgaagaacaaagtgaggaaacagaacagaaacatactgagcataataaggctggacaaaagcagggtatagatacaaatcaaaagaaagctaataacagacaaaagccatctcaaaggccatcaagaagacggccgacaaacactcctgagacaaaacaacttacagtgtctattgggtctaacttaactttagttggtccagatggaaaagtcacttggtatgatggtgatttaaaaagaccatgtgaagaacaaaactataggcttccacatcagtgtagtgctcagaacttaactttaattaatgtaactaaatctcatgagggaacttactatggcactaatgacaaagacgaaagcaaaagatacagagtgaaagtgaacactacaaattctcaagctgtaaaaattaacccatataccagacctactactcctgatcagaaacacagatttgaattacaaattgaaaataatgcaaatgatgaagaatcaaaaattccatctactactgtggcaatcgtggtgggagtgattgcgggcttcataactataatcattgtcattctgtgctacatctgctgccgcaagcgtcccagggcatacaatcatatggtagacccactactcagcttctcttactgagactcagtcactttcatttcagaaccatgaaggctttcacagcttgcgttctgtttaacataatcacacttagtgtagctgcaaatggttttaaacatgttaatgttaccagattaagtaatgtaacactgacaggagctggaattaatactacatggacagggtattttaatgagggtccaaaaggaaaaaatgggtggatgaatatttgcacatggggcgatcctagatatgtgtgccatggaaatagcagtactattactaatcttacagttgtggcacttctaaatttaaccactaacagaagatttaaagcagaaagttttactagtaacgatggttatgaaactaccagtgcaaaattttatgaaattaaaattattgagcttccaacaactagagcacccaccacagttaggacaacacagcctaccactgtgcccactacacatccaaccaccacagtcagtacaactattgagaccactactcatactacacagctagacacaacagtgcagaatactactttattgattgggtttttactgagaggaaatgaaagtactactgaacagacagaggctacctcaagtgccttcagcagcactgcaaatttaacttcgcttgcttggactaatgaaaccggagtatcattgatgaatcgacagccttactcaggtttggatattcaaattacttttctggttgtctgtgggatctttattcttgcggttcttctgtactttgtctgctgcaaagccagagagaaatctaggcggcccatatacaggccagtaatcggggaacctcagcctctccaagtggatggaggcttaaggaatcttctcttctcttttacagtatggtgatcagccatgattcctaggttcttcctatttaacatcctgttctgtctcttcaacatctgtgctgccttcgcggccgtctcgcacgcctcgcccgactgtctagggcctttcccaacatacctcctctttgccctgctaacctgcacctgcgtctgcagcattgtctgcgtggtcatcacctttctgcagctcatcgactggtgctgcgcgcgctacaattatctccaccacagtcccgaatacagggacgagaacgtagccagaatcttaaggctcatctgaccatgcagcctctgctcatgctgatatccctcctatcccctgcccttgccacttctgctgattactctaaatgcaaattcgcggacatatggaatttcttagattgctatcaggagaaaattgatatgccctcctattacttggtgattgttggggtagtcatggtctgctcatgcactttctttgccattatgatctacccctgttttaatcttggctggaactctgttgaggcattcacatacacactagaaaacagttcactagcctccacgccaccacccacaccgcctccccgcagaaatcagttccctatgattcagtacttagaagagccccctccccggcccccttccactgttagctactttcacataaccggcggcgatgactgaccacctggacctcgagatggacggccaggcctccgagcagcgcatcctgcaactgcgcgtccgacagcagcaggagcgggccgccaaggagctcctcgatgccatcaacatccaccagtgcaagaagggcatcttctgcctggtcaagcaggcaaagatcacctacgagctcgtgtccggcggcaagcagcatcgcctcgcctatgagctaccccagcagaagcaaaagttcacctgcatggtgggcgtcaaccccatagtcatcacccagcagtcgggcgagaccaacggctgcatccactgctcctgcgaaagccccgagtgcatctactccctcctcaagaccctttgcggactccgcgacctcctccccatgaactgatgttgattaaaagcccaaaaaccaatcaaacccttccccaattactcataagaataaatcattggaactaatcattcaataaagatcacttacttgaaatctgaaagtatgtctctggtgtagttgttcagcagcacctcggaaccctcctcccagctctggtactccagtccccggcgggcggcgaacttcctccacaccttgaaagggatgtcaaattcctggtccacaattttcattgtcttccctcagatgacaaagaggctccgggtggaagatgacttcaaccccgtctacccctatggctacgcgcggaatcagaatatccccttccttactcccccctttgtttcttccgatggattccaaaacttcccacctggggtcctgtcactcaaactggctgacccaatcgccatcactaatggggatgtttcactcaaggtgggagggggtcttactgttgaaaaagatagtggaaatctaaaggtgaaccctaaggctcccttgcaagttacaactgataaacagttggaaattgcactggcttatccatttgaagtcagtaatggcaagcttggcataaaagcaggtcatggattgaaagtcattgacaaaattgctggtttggaaggtttggcaggtacgcttgtagttttgactggaaaaggaataggtactgaaaatcttgaaaacagtgatgggtcaagtagaggagttggtataaacgtaagacttgctaaagatggaggtctgtcttttgataaaaagggtgatttagttgcttggaataaacatgatgacagacgcactctatggacaactcccgacccatccccaaattgtacaatcgatcaggaaagggattcaaagctcactttagtattaacaaaatgtggcagtcaaattttggctaatgtctctttacttgttgtaaaaggaaaatttagtaacataaacaataatactaatccaactgataaaaaaatcacagtaaagctactttttaatgaaaagggagtattaatggacagttcgacacttaagaaagaatattggaactacagaaatgataattctactgtatctcaggcctatgataatgcagttccttttatgccaaacataaaagcttatcctaaacctaccacagacacttcggctaaaccagaagataaaaaaagtgctgctaaaagatacattgtgagcaatgtctatattggaggcttgccagataaaactgttgttataactattaagtttaatgcagaaactgaatgtgcttattcgattacctttgaattcacatgggcaaaaacctttgaagatgtgcagtttgattcctcctcttttaccttttcctatattgcccaagaaaatgaggacgaagacaaataaaatgttttaaaatgaattcatgtatctttattgatttttacaccagcacgggtagtcagtctcccaccaccagcccatttcacagtgtaaacgattctctcagcacgggtggccttaaatagggaaatgttctgattagtgcgggaactggacttggggtctataatccacacagtttcctggcgagccaaacgggggtcggtgattgagatgaagccgtcctctgaaaagtcatccaagcgggcctcacagtccaaggtcacagtctggtgaaacgagaagaacgcacagattcatactcggaaaacaggatgggtctgtgcctctccatcagcgccctcaacagtctctgccgccggggctcggtgcggctgctgcagatgggatcgggatcacaagtctctctgactatgatccccacagccttcagcatcagtctcctggtgcgtcgggcacagcaccgcatcctgatctcgctcatgttctcacagtaagtgcagcacataatcaccatgttattcagcagcccataattcagggtgctccagccaaaactcatgttggggatgatggaacccacgtgaccatcgtaccagatgcggcagtatatcagatgcctgcccctcatgaacacactgcccatatacatgatctctttgggcatgtctctgttcacaatctgacggtaccagggaaagcgctggttgaacatgcacccgtaaatgactctcctgaaccacacggccagcagggtgcctcccgcccgacactgcagggagcccggggatgaacagtggcaatgcaggatccagcgctcgtacccgctcaccatctgagctctcaccaagtccagggtagcggggcacaggcacactgacatacatctttttaaaatttttatttcctctggagtcaagatcatatcccaggggactggaaactcttggagcagggtaaagccagcagcacatggtaatccacggacagaacttacattatgataatctgcatgatcacaatcaggcaacaggggatgttgttcagtcagtgaagccctggtttcctcatcagatcgtggtaaacgggccctgcgatatggatgatggcggagcgagctggattgaatctcggtttgcattgtagtggattctcttgcgtaccttgtcgtacttctgccagcagaaatgggcccttgaacagcagatacccctcctgcggccgtcctttcgctgctgccgctcagtcatccaactgaagtacatccattctcgaagattctggagaagttcctctgcatctgatgaaacaaaaaacccgtccatgcgaattcccctcatcacatcagccaggactctgtaggccatccccatccagttaatgctgccttgtctatcattcagagggggcggtggcaggattggaagaaccatttttattccaaacggtctcgaaggacgataaagtgcaagtcacgcaggtgacagcgttcccctccgctgtgctggtggaaacagacagccaggtcaaaacccactctattttcaaggtgctcgaccgtggcttcgagcagtggctctacgcgtacatccagcataagaatcacattaaaggctggccctccatcgatttcatcaatcatcaggttacattcctgcaccatccccaggtaattctcatttttccagccttggattatctctacaaattgttggtgtaagtccactccgcacatgtggaaaagctcccacagtgccccctccactttcataatcaggcagaccttcataatagaaacagatcctgctgctccaccacctgcagcgtgttcaaaacaacaagattcaataaggttctgccctccgccctgagctcgcgcctcaatgtcagctgcaaaaaatcacttaagtcctgggccactacagctgacaattcagagccagggctaagcgtgggactggcaagcgtaagggaaaactttaatgctccaaagctagcacccaaaaactgcatgctggaataagctctctttgtgtctccggtgatgccttccaaaatgtgagtgataaagcgtggtagtttttctttaatcatttgcgtaatagaaaagtcctgtaaataagtcactaggaccccagggaccacaatgtggtagcttacaccgcgtcgctgaagcatggttagtagagatgagagtctgaaaaacagaaagcatgcactaaactaaggtggctattttcactgaaggaaaaatcactctctccaacaacagggtacccactgggtggcccttgcggacatacaaaaatcggtccgtgtgattaaaaagcagcacagtaagttcctgtcttcttccggcaaaaatcacatcggactgggttagtatgtccctggcatggtagtcattcaaggccataaatctgccctgatatccagtaggaaccagcacactcacttttaggtgaagcaataccaccccatgcggaggaatgtggaaagattcagggcaaaaaaaattatatctattgctagtcccttcctggacgggagcaatccctccaggactatctatgaaagcatacagagattcagccatagctcagcccgcttaccagtagacagagagcacagcagtacaagcgccaacagcagcgactgactacccactgacccagctccctatttaaaggcgccttacactgacgtaatgaccaaaggtctaaaaaccccgccaaaaaaaaacacacacgccctgggtgttttttgcgaaaacacttccgcgttctcacttcctcgtattgatttcgtgacttaacttccgggttcccacgttacgtcacttctgcccttacatgtaactcagtcgtagggcgccatcttgcccacgtccaaaatggcttccatgtccagccacgcctccgcggcgaccgttagccgtgcgtcgtgacgtcatttgcatcatcttctctcgtccaatcagcgctggccccgccctaaattcaaaagctcatttgcatgttaacttttgtttactttgtggggtatattattgatgatc

SEQ ID NO: 6 - DNA, 19 bp, Artificial Sequence
  oligonucleotide
actcgagatgtatagatct

SEQ ID NO: 7 - DNA, 20 bp, Artificial Sequence
  oligonucleotide
ctagatctatacatctcgag

SEQ ID NO: 8 - DNA, 26 bp, Artificial Sequence
  PCR primer
gcacagatctttgcttcaggaatatg

SEQ ID NO: 9 - DNA, 29 bp, Artificial Sequence
  PCR primer
gagaactcgaggcctacatgggggtagag

SEQ ID NO: 10 - DNA, 26 bp, Artificial Sequence
  PCR primer
cgagacggccgacgcagatctgtttg

SEQ ID NO: 11 - DNA, 25 bp, Artificial Sequence
  PCR primer
gaagtcccgggctacatgggggtag

SEQ ID NO: 12 - DNA, 34775 bp, adenovirus serotype 34
catcatcaataatataccttatagatggaatggtgccaatatgtaaatgaggtgattttaaaaattgtggggtgtgtggtgattggctgtggggttaacggctaaacggggcggcgcggccgtgggaaaatgacgttttgtgggggtggagtttttttgcaagttgtcgcgggaaatgtgacgcataaaaaggctttttttctcacggaactactgacttttcccacggtatttaacaggaaatgaggtagttttgaccggatgcaagtgaaaattgctgatttgcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctacggtatttatacctcagggtttgtgtcaagaggccactcttgagtgccagcgagaagagttttctcctctgcgccggcagtttaataataaaaaaatgagagatttgcgatttctgcctcaggaaataatttctgctgagactggaaatgaaatactggagcttgtggtgcacgccctgatgggagacgatccggagccacctgtgcagctttttgagcctcctacgcttcaggaactgtatgatttagaggtagagggatcggaggattctaatgaggaagctgtgaatggcttttttaccgattctatgcttttagctgctaatgaaggattagaattagatccgcctttggacactttcgatactccaggggtgattgtggaaagcggtacaggtgtaagaaaattacctgatttgggttccgtggactgtgatttgcactgctatgaagacgggtttcctccgagtgatgaggaggaccatgaaaaggagcagtctatgcagactgcagcgggtgagggagtgaaggctgccagtgttggttttcagttggattgcccggagcttcctggacatggctgtaagtcttgtgaatttcacaggaaaaatactggagtaaaggaactgttatgttcgctttgttatatgagagcgcactgccactttatttacagtaagtgtgtttaagttaaaatttaaaggaatatgctgtttttcacatgtatattgagtgggagttttgtgcttcttattataggtcctgtgtctgatgctgatgagtcaccatctcctgattctactacctcacctcctgagattcaagcacctgttcctgtggacgtgcgcaagcccattcctgtgaagcttaagcctgggaaacgtccagcagtggaaaaacttgaggacttgttacagggtggggacggacctttggacttgagtacacggaaacggccaagacaataagtgttccatatccgtgtttacttaaggtgacgtcaatatttgtgtgagagtgcaatgtaataaaaatatgttaactgttcactggtttttattgctttttgggcggggactcaggtatataagtagaagcagacctgtatggttagctcataggagctggctttcatccatggaggtttgggccattttggaagaccttagaaagactaggcaactgttagaggacgcttcggacggagtctccggtttttggagattctggttcgctagtgaattagctagggtagtttttaggataaaacaggactataaagaagaatttgaaaagttgttggtagattgcccaggactttttgaagctcttaatttgggccatcaagttcactttaaagaaaaagttttatcagttttagacttttcaaccccaggtagaactgccgctgctgtggcttttcttacttttatattagataaatggatcccgcagactcatttcagcaggggatacgttttggatttcgtagccacagcattgtggagaacatggaaggttcgcaagatgaggacaatcttaggttactggccagtgcagcctttgggtgtagcgggaatcctgaggcatccaccggtcatgccagcggttctggaggaggaacagcaagaggacaacccgagagccggcctggaccctccagtggaggaggcggagtagctgacttgtctcctgaactgcaacgggtgcttactggatctacgtccactggacgggataggggcgttaagagggagagggcatctagtggtactgatgctagatctgagttggctttaagtttaatgagtcgcagacgtcctgaaaccatttggtggcatgaggtccagaaagagggaagggatgaagtttctgtattgcaggagaaatattcactggaacaggtgaaaacatgttggttggagcctgaggatgattgggaggtggccattaaaaattatgccaagatagctttgaggcctgataaacagtataagattactagacggattaatatccggaatgcttgttacatatctggaaatggggctgaggtggtaatagatactcaagacaaggcagttattagatgctgcatgatggatatgtggcctggagtagtcggtatggaagcagtaacttttgtaaatgttaagtttaggggagatggttataatggaatagtgtttatggccaataccaaacttatattgcatggttgtagcttttttggtttcaacaatacctgtgtagatgcctggggacaggttagtgtacggggatgtagtttctatgcgtgttggattgccacagctggcagaaccaagagtcaattgtctctgaagaaatgcatattccaaagatgtaacctgggcattctgaatgaaggcgaagcaagggtccgccactgcgcttctacagatactggatgttttattttaattaagggcaatgccagcgtaaagcataacatgatttgcggtgcttccgatgagaggccttatcaaatgctcacttgtgccggtgggcattgtaatatgctggctactgtgcatattgtttcccatcaacgcaaaaaatggcctgtttttgatcacaatgtgttgaccaagtgtaccatgcatgcaggtgggcgtagaggaatgtttatgccttaccagtgtaacatgaatcatgtgaaagtgttgttggaaccagatgccttttccagaatgagcctaacaggaatctttgacatgaacatgcaaatctggaagatcctgaggtatgatgatacgagatcgagggtgcgcgcatgcgaatgcggaggcaagcatgccaggttccagccggtgtgtgtagatgtgactgaagatctgagaccggatcatttggttattgcccgcactggagcagagttcggatccagtggagaagaaactgactaaggtgagtattgggaaaacttggggtggggttttcagatggacagattgagtaaaaatttgttttttctgtctttcagctgtcatgagtggaaacgcttcttttaaggggggagtcttcagcccttatctgacagggcgtctcccatcctgggcaggagttcgtcagaatgttatgggatctactgtggatggaagacccgtccaacccgccaattcttcaacgctgacctatgctactttaagttcttcacctttggacgcagctgcagccgccgccgccgcctctgttgccgctaacactgtgcttggaatgggttactatggaagtatcgtggctaattccacttcctctaataacccttctaccctgactcaggacaagttacttgtccttttggcccagctggaggctttgacccaacgtctgggtgaactttatcagcaggtggccgagttgcgagtacaaactgagtctgctgtcggcacggcaaagtctaaataaaaaaaaattccacaatcaatgaataaataaacgagcttgttgttgatttaaaatcaagtgtttttatttcatttttcgcgcacggtatgccctagaccaccgatctcgatcattgagaacacggtggattttttccagaatcctatagaggtgggattgaatgtttagatacatgggcattaggccatctttggggtggagatagctccattgaagggattcatgctccggggtagtgttgtaaatcacccagtcataacaaggtcgcagtgcatggtgttgcacaatatcttttagaagtaggctgattgccacagataagcccttggtgtaggtgtttacaaaccggttgagctgggaggggtgcattcggggtgaaattatgtgcattttggattggatttttaagttggcaatattgccgccaagatctcgtcttgggttcatgttatgaaggaccaccaagacggtgtatccggtacatttaggaaatttatcgtgtagcttggatggaaaagcgtggaaaaatttggagacacccttgtgtcctccgagattttccatgcactcatccatgataatagcaatggggccgtgggcagcagcgcgggcaaacacgttccgtgggtctgacacatcatagttatgttcctgagttaaatcatcataagccattttaatgaatttggggcggagagtacccgattggggtatgaatgttccttcgggccccggagcatagttcccctcacagatttgcatttcccaagctttcagttccgatggtggaatcatgtccacctggggggctatgaagaacaccgtttctggggcgggggtgattagttgggatgatagcaagtttctgagcaattgagatttgccacatccggtggggccataaatgattccgattacaggttgcaggtggtagtttagggaacggcaactgccgtcttctcgaagcaagggggccacctcgttcatcatttcccttacatgcatattttcccgcaccaaatccattaggaggcgctctcctcctagtgatagaagttcttgtagtgaggaaaagtttttcagcggttttagaccgtcagccatgggcattttggagagagtttgctgcaaaagttctagtctgttccacagttcagtgatgtgttctatggcatctcgatccagcagacctcctcgtttcgcgggtttggacggctcctggagtagggtatgagacgatgggcgtccagcgctgccagggttcggtccttccagggtctcagtgttcgagtcagggttgtttccgtcacagtgaaggggtgtgcgcctgcttgggcgcttgccagggtgcgcttcagactcattctgctggtggagaacttctgtcgcttggcgccctgtatgtcggccaagtagcagtttaccatgagttcgtagttgagcgcctcggctgcgtggcctttggcgcggagcttacctttggaagttttcttgcataccgggcagtataggcatttcagcgcatacagcttgggcgcaaggaaaatggattctggggagtatgcatctgcgccgcaggaggcgcaaacagtttcacattccaccagccaggttaaatccggttcattggggtcaaaaacaagttttccgccatattttttgatgcgtttcttacctttggtctccatgagttcgtgtcctcgttgagtgacaaacaggctgtccgtatccccgtagactgattttacaggcctcttctccagtggagtgcctcggtcttcttcgtacaggaactctgaccactctgatacaaaggcgcgcgtccaggccagcacaaaggaggctatgtgggaggggtagcgatcgttgtcaaccagggggtccaccttttccaaagtatgcaaacacatgtcaccctcttcaacatccaggaatgtgattggcttgtaggtgtatttcacgtgacctggggtccccgctgggggggtataaaagggggcggttctttgctcttcctcactgtcttccggatcgctgtccaggaacgtcagctgttggggtaggtattccctctcgaaggcgggcatgacctctgcactcaggttgtcagtttctaagaacgaggaggatttgatattgacagtgccggttgagatgcctttcatgaggttttcgtccatttggtcagaaaacacaatttttttattgtcaagtttggtggcaaatgatccatacagggcgttggataaaagtttggcaatggatcgcatggtttggttcttttccttgtccgcgcgctctttggcagcgatgttgagttggacatactcgcgtgctaggcacttccattcggggaagatagttgtcaattcatctggcacgattctcacttgccaccctcgattatgcaaggtaattaaatccacactggtggccacctcgcctcgaaggggttcgttggtccaacagagcctacctcctttcctagaacagaaagggggaagtgggtctagcataagttcatcgggagggtctgcatccatggtaaagattcccggaagtaaatccttatcaaaatagctgatgggagtggggtcatctaaggccatttgccattctcgagctgccagtgcacgctcatatgggttaaggggactgccccagggcatgggatgggtgagtgcagaggcatacatgccacagatgtcatagacgtagatgggatcctcaaagatgcctatataggttggatagcatcgcccccctctgatacttgctcgcacatagtcatatagttcatgtgatggcgctagcaaccccggacccaagttggtgcgattgggtttttctgttctgtagacaatctggcgaaagatggcgtgagaattggaagagatggtgggtctttgaaaaatgttgaaatgggcatgaggtagacctacagagtctctgacaaagtgggcataagattcttgaagcttggttaccagttcggcggtgacaagtacgtctagggcgcagtagtcaagtgtttcttgaatgatgtcataacctggttggtttttcttttcccacagttcgcggttgagaaggtattcttcgcgatccttccagtactcttctagcggaaacccgtctttgtctgcacggtaagatcctagcatgtagaactgattaactgccttgtaagggcagcagcccttctctacgggtagagagtatgcttgagcagcttttcgcagcgaagcgtgagtaagggcgaaggtgtctctgaccatgactttgagaaattggtatttgaagtccatgtcgtcacaggctccctgttcccagagttggaagtctacccgtttcttgtaggcggggttgggcaaagcgaaagtaacatcgttgaagagaatcttaccggctctgggcataaaattgcgagtgatgcggaaaggctgtggtacttccgctcgattgttgatcacctgggcagctaggacgatctcgtcgaaaccgttgatgttgtgtcctacgatgtataattctatgaaacgcggcgtgcctttgacgtgaggtagcttattgagctcatcaaaggttaggtctgtagggtcagataaggcgtagtgttcgagagcccattcgtgcaggtgaggatttgcatgtaggaatgatgaccaaagatccaccgccagtgctgtttgtaactggtcccgatactgacgaaaatgctggccaattgccattttttctggagtgacacagtagaaggttctggggtcttgttgccatcgatcccactttagtttaatggctagatcgtgggccatgttgacgagacgctcttctcctgagagtttcatgaccagcatgaaaggaactagttgtttgccaaaggaccccatccaggtgtaagtttccacatcgtaggtcaggaagagtctttctgtgcgaggatgagagccgatcgggaagaactggatttcctgccaccagttggaggattggctgttgatgtgatggaagtagaagtttctgcggcgcgccgagcattcgtgtttgtgcttgtacagacggccgcagtagtcgcagcgttgcacgggttgtatctcgtgaatgagctgtacctggcttcccttgacgagaaatttcagtgggaagccgaggcctggcgattgtatctcgtgctcttctatattcgctgtatcggcctgttcatcttctgtttcggtggtggtcatgctgacgagcccccgcgggaggcaagtccagacctcggcgcgggaggggcggagctgaaggaccagagcgcgcaggctggagctgtccagagtcctgagacgctgcggactcaggttagtaggtagggacagaagattaacttgcatgatcttttccagggcgtgcgggaggttcagatggtacttgatttccacaggttcgtttgtagagatgtcaatggcttgcagggttccgtgtcctttgggcgccactaccgtacctttgttttttcttttgatcggtggtggctctcttgcttcttgcatgctcagaagcgatgacggggacgcgcgccgggcggaagcggttgttccggacccggaggcatggctggtagtggcacgtcggcgccgcgcacgggcaggttctggtactgcgctctgagaagacttgcgtgcgccaccacgcgtcgattgacgtcttgtatctgacgtctctgggtgaaagctaccggccccgtgagcttgaacctgaaagagagttcaacagaatcaatttcggtatcgttaacggcagcttgtctcagtatttcttgtacgtcaccagagttgtcctggtaggcgatctccgccatgaactgctcgatttcttcctcctgaagatctccgcgacccgctctctcgacggtggccgcgaggtcattggagatacggcccatgagttgggagaatgcagtcatgcccgcctcgttccagacgcggctgtaaaccacggccccctcggagtctcttgcgcgcatcaccacctgagcgaggttaagctccacgtgtctggtgaagaccgcatagttgcataggcgctgaaaaaggtagttgagtgtggtggcaatgtgttcggcgacgaagaaatacatgatccatcgtctcagcggcatttcgctgacatcgcccagagcttccaagcgctccatggcctcgtagaagtccacggcaaaattaaaaaactgggagtttcgcgcggacacggtcaattcctcctcgagaagacggatgagttcggctatggtggcccgtacttcgcgttcgaaggctcccgggatctcttcttcctcttctatctcttcttccactaacatctcttcttcgtcttcaggcgggggcggagggggcacacggcgacgtcgacggcgcacgggcaaacggtcgatgaatcgttcaatgacctctccgcggcggcggcgcatggtttcagtgacggcgcggccgttctcgcgcggtcgcagagtaaaaacaccgccgcgcatctccttaaagtggtgactgggaggttctccgtttgggagggagagggcgctgattatacattttattaattggcccgtagggactgcgcgcagagatctgatcgtgtcaagatccacgggatctgaaaacctttcgacgaaagcgtctaaccagtcacagtcacaaggtaggctgagtacggcttcttgtgggcgggggtggttatgtgttcggtctgggtcttctgtttcttcttcatctcgggaaggtgagacgatgctgctggtgatgaaattaaagtaggcagttctaagacggcggatggtggcgaggagcaccaggtctttgggtccggcttgctggatacgcaggcgattggccattccccaagcattatcctgacatctagcaagatctttgtagtagtcttgcatgagccgttctacgggcacttcttcctcacccgttctgccatgcatacgtgtgagtccaaacccgcgcattggttgtaccagtgccaagtcagctacgactctttcggcgaggatggcttgctgtacttgggtgagggtggcttgaaagtcatcaaaatccacaaagcggtggtaagccccggtattaatggtgtaagcacagttggccatgactgaccagttaactgtctggtgaccagggcgcacgagctcggtgtatttaaggcgcgaataggcgcgggtgtcaaagatgtaatcgttgcaggtgcgcaccagatactggtaacctataagaaaatgcggcggtggttggcggtagagaggccatcgttctgtagctggagcgccgggggcgaggtcttccaacataaggcggtgatagccgtagatgtacctggacatccaggtgattcctgcggcggtagtagaagcccgaggaaactcgcgtacgcggttccaaatgttgcgtagcggcatgaagtagttcattgtaggcacggtttgaccagtgaggcgcgcgcagtcattgatgctctatagacacggagaaaatgaaagcgttcagcgactcgactccgtagcctggaggaacgtgaacgggttgggtcgcggtgtaccccggttcgagacttgtactcgagccggccggagccgcggctaacgtggtattggcactcccgtctcgacccagcctacaaaaatccaggatacggaatcgagtcgttttgctggttgccgaatggcagggaagtgagtcctattttttttttttgccgctcagatgcatcccgtgctgcgacagatgcgtccccaacaacagcccccctcgcagcagcagcaaccacaaaaggctgtccctgcaactactgcaactgccgctgtgagcggtgcgggacagcccgcctatgatctggacttggaagagggcgaaggactggcacgtctaggtgcgccttcgcccgagcggcatccgcgagttcaactgaaaaaagattctcgcgaggcgtatgtgccccaacagaacctatttagagacagaagcggcgaggagccggaggagatgcgagcttcccgctttaacgcgggtcgtgagctgcgtcacggtttggacagaagacgagtgttgcgggacgaggatttcgaagttgatgaagtgacagggatcagtcctgccagggcacacgtggctgcagccaaccttgtatcggcttacgaacagacagtaaaggaagagcgtaatttccaaaagtcttttaataatcatgtgcgaaccctcattgcccgcgaagaagtcacccttggtttgatgcatttgtgggatttgatggaagctatcattcagaaccctactagcaaacctctgaccgcacagctgtttctggtggtgcaacacagcagagacaatgaggctttcagagaggcgctgctcaacatcaccgaacccgaggggagatggttgtatgatcttatcaacattctacagagtatcatagtgcaggagcggagcctgggcctggccgagaaggtggctgccatcaattactcggttttgagcttgggaaagtattacgctcgcaagatctacaagactccatacgttcccatagacaaggaggtgaagatagatgggttctacatgcgcatgacgctgaaggtgttgaccctgagcgatgatcttggggtgtaccgcaatgacagaatgcatcgcgcggtgagcgccagcaggaggcgcgagttaagcgacagggaactgatgcacagtttgcaaagagctctaactggagctggaaccgagggtgagaattactttgatatgggagctgacttgcagtggcagcctagtcgcagggctctgaacgccgcgacggcaggatgtgagcttccttacatagaagaggcggatgaaggcgaggaggaagagggcgagtacttggaagactgatggcacaacccgtgttttttgctagatggaacagcaagcaccggatcccgcaatgcgggcggcgctgcagagccagccgtccggcattaactcctcggacgattggacccaggccatgcaacgtatcatggcgttgacgactcgcaaccccgaagcctttagacagcaaccccaggccaaccgtctatcggccatcatggaagctgtagtgccttcccgctctaatcccactcatgagaaggtcctggccatcgtgaacgcgttggtggagaacaaagctattcgtccagatgaggccggactggtatacaacgctctcttagaacgcgtggctcgctacaacagtagcaatgtgcaaaccaatttggaccgtatgataacagatgtacgcgaagccgtgtctcagcgcgaaaggttccagcgcgatgccaacctgggttcgctggtggcgttaaatgctttcttgagtactcagcctgctaatgtgccgcgtggtcaacaggattatactaactttttaagtgctttgagactgatggtatcagaagtacctcagagcgaagtatatcagtccggtcctgattacttctttcagactagcagacagggcttgcagacggtaaatctgagccaagcttttaaaaaccttaaaggtttgtggggagtgcatgccccggtaggagaaagagcaaccgtgtctagcttgttaactccgaactcccgcctattattactgttggtagctcctttcaccgacagcggtagcatcgaccgtaattcctatttgggttacctactaaacctgtatcgcgaagccatagggcaaagtcaggtggacgagcagacctatcaagaaattacccaagtcagtcgcgctttgggacaggaagacactggcagtttggaagccactctgaacttcttgcttaccaatcggtctcaaaagatccctcctcaatatgctcttactgcggaggaggagaggatccttagatatgtgcagcagagcgtgggattgtttctgatgcaagagggggcaactccgactgcagcactggacatgacagcgcgaaatatggagcccagcatgtatgccagtaaccgacctttcattaacaaactgctggactacttgcacagagctgccgctatgaactctgattatttcaccaatgccatcttaaacccgcactggctgcccccacctggtttctacacgggcgaatatgacatgcccgaccctaatgacggatttctgtgggacgacgtggacagcgatgttttttcacctctttctgatcatcgcacgtggaaaaaggaaggcggcgatagaatgcattcttctgcatcgctgtccggggtcattggtgctaccgcggctgagcccgagtctgcaagtccttttcctagtctacccttttctctacacagtgtacgtagcagcgaagtgggtagaataagtcgcccgagtttaatgggcgaagaggagtacctaaacgattccttgctcagaccggcaagagaaaaaaatttcccaaacaatggaatagaaagtttggtggataaaatgagtagatggaagacttatgctcaggatcacagagacgagcctgggatcatggggactacaagtagagcgagccgtagacgccagcgccatgacagacagaggggtcttgtgtgggacgatgaggattcggccgatgatagcagcgtattggacttgggtgggagaggaaggggcaacccgtttgctcatttgcgccctcgcttgggtggtatgttgtaaaaaaaaataaaaaagaaaaaactcaccaaggccatggcgacgagcgtacgttcgttcttctttattatctgtgtctagtataatgaggcgagtcgtgctaggcggagcggtggtgtatccggagggtcctcctccttcgtacgagagcgtgatgcagcagcagcaggcgacggcggtgatgcaatccccactggaggctccctttgtgcctccgcgatacctggcacctacggagggcagaaacagcattcgttactcggaactggcacctcagtacgataccaccaggttgtatctggtggacaacaagtcggcggacattgcttctctgaactatcagaatgaccacagcaacttcttgaccacggtggtgcaaaacaatgactttacccctacggaagccagcacccagaccattaactttgatgaacgatcgcggtggggcggtcagctaaaaaccatcatgcatactaacatgcccaacgtgaacgagtatatgtttagtaacaagttcaaagcgcgtgtgatggtgtccagaaaacctcctgagggtgttagagtagacgataattatgatcataagcaagatattctaaaatacgagtggttcgagtttactttgccagaaggcaacttttcggtcactatgactatcgacttgatgaacaatgccatcatagacaattacttgaaagtgggcagacagaatggagtgttggaaagtgacattggtgttaagttcgacactaggaacttcaagttgggatgggatccagaaactaagttgatcatgcctggggtttacacctatgaggccttccatcctgacatcgtattgctgcctggctgcggagtggactttaccgaaagccgtctgagcaaccttcttggcattagaaagaaacacccattccaagagggttttaagatcttgtatgaggatttagaaggaggaaatattccagcccttttggatgtagatgcttatgagaacagcaagaaagatcaaaaagccaaaatagaagctgctgcagaagctaaagcaaacatagttgccaacgatccggtaagggtggctaacgctagtgaaatcaggggagacagttttgccgcaacatccgttccgactaaagaatcattattggatgatgtgtctcaaaacatagagttaaaactcactattaagcctgtggaaaaagatggcaaaaacagaagttacaatgtgttggaagataaaatcaacacggcctatcgcagttggtacctttcgtacaattatggcgaccccgaaaaaggagtgcgttcctggacattgctcaccacctcagatgtcacctgcggagcggagcaggtctactggtcgcttccagacatgatgcaggatcctgtcactttccgctccactagacaagtcagtaactaccctgtggtgggtgcagagcttatgcccgtcttttcaaagagcttctacaacgaacaagctgtgtactcccagcagctccgccagtccacctcgcttacgcacgtcttcaaccgctttcctgagaaccagattttaatccgtccgccggcgcccacaattaccaccgtcagtgaaaacgttcctgctctcacagatcacgggaccctgccgttgcgcagcagtatccggggagtccaacgtgtgaccgttactgacgccagacgccgcacctgtccctacgtgtacaaggcactgggcatagtcgcaccgcgcgtcctttcaagccgcactttctaaaaaaaaaaaaaaaatgtccgttcttatctcgcccagtaataacaccggttggggtctgcgcgctcccagcaagatgtacggaggcgcacgcaaacgttctacccaacatcccgtgcgtgttcgcgggcattttcgcgctccatggggtgccctcaagggccgcactcgcgttcgaaccaccgtcgatgatgtaatcgatcaggtggttgccgacgcccgtaattatactcctactgcgcctacatctactgtggacgcagttattgacagtgtagtggctgacgctcgcaactatgctcgacgtaagagccggcgaaggcgcattgccagacgtcaccgagctaccactgccatgcgagcagcaagagctctgctacgaagagctagacgcgtggggcgaagagccatgcttagggcggccagacgtgcagcttcgggcgccagcgccggcaggtcccgcaggcaagcagccgctgtcgcagcggcgactattgccgacatggcccaatcgcgaagaggcaatgtatactgggtgcgtgacgctgccaccggtcaacgtgtacccgtgcgcacccgtccccctcgcacttagaagatactgagcagtctccgatgttgtgtcccagcggcgaggatgtccaagcgcaaatacaaggaagaaatgctgcaggttatcgcacctgaagtctacggccaaccgttgaaggatgaaaaaaaaccccgcaaaatcaagcgggtaaaaaaggacaaaaaagaagaggaagatggcgatgatgggctggcggagtttgtgcgcgagtttgccccacggcgacgcgtgcaatggcgtgggcgcaaagttcgacatgtgttgagacctggaacttcggtggtctttacacccggcgagcgttcaagcgctacttttaagcgttcctatgatgaggtgtacggggatgatgatattcttgagcaggcagctgaccgattaggcgagtttgcttatggcaagcgtagtagaataaatcccaaggatgaaacagtgtccatacccttggatcatggaaatcccacccctagtcttaaaccggtcactttgcagcaagtgttacccgtaactccgcgaacaggtgttaaacgcgaaggtgaagatttgtatcccactatgcaactgatggtgcccaaacgccagaagttggaggacgttttggagaaagtaaaagtggatccagatattcaacctgaggttaaagtgagacccattaagcaggtagcgcctggtctgggagtacaaactgtagacattaaaattcccactgaaagtatggaagtgcaaactgaacccgcaaagcctactgccacctccactgaagtgcaaacggacccatggatgcccatgcctattacaactgacgccgtcggtcccactcgaagatcccgacgaaagtacggtccagcaagtctgttgatgcccaactatgtcgtacacccatctattattcctactcctggttaccgaggcactcgctactatcgcagccgaaacagtacttcccgccgtcgccgcaagacacctgcaaatcgcagtcgtcgccgtagacgcacaagcaaaccgattcccggcgccctggtgcggcaagtgtaccgcaatggtagtgcggaacctttgacactgccgcgtgcgcgttaccatcctagtatcatcacttaatcaatgttgccgctgcctccttgcagatatggccctcacttgtcgccttcgcgttcccatcactggttaccgaggaagaaactcgcgccgtagaagagggatgttggggcgcggaatgcgacgctacaggcgacggcgtgctatccgcaagcaattgcggggtggttttttgccagccttaattccaattatcgctgctgcgattggcgcaataccaggcatagcttccgtggcggttcaggcctcgcaacgacattgacattggaaaaaaaaaaaacgtataaataaaaaatacaatggactctgacactcctggtactgtgactatgttttcttagagatggaagacatcaatttttcatccttggctccgcgacacggcacgaagccgtacatgggcacctggagcgacatcggcacgagccaactgaacgggggcgccttcaattggagcagtatctggagcgggcttaaaaattttggctcaaccataaaaacatacgggaacaaagcttggaacagcagtacaggacaggcgcttagaaataaacttaaagaccagaacttccaacaaaaagtagtcgatgggatagcttccggtatcaatggagtggtagatttggctaaccaggctgtgcagaaaaagataaacagtcgtttggacccgccgccagcaaccccaggtgaaatgcaagtggaggaagaaattcctccgccagaaaaacgaggcgacaagcgtccgcgtcccgatttggaagagacgctggtgacgcgcgtagatgaaccgccttcttatgaggaagcaacgaagcttggaatgcccaccactagaccgatagcccctatggccaccggggtgatgaaaccttctcagttgcatcgacccgtcaccttggatttgccccctcctcctgctgctactgctgtacccgcttctaagcctgtcgctgccccgaaaccagtcgccgtagccaggtcacgtcccgggggcgctcctcgtccaaatgcacactggcaaaatactctgaacagcatcgtgggtctaggcgtgcaaagtgtaaaacgccgtcgctgcttttaattaaatatggagtagcgcttaacttgcctatctgtgtatatgtgtcattacacgccgtcacagcatcagaggaaaaaaggaagaggtcgtgcgtcgacgctgagttactttcaagatggccaccccatcgatgctgccccaatgggcatacatgcacatcgccggacaggatgcttcggagtacctgagtccgggtctggtgcagttcgcccgcgccacagacacctacttcaatctgggaaataagtttagaaatcctaccgtagcgccgacccacgatgtgaccaccgatcgtagccagcggctcatgttgcgcttcgtgcccgttgaccgggaggacaatacatactcttacaaagtgcggtacaccctggccgtgggcgacaacagagtgctggatatggccagcacgttctttgacattaggggcgtgttggacagaggtcccagttttaaaccctattctggtacggcttacaactccctggctcctaaaggcgctccaaatgcatctcagtggttggataagggagttacaagcactggcctagtggacgacggcaatactgatgatggggaagaagccaaaaaagcaacatacacttttggtaatgctccagtaaaagccgaggctgaaatcacaaaagacggattgccggtgggcttggaagtttcaactgaaggtcctaaaccaatctatgctgataagctttatcagccagaacctcaagtgggagacgaaacttggactgacctagacggaaaaaccgaagagtatggagggagggttcttaaacctgaaactaaaatgaaaccctgctacggatcttttgctaaacctactaatattaaaggaggtcaggcaaaggtaaaaccaaaagaagacgatggcactaacaacatcgagtatgacattgacatgaacttctttgacttaagatcacaaagatcagaactcaaacctaaaattgtaatgtatgcagaaaatgtggacctggaatgtccagatactcatgttgtgtacaaacctggagtttcagatgctagttctgagaccaatcttggacaacagtctatgcccaacagacccaactacattggcttcagagataacttcatcggacttatgtactataacagtactggcaacatgggggtactggctggccaagcgtctcagttgaatgcagtggttgacttgcaggacagaaacacagaactgtcttaccaactcttgcttgactctctgggcgacagaaccagatactttagcatgtggaatcaggctgtggacagttatgatcctgatgtacgtgttattgaaaatcatggtgtggaagatgaacttcccaactattgttttccgttggatggtgtcggtccgcgaacagatagttacaaggagattaagccaaatggagaccaatctacttggacaaatgtagacccaactggcagcagtgaacttgctaagggaaatccatttgccatggaaattaaccttcaagccaatctatggcgaagtttcctttattccaatgtggctctatatctcccagactcgtacaaatacaccccgtccaatgtcactcttccagaaaacaaaaacacctacgactacatgaacgggcgggtggtgccgccatctctagtagacacctatgtgaacattggtgccaggtggtctctggatgccatggacaatgtcaacccattcaaccaccaccgtaacgctggcttgcgttaccgatccatgcttctgggtaacggacgttatgtgcctttccacatacaagtgcctcaaaaattcttcgctgttaaaaacctgctgcttctcccaggctcctacacttatgagtggaactttaggaaggatgtaaacatggttctacagagttccctcggtaacgacctacgggtagatggcgccagcatcagttttacgagcatcaacctctatgctacttttttccccatggctcacaacaccgcttccacccttgaagccatgctgcggaatgacaccaatgatcagtcattcaacgactacctatctgcagctaacatgctctaccccattcctgccaatgcaaccaatattcccatttccattccttctcgcaactgggcggctttcagaggctggtcatttaccagactgaaaaccaaagaaactccctctttggggtctggatttgacccctacttcgtctattctggttctattccctacctggatggtaccttctacctgaaccacacttttaagaaggtttccatcatgtttgactcttcagtgagctggcctggaaatgacaggttactatctcctaacgaatttgaaataaagcgcactgtggatggcgaaggctacaacgtagcccaatgcaacatgaccaaagactggttcttggtacagatgctcgccaactacaacatcggctatcagggcttctacattccagaaggatacaaagatcgcatgtattcatttttcagaaacttccagcccatgagcaggcaggtggttgatgaggtcaattacaaagacttcaaggccgtcgccataccctaccaacacaacaactctggctttgtgggttacatggctccgaccatgcgtcaaggtcaaccctatcccgctaactatccctatccactcattggaacaactgccgtaaatagtgttacgcagaaaaagttcttgtgtgacagaaccatgtggcgcataccgttctcaagcaacttcatgtctatgggagcccttacagacttgggacagaacatgctctatgccaactcagctcatgctctggacatgacctttgaggtggatcccatggatgagcccaccctgctttatcttctcttcgaagttttcgacgtggtcagagtgcatcagccacaccgcggcatcatcgaggcagtctacctgcgtacaccgttctcggccggtaacgctaccacgtaagaagcttcttgcttcttgcaaacagcagctgcaaccatggcctgcggatcccaaaacggctccagcgagcaagagctcagagccattgtccaagacctgggttgcggaccatattttttgggaacctttgataagcgcttcccggggttcatggcccccgataagctcgcctgtgccattgtaaatacggccggacgtgagacggggggagagcactggttggctttcggttggaacccacgttctaacacctgctacctttttgatccttttggattctcggatgatcgtctcaaacagatttaccagtttgaatatgagggtctcctgcgccgcagcgctcttgctaccaaggaccggtgtattacgctggaaaaatctacccagaccgtgcagggcccccgttctgccgcctgcggacttttctgctgcatgttccttcatgcctttgtgcactggcctgaccgtcccatggacggaaaccccaccatgaaattgctaactggagtgccaaacaacatgcttcattctcctaaagtccagcccaccctgtgtgacaatcaaaaagcactctaccattttctcaatacccattcgccttattttcgctctcatcgtacacacatcgaaagggccactgcgttcgaccgtatggatgtgcaataatgattcatgtaaacaacgtgttcaataaacagcactttattttttacatgtatcgaggctctggattacttatttatttacaagtcgaatgggttctgacgagaatcagaatgacccgcaggcagtgatacgttgcggaactgatacttgggttgccacttgaattcgggaatcaccaacttgggaaccggtatatcgggcaggatgtcactccacagctttctggtcagctgcaaagctcccagcaggtcaggagccgaaatcttgaaatcacaattaggaccagtgctctgagcgcgagagttgcggtacaccggattgcagcactgaaacaccatcagcgacggatgtcttacgcttgccagcacggtgggatctgcaatcatgcccacatccagatcttcagcattggcaatgctgaacggggtcatcttgcaggtctgcctacccatggcgggcacccaattaggcttgtggttacaatcgcagtgcagggggatcagtatcatcttggcctgatcctgtctgattcctggatacacggctctcatgaaagcatcatattgcttgaaagcctgctgggctttactaccctcggtataaaacatcccgcaggacctgctcgaaaactggttagctgcgcagccggcatcattcacacagcagcgggcgtcattgttggctatttgcaccacacttctgccccagcggttttgggtgattttggttcgctcgggattctccttcaaggctcgttgtccgttctcgctggccacatccatctcgataatctgctccttctgaatcataatattgccatgcaagcacttcagcttgccctcataatcattgcagccatgaggccacaacgcacagcctgtacattcccaattatggtgggcgatctgagaaaaagaatgtatcattccctgcagaaatcttcccatcatcgtgctcagtgtcttgtgactagtgaaagttaactggatgcctcggtgctcctcgttcacgtactggtgacagatgcgcttgtattgttcgtgctgctcaggcattagtttaaaagaggttctaagttcgttatccagcctgtacttctccatcagcagacacatcacttccatgcctttctcccaagcagacaccaggggcaagctaatcggattcttaacagtgcaggcagcagctcctttagccagagggtcatctttggcgatcttctcaatgcttcttttgccatccttctcaacgatgcgcacgggcgggtagctgaaacccactgctacaagttgcgcctcttctctttcttcttcgctgtcttgactgatgtcttgcatggggacatgtttggtcttccttggcttctttttcgggggtatcggaggaggaggactgtcgctccgttccggagacagggaggattgtgacgtttcgctcaccattaccaactgactgtcggtagaagaacctgaccccacacggcgacaggtgtttctcttcgggggcagaggtggaggcgattgcgaagggctgcggtccgacctggaaggcggatgactggcagaaccccttccgcgttcgggggtgtgctccctgtggcggtcgcttaactgatttccttcgcggctggccattgtgttctcctaggcagagaaacaacagacatggaaactcagccattgctgtcaacatcgccacgagtgccatcacatctcgtcctcagcgacgaggaaaaggagcagagcttaagcattccaccgcccagtcctgccaccacctctaccctagaagataaggaggtcgacgcatctcatgacatgcagaataaaaaagcgaaagagtctgagccagacatcgaacaagacccgggctatgtgacaccggtggaacacgaggaagagttgaaacgctttctagagagagaggatgaaaactgcccaaaacagcaagcggataactatcaccaagatgctggaaatagggatcagaacaccgactacctcatagggcttgacggggaagacgcgctccttaaacatctagcaagacagtcactcatagtcaaggatgcattattggacagaactgaagtgcccatcagtgtcgaagagctcagccgcgcctacgagcttaacctattttcacctcgtactccccccaaacgtcagccaaacggcacctgcgagccaaatcctcgcttaaacttttatccagcttttgctgtgccagaagtactggctacctatcacatcttttttaaaaatcaaaaaattccagtctcctgccgcgctaatcgcacccgcgccgatgccctactcaatctgggacctggttcacgcttacctgatatagcttccttggaagaggttccaaagatcttcgagggtctgggcaataatgagactcgggccgcaaatgctctgcaaaagggagaaaatggcatggatgagcatcacagcgttctggtggaattggaaggcgataatgccagactcgcagtactcaagcgaagcgtcgaggtcacacactttgcataccccgctgtcaacctgccccctaaagtcatgacggccgtcatggaccagttactcattaagcgcgcaagtcccctttcagaagacatgcatgacccagatgcctgtgatgagggtaaaccagtggtcagtgatgagcagctaacccgatggctgggcaccgactctccccgggatttggaagagcgtcgcaagcttatgatggccgtggtgctggttaccgtagaactagagtgtcttcggcgtttctttaccgattcagaaaccttgcgcaaactcgaagagaatctgcactacacttttagacacggctttgtgcggcaggcatgcaagatatctaacgtggaactcaccaacctggtttcctacatgggtattctgcatgagaatcgcctaggacaaagcgtgctgcacagcacccttaagggggaagcccgccgtgattacatccgcgattgtgtttatctctacctgtgccacacgtggcaaaccggcatgggtgtatggcagcaatgtttagaagaacagaacctgaaagagctaaacaagctcttacagaaatctcttaaggttctgtggacagggttcgacgagcgcaccgtcgcttccgacctggcagacctcatcttcccagagcgtctcagggttactttgcgaaacggactgcctgactttatgagccagagcatgcttaacaattttcgctctttcatcctggaacgctccggtatcctgcccgccacctgctgcgcactgccctccgactttgtgcctctcacctaccgcgaatgccccccgccgctatggagtcactgctacctgttccgtctggccaactacctctcctaccactcggatgtgatcgaggatgtgagcggagacggcttgctggagtgtcactgccgctgcaatctgtgcacgccccaccggtccctagcttgcaacccccagttgatgagcgaaacccagataataggcacctttgaattgcaaggccccagcagccaaggcgatgggtcttctcctgggcaaagtttaaaactgaccccgggactgtggacctccgcctacttgcgcaagtttgccccggaagattaccacccctatgaaatcaagttctatgaggaccaatcacagcctccgaaagccgaactttcggcctgcgtcatcacccagggggcaattctggcccaattgcaagccatccaaaaatcccgccaagaatttctactgaaaaagggtaagggggtctaccttgacccccagaccggcgaggaactcaacacaaggttccctcaggatgtcccaacgacgagaaagcaagaagttgaaggtgcagccgccgcccccagaagatatggaggaagattgggacagtcaggcagaggaagcggaggaggaggacagtctggaggacagtctggaggaagacagtttggaggaggaaaacgaggaggcagaggaggtggaagaagtaaccgccgacaaacagttatcctcggctgcggagacaagcaacagcgctaccatctccgctccgagtcgaggaacccggcggcgtcccagcagtagatgggacgagaccggacgcttcccgaacccaaccagcgcttccaagaccggtaagaaggatcggcagggatacaagtcctggcgggggcataagaatgccatcatctcctgcttgcatgagtgcgggggcaacatatccttcacgcggcgctacttgctattccaccatggggtgaactttccgcgcaatgttttgcattactaccgtcacctccacagcccctactatagccagcaaatcccggcagtctcgacagataaagacagcggcggcgacctccaacagaaaaccagcagcggcagttagaaaatacacaacaagtgcagcaacaggaggattaaagattacagccaacgagccagcgcaaacccgagagttaagaaatcggatctttccaaccctgtatgccatcttccagcagagtcggggccaagagcaggaactgaaaataaaaaaccgatctctgcgttcgctcaccagaagttgtttgtatcacaagagcgaagatcaacttcagcgcactctcgaggacgccgaggctctcttcaacaagtactgcgcgctgactcttaaagagtaggcagcgaccgcgcttattcaaaaaaggcgggaattacatcatcctcgacatgagtaaagaaattcccacgccttacatgtggagttatcagccccaaatgggattggcggcaggcgcctcccaggactactccacccgcatgaattggctcagcgccgggccttctatgatttctcgagttaatgatatacgcgcctaccgaaaccaaatacttttggaacagtcagctcttaccaccacgccccgccaacaccttaatcccagaaattggcccgccgccctagtgtaccaggaaagtcccgctcccaccactgtattacttcctcgagacgcccaggccgaagtccaaatgactaatgcaggtgcgcagttagcgggcggctccaccctatgtcgtcacaggcctcggcataatataaaacgcctgatgatcagaggccgaggtatccagctcaacgacgagtcggtgagctctccgcttggtctacgaccagacggaatctttcagattgccggctgcgggagatcttccttcacccctcgtcaggctgttctgactttggaaagttcgtcttcgcaaccccgctcgggcggaatcgggaccgttcaatttgtggaggagtttactccctctgtctacttcaaccccttctccggatctcctgggcactacccggacgagttcataccgaacttcgacgcgattagcgagtcagtggacggctacgattgatgtctggtgacgcggctgagctatctcggctgcgacatctagaccactgccgccgctttcgctgctttgcccgggaactcattgagttcatctacttcgaactccccaaggatcaccctcaaggtccggcccacggagtgcggattactatcgaaggcaaaatacactctcgcctgcaacgaattttctcccagcggcccgtgctgatcgagcgagaccagggaaacaccacggtttccatctactgcatttgtaatcaccccggattgcatgaaagcctttgctgtcttatgtgtactgagtttaataaaaactgaattaagactctcctacggactgccgcttcttcaacccggattttacaaccagaagaacgaaacttttcctgtcgtccaggactctgttaacttcacctttcctactcacaaactagaagctcaacgactacaccgcttttccagaagcattttccctactaatactactttcaaaaccggaggtgagctccaaggtcttcctacagaaaacccttgggtggaagcgggccttgtagtgctaggaattcttgcgggtgggcttgtgattattctttgctacctatacacaccttgcttcactttcctagtggtgttgtggtattggtttaaaaaatggggcccatactagtcttgcttgttttactttcgcttttggaaccgggttctgccaattacgatccatgtctagacttcgacccagaaaactgcacacttacttttgcacccgacacaagccgcatctgtggagttcttattaagtgcggatgggaatgcaggtccgttgaaattacacacaataacaaaacctggaacaataccttatccaccacatgggagccaggagttcccgagtggtacactgtctctgtccgaggtcctgacggttccatccgcattagtaacaacactttcattttttctgaaatgtgcgatctggccatgttcatgagcaaacagtattctctatggcctcctagcaaggacaacatcgtaacgttctccattgcttattgcttgtgcgcttgccttcttactgctttactgtgcgtatgcatacacctgcttgtaaccactcgcatcaaaaacgccaataacaaagaaaaaatgccttaacctctttctgtttacagacatggcttctcttacatctctcatatttgtcagcattgtcactgccgctcacggacaaacagtcgtctctatccctctaggacataattacactctcataggacccccaatcacttcagaggtcatctggaccaaactgggaagcgttgattactttgatataatctgcaacaaaacaaaaccaataatagtaacttgcaacatacaaaatcttacattgattaatgttagcaaagtttacagcggttactattatggttatgacagatacagtagtcaatatagaaattacttggttcgtgttacccagttaaaaaccacgaaaatgccaaatatggcaaagattcgatccgatgacaattctctagaaacttttacatctcccaccacacccgacgaaaaaaacatcccagattcaatgattgcaattgttgcagcggtggcagtggtgatggcactaataataatatgcatgcttttatatgcttgtcgctacaaaaagtttcatcctaaaaaacaagatctcctactaaggcttaacatttaatttctttttatacagccatggtttccactaccacattccttatgcttactagtcttgcaactctgacttctgctcgctcacacctcactgtaactataggctcaaactgcacactaaaaggacctcaaggtggtcatgtcttttggtggagaatatatgacaatggatggtttacaaaaccatgtgaccaacctggtagatttttctgcaacggcagagacctaaccattatcaacgtgacagcaaatgacaaaggcttctattatggaaccgactataaaagtagtttagattataacattattgtactgccatctaccactccagcaccccgcacaactactttctctagcagcagtgtcgctaacaatacaatttccaatccaacctttgccgcgcttttaaaacgcactgtgaataattctacaacttcacatacaacaatttccacttcaacaatcagcattatcgctgcagtgacaattggaatatctattcttgtttttaccataacctactacgcctgctgctatagaaaagacaaacataaaggtgatccattacttagatttgatatttaatttgttctttttttttttatttacagtatggtgaacaccaatcatggtacctagaaatttcttcttcaccatactcatttgtgcatttaatgtttgcgctactttcacagcagtagccacagcaaccccagactgtataggagcatttgcttcctatgcactttttgcttttgttacttgcatctgcgtatgtagcatagtctgcctggttattaattttttccaacttctagactggatccttgtgcgaattgcctacctgcgccaccatcccgaataccgcaaccaaaatatcgcggcacttcttagactcatctaaaaccatgcaggctatactaccaatatttttgcttctattgcttccctacgctgtctcaaccccagctgcctatagtactccaccagaacaccttagaaaatgcaaattccaacaaccgtggtcatttcttgcttgctatcgagaaaaatcagaaattcccccaaatttaataatgattgctggaataattaatataatctgttgcaccataatttcatttttgatataccccctatttgattttggctggaatgctcccaatgcacatgatcatccacaagacccagaggaacacattcccctacaaaacatgcaacatccaatagcgctaatagattacgaaagtgaaccacaacccccactactccctgctattagttacttcaacctaaccggcggagatgactgaaacactcaccacctccaattccgccgaggatctgctcgatatggacggccgcgtctcagaacagcgacttgcccaactacgcatccgccagcagcaggaacgcgcggccaaagagctcagagatgtcatccaaattcaccaatgcaaaaaaggcatattctgtttggtaaaacaagccaagatatcctacgagatcaccgctactgaccatcgcctctcttacgaacttggcccccaacgacaaaaatttacctgcatggtgggaatcaaccccatagttatcacccagcaaagtggagatactaagggttgcattcactgctcctgcgattccatcgagtgcacctacaccctgctgaagaccctatgcggcctaagagacctgctaccaatgaattaaaaaatgattaataaaaaatcacttacttgaaatcagcaataaggtctctgttgaaattttctcccagcagcacctcacttccctcttcccaactctggtattctaaaccccgttcagcggcatactttctccatactttaaaggggatgtcaaattttagctcctctcctgtacccacaatcttcatgtctttcttcccagatgaccaagagagtccggctcagtgactccttcaaccctgtctacccctatgaagatgaaagcacctcccaacacccctttataaacccagggtttatttccccaaatggcttcacacaaagcccagacggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaacggagggcttgttaatggctacgtgtctctagttggtgtatcagacactgtgaaccaaatgttcacacaaaagacagcaaacatccaattaagattatattttgactcttctggaaatctattaactgatgaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaaaataaagtttaagtgtttttatttaaaatcacaaaattcgagtagttattttgcctccaccttcccatttgacagaatacaccaatctctccccacgcacagctttaaacatttggataccattagagatagacattgttttagattccacattccaaacagtttcagagcgagccaatctggggtcagtgatagataaaaatccatcgcgatagtcttttaaagcgctttcacagtccaactgctgcggatgcgaatccggagtctggatcacggtcatctggaagaagaacgatgggaatcataatccgaaaacggtatcggacgattgtgtctcatcaaacccacaagcagccgctgtctgcgtcgctccgtgcaactgctgtttatgggatcagggtccacagtgtcctgaagcatgattttaatagcccttaacatcaactttctggtgcgatgcgcgcagcaacgcattctgatttcactcaaatctttgcagtaggtacaacacattattacaatattgtttaataaaccataattaaaagcgctccagccaaaactcatatctgatataatcgcccctgcatgaccatcataccaaagtttaatataaattaaatgacgttccctcaaaaacacactacccacatacatgatctcttttggcatgtgcatattaacaatctgtctgtaccatggacaacgttggttaatcatgcaacccaatataaccttccggaaccacactgccaacaccgctcccccagccatgcattgaagtgaaccctgctgattacaatgacaatgaagaacccaattctctcgaccgtgaatcacttgagaatgaaaaatatctatagtggcacaacatagacataaatgcatgcatcttctcataatttttaactcctcaggatttagaaacatatcccagggaataggaagctcttgcagaacagtaaagctggcagaacaaggaagaccacgaacacaacttacactatgcatagtcatagtatcacaatctggcaacagcgggtggtcttcagtcatagaagctcgggtttcattttcctcacaacgtggtaactgggctctggtgtaagggtgatgtctggcgcatgatgtcgagcgtgcgcgcaaccttgtcataatggagttgcttcctgacattctcgtattttgtatagcaaaacgcggccctggcagaacacactcttcttcgccttctatcctgccgcttagcgtgttccgtgtgatagttcaagtacagccacactcttaagttggtcaaaagaatgctggcttcagttgtaatcaaaactccatcgcatctaattgttctgaggaaatcatccacggtagcatatgcaaatcccaaccaagcaatgcaactggattgcgtttcaagcaggagaggagagggaagagacggaagaaccatgttaatttttattccaaacgatctcgcagtacttcaaattgtagatcgcgcagatggcatctctcgcccccactgtgttggtgaaaaagcacagctaaatcaaaagaaatgcgattttcaaggtgctcaacggtggcttccaacaaagcctccacgcgcacatccaagaacaaaagaataccaaaagaaggagcattttctaactcctcaatcatcatattacattcctgcaccattcccagataattttcagctttccagccttgaattattcgtgtcagttcttgtggtaaatccaatccacacattacaaacaggtcccggagggcgccctccaccaccattcttaaacacaccctcataatgacaaaatatcttgctcctgtgtcacctgtagcgaattgagaatggcaacatcaattgacatgcccttggctctaagttcttctttaagttctagttgtaaaaactctctcatattatcaccaaactgcttagccagaagccccccgggaacaagagcaggggacgctacagtgcagtacaagcgcagacctccccaattggctccagcaaaaacaagattggaataagcatattgggaaccgccagtaatatcatcgaagttgctggaaatataatcaggcagagtttcttgtaaaaattgaataaaagaaaaatttgccaaaaaaacattcaaaacctctgggatgcaaatgcaataggttaccgcgctgcgctccaacattgttagttttgaattagtctgcaaaaataaaaaaaaaaacaagcgtcatatcatagtagcctgacgaacaggtggataaatcagtctttccatcacaagacaagccacagggtctccagctcgaccctcgtaaaacctgtcatggtgattaaacaacagcaccgaaagttcctcgcggtgaccagcatgaataattcttgatgaagcatacaatccagacatgttagcatcagttaacgagaaaaaacagccaacatagcctttgggtataattatgcttaatcgtaagtatagcaaagccacccctcgcggatacaaagtaaaaggcacaggagaataaaaaatataattatttctctgctgctgttcaggcaacgtcgcccccggtccctctaaatacacatacaaagcctcatcagccatggcttaccagacaaagtacagcgggcacgcacaagctctaaagtcactctccaacctctccacaatatatatacacaagccctaaactgacgtaatgggagtaaagtgtaaaaaatcccgccaaacccaacacacaccccgaaactgcgtcaccagggaaaagtacagtttcacttccgcaatcccaacaagcgtcacttcctctttctcacggtacgtcacatcccattaacttgcaacgtcattttcccacggccgcgccgccccgtttagccgttaaccccacagccaatcaccacacaccccacaatttttaaaatcacctcatttacatattggcaccattccatctataaggtatattattgatgatg

(19) United States
(12) Patent Application Publication
(10) Pub. No.: US 2004/0106194 A1
(43) Pub. Date: Jun. 3, 2004

(54) Methods for propagating adenovirus and virus produced thereby

(76) Inventors: Andrew J. Bett, Lansdale, PA (US); Michael Chastain, Erdenheim, PA (US); Volker Sandig, Berlin (DE); Emilio A. Emini, Wayne, PA (US); John W. Shiver, Chalfont, PA (US); Danilo R. Casimiro, Harleysville, PA (US); David C. Kaslow, Rancho Santa Fe, CA (US); Manal Morsy, Blue Bell, PA (US)

Correspondence Address: MERCK AND CO INC, P O BOX 2000, RAHWAY, NJ, 070650907

(21) Appl. No.: 10/645,794

(22) Filed: Aug. 21, 2003

Related U.S. Application Data
(60) Provisional application No. 60/458,825, filed on Mar. 28, 2003.
(60) Provisional application No. 60/455,312, filed on Mar. 17, 2003.
(60) Provisional application No. 60/455,234, filed on Mar. 17, 2003.
(60) Provisional application No. 60/405,182, filed on Aug. 22, 2002.

(51) Int. Cl. C12Q 1/68; C12N 7/00; C12N 7/01; C12N 15/00; C12N 15/09; C12N 15/63; C12N 15/70; C12N 15/74

(52) U.S. Cl. 435/320.1; 435/235.1; 435/6

(57) ABSTRACT

Various methods for propagating and rescuing multiple serotypes of replication-defective adenovirus in a single adenoviral E1-complementing cell line are disclosed. Typically, replication-defective adenovirus vectors propagate only in cell lines which express E1 proteins of the same serotype or subgroup as the vector. The disclosed methods offer the ability to propagate vectors derived from multiple adenoviral serotypes in a single production cell line which expresses E1 proteins from a single serotype. Propagation in this manner is accomplished by providing all or a portion of an E4 region in cis within the genome of the replication-defective adenovirus. The added E4 region or portion thereof is cloned from a virus of the same or highly similar serotype as that of the E1 gene product(s) of the complementing cell line. Interaction between the expressed E1 of the cell line and the heterologous E4 of the replication-defective adenoviral vectors enables their propagation and rescue. The invention bypasses a need in the art to customize specific cell lines to the serotype or subgroup of the adenoviral vector being propagated and enables one to easily and rapidly develop alternative adenoviral serotypes as gene delivery vectors for use as vaccines or as a critical component in gene therapy.

### CROSS-REFERENCE TO RELATED APPLICATIONS

The present application claims the benefit of application serial No. 60/458,825, filed Mar. 28, 2003; No. 60/455,312, filed Mar. 17, 2003; No. 60/455,234, filed Mar. 17, 2003; and No. 60/405,182, filed Aug. 22, 2002.

### FIELD OF THE INVENTION

The present invention concerns various methods to propagate and rescue multiple serotypes of replication-defective adenovirus in a single adenoviral E1-complementing cell line. Typically, replication-defective adenovirus vectors propagate only in cell lines which express E1 proteins of the same serotype or subgroup as the vector. The methods disclosed herein offer the ability to propagate vectors derived from multiple serotypes in a single cell line expressing E1 proteins from a single serotype. Such propagation of a wide range of vectors in one cell line is accomplished by providing all or a portion of an E4 region in cis within the genome of the replication-defective adenovirus. The added E4 region or portion thereof is cloned from a virus of the same or highly similar serotype as that of the E1 gene product(s) of the complementing cell line. Interaction between the E1 gene products of the cell line and the heterologous E4 gene products of the replication-defective adenoviral vector enables the propagation and rescue of the recombinant replication-defective adenovirus vectors. The invention, therefore, bypasses an existing need in the art to customize complementing cell lines to the specific serotype or subgroup of the adenoviral vector being propagated or, alternatively, to have to transfect a cell line with an E4 region and then regulate the expression in trans of the E4 region within the E1 complementing cell line.

### BACKGROUND OF THE INVENTION

Beginning with the first human adenoviruses (Ads) isolated over four decades ago (Rowe et al., Proc. Soc. Exp. Biol. Med., 84:570-579, 1953), over 100 distinct serotypes of adenovirus have been isolated which infect various mammalian species, 51 of which are of human origin (Straus, Adenovirus infections in humans. In The Adenoviruses. 451-498, 1984; Hierholzer et al., J. Infect. Dis., 158: 804-813, 1988; Schnurr and Dondero, Intervirology., 36: 79-83, 1993; Jong et al., J Clin Microbiol., 37:3940-3945:1999). The human serotypes have been categorised into six subgenera (A-F) based on a number of biological, chemical, immunological and structural criteria; criteria which include hemagglutination properties of rat and rhesus monkey erythrocytes, DNA homology, restriction enzyme cleavage patterns, percentage of G+C content and oncogenicity (Straus, Adenovirus infections in humans. In The Adenoviruses. 451-498, 1984; Horwitz, Adenoviridae and their replication, In Virology: 1679-172, 1990).

Deletion of an essential E1 region common to the various adenovirus serotypes has enabled the use of adenovirus vectors as gene transfer vectors for vaccine and gene therapy purposes. Resultant replication-defective vectors are propagated in cell lines that provide the deleted E1 gene products in trans. Supplementation of the essential E1 gene products in trans in this manner works well when the E1 gene products are from the same or a highly similar serotype. As such, E1-deleted group C serotypes (Ad1, Ad2, Ad5 and Ad6) grow well in 293 or PER.C6 cells which contain and express the Ad5 E1 region. In contrast, E1-deleted serotypes other than group C, for example those from subgroups A, B, D, E, and F (e.g., Ad3, Ad4, and Ad7 to Ad51), do not replicate efficiently in 293 or PER.C6 cells. The Ad5 E1 sequences in 293 and PER.C6 cells do not fully complement the replication of these alternative serotypes. This presents a challenge due to the fact that the most characterized and studied complementing cell lines available for growth and propagation of adenovirus are based on E1 sequence from adenovirus serotype 5.

This inability to fully complement the replication of serotypes other than group C adenovirus in Ad15 E1 complementing cell lines has been attributed to the inability of Ad5 (group C) E1b 55K gene product to functionally interact with the E4 gene products of non-group C serotypes. While the interaction is conserved within members of the same subgroup, it is not well conserved between subgroups.

Hence, cell lines expressing both Ad5 E1 and ORF6 were generated and proved useful in complementing alternative adenovirus serotypes; see, e.g., Abrahamsen et al., 1997 J. Virol. 8946-8951. Such incorporation of E4 (or ORF6) into Ad 5 complementing cell lines as was done in Abrahamsen et al., supra, is known.

U.S. Pat. No. 5,849,561 discloses complementation of an E1-deleted non-group C adenovirus vector in an Ad5-E1 complementing cell line which also expresses portions of the Ad5-E4 gene.

U.S. Pat. No. 6,127,175, issued to Vigne, et al., discloses a stably transfected mammalian cell line which expresses a portion of the E4 region of adenovirus, preferably ORF6 or ORF6/7. Such a cell line is useful for complementation of recombinant Ad genomes deficient in the E4 region.

European Application EP 1 054 064 A1 discloses recombinant, replication deficient adenovirus 35 (Ad35) vectors and cell lines which complement in trans the growth of these vectors. A cell line which expresses Ad5E1A and E2A genes (PER.C6) was shown to complement an Ad35-E1 deleted vector upon co-expression of Ad35-E1B proteins.

U.S. Pat. No. 6,270,996, issued to Wilson, et al., discloses E1/E4 deleted adenovirus vectors and E1/E4 (ORF6) cell lines which complement in trans virus growth without resulting in cell toxicity.

U.S. Pat. No. 6,202,060, issued to Mehtali, et al., discloses adenoviral vectors wherein portions of the early genes are under control of an inducible promoter. The '060 patent also discloses complementing cell lines which may be used in tandem with these Ad vectors.

The generation of serotype-specific cell lines providing a complementing serotype-specific E1 gene product(s) in trans is known as well.

Although Ad5-based vectors have been used extensively in a number of gene therapy trials, there may be limitations on the use of Ad5 and other group C adenoviral vectors due to preexisting immunity in the general population due to natural infection. Ad5 and other group C members tend to be among the most seroprevalent serotypes. Immunity to existing vectors may develop as a result of exposure to the vector during treatment. These types of preexisting or developed immunity to seroprevalent gene delivery vectors may limit the effectiveness of gene therapy or vaccination efforts. Alternative adenovirus serotypes, thus, constitute very important targets in the pursuit of gene delivery systems capable of evading the host immune response.

There remains both a practical and commercial need for an adenovirus-based vaccine and/or gene therapy delivery system which allows for the production of multiple serotype recombinant adenovirus vectors in a single source complementing mammalian cell line. The present invention addresses and overcomes this deficiency in the art by disclosing novel methods for propagating multiple serotype recombinant Ad vectors in a single complementing cell line where the required serotype-specific sequences are provided in cis.

### SUMMARY OF THE INVENTION

The present invention relates to an enhanced means for propagating replication-defective adenovirus in an E1-complementing cell line(s) where the E1 gene product(s) being expressed is not native to the adenovirus being propagated. The method is based on Applicants' finding that supply, in cis, of a nucleic acid sequence encoding all or a portion of a heterologous adenoviral E4 region which is native to a virus of the same or highly similar serotype as the E1 gene product(s) of the complementing cell line enables the growth of adenoviral vectors of varying serotype in any single complementing cell line, despite the fact the cell line is not customized for the particular serotype of vector being propagated. This is of particular importance given that existing and settled adenoviral E1-complementing cell lines (such as PER.C6™ and 293) are based on one of the most prominent adenovirus serotypes (Ad5) and are not suited for the large-scale propagation and rescue of alternative serotypes.

The basic steps involved in the propagation of adenoviral vectors in accordance with the methods of the instant invention are as follows: First, all or a portion of a heterologous adenoviral E4 region comprising nucleic acid sequence encoding at least open reading frame 6 (ORF6) is inserted into a replication-defective adenoviral vector. By “heterologous”, Applicants mean that the nucleic acid sequence is not native to the viral vector being propagated, i.e., not normally present within a virus of the same or highly similar serotype. As will be described, the adenoviral E4 region or portion thereof can be either a nucleic acid sequence encoding ORF 6 or any larger portion of the E4 region, and includes nucleic acid comprising the complete E4 region with E4 promoter. The region into which the nucleic acid is incorporated is not limited, i.e., the insertion can be made into the complete E4 region with E4 promoter or into a smaller portion narrowing into the ORF6 region. Alternatively, the heterologous E4 region or portion thereof can be inserted into different areas of the genome such as the E1 or E3 regions. Further, the native E4 region or portion thereof can be deleted and replaced, or left intact. This is not deemed a critical element of the instant invention. What is a critical element is that the heterologous E4 region or portion thereof being inserted is native to a virus of the same or highly similar serotype as the E1 gene product(s) expressed by the complementing cell line.

Following the modification of the adenoviral vector of interest, the recombinant adenovirus is then introduced into an adenoviral E1-complementing cell line and allowed to propagate. The adenovirus is subsequently harvested and rescued from the complementing cell line.

The resultant virus can be studied and used in various gene therapy and vaccine efforts. The virus, therefore, forms an important aspect of the instant invention.

### BRIEF DESCRIPTION OF THE DRAWINGS

FIG. 1 illustrates a transcription map for adenovirus serotype 5. The linear genome is divided into 100 map units as well as into r- and l-strands which designate the direction of transcription. Early transcription units are designated with an E and are active prior to viral DNA replication. Late transcription units are designated with and L and are active primarily after DNA replication. Promoters are represented as brackets and polyadenylation sites as arrowheads. The tripartite leader is designated 1, 2, and 3.

FIGS. 2A-1 through 2A-10 illustrate the nucleic acid sequence for the wild-type adenovirus 35 (SEQ ID NO: 1) utilized in the Examples.

FIG. 3 illustrates the homologous recombination scheme utilized to recover pAd35ΔE1.

FIG. 4 illustrates the various configurations of the E4 regions (or portions) within the alternative serotype recombinants.

FIG. 5 illustrates the homologous recombination scheme utilized to recover pAd35ΔE1ΔE4Ad5Orf6.

FIG. 6 illustrates the nucleic acid sequence encoding the gag expression cassette (SEQ ID NO: 2). The various regions of the figure are as follows: (1) a first underlined segment of nucleic acid sequence encoding the immediate early gene promoter region from human cytomegalovirus; (2) a first segment of lowercase letters which is not underlined, which segment of DNA contains a convenient restriction enzyme site; (3) a region in caps which contains the coding sequence of HIV-1 gag; (4) a second segment of lowercase letters which is not underlined, which segment of DNA contains a convenient restriction enzyme site; and (5) a second underlined segment, this segment containing nucleic acid sequence encoding a bovine growth hormone polyadenylation signal sequence.

FIG. 7 illustrates the nucleic acid sequence encoding the SEAP expression cassette (SEQ ID NO: 3). The various regions of the figure are as follows: (1) a first underlined segment of nucleic acid sequence encoding the immediate early gene promoter region from human cytomegalovirus; (2) a first segment of lowercase letters which is not underlined, which segment of DNA contains a convenient restriction enzyme site; (3) a region in caps which contains the coding sequence of the human placental SEAP gene; (4) a second segment of lowercase letters which is not underlined, which segment of DNA contains a convenient restriction enzyme site; and (5) a second underlined segment, this segment containing nucleic acid sequence encoding a bovine growth hormone polyadenylation signal sequence.

FIG. 8 illustrates in vivo expression of SEAP in C3H/HeN mice using 10\hat{ }10 vp doses of Ad35 vectors. This experiment was designed to address any effects of E3 deletion. The vectors were injected intramuscularly and the levels of SEAP expression were determined from the serum samples. Shown are geometric means for each cohort of 5 mice.

FIG. 9 illustrates in vivo expression of SEAP in C3H/HeN mice using 10\hat{ }10 vp doses of Ad35 vectors. This experiment was designed to address any effects of Ad5 sequence insertion into the Ad35 genome. The vectors were injected intramuscularly and the levels of SEAP expression were determined from the serum samples. Two extra cohorts received 10\hat{ }10 vp and 10\hat{ }9 vp of Ad5 vector. Shown are geometric means for each cohort of 5 mice.

FIGS. 10A-B illustrate in vivo SEAP expression using MRKAd5-based (A) and Ad35ΔE1ΔE4Ad5Orf6-based (B) vector in rhesus macaques. Shown are the serum antigen levels for individual monkeys following a single intramuscular (i.m.) injection of 10\hat{ }11 vp MRKAd5SEAP (filled circles), 10\hat{ }9 vp MRKAd5SEAP (open boxes) or 10\hat{ }11 vp Ad35ΔE1SEAPΔE4Ad5Orf6.

FIG. 11 illustrates in vivo SEAP expression in African green monkeys using Ad5- and Ad35-based vectors. Shown are the antigen levels for each animal in serum samples collected two days after the treatment.

FIG. 12 illustrates the homologous recombination scheme utilized to recover pAd24ΔE1.

FIG. 13 illustrates the homologous recombination scheme utilized to recover pAd24ΔE1Ad5Orf6.

FIG. 14 illustrates the configuration of E4 regions in the Ad24 recombinants generated.

FIG. 15 illustrates the growth kinetics of the Ad24-based vectors in PER.C6 cells.

FIGS. 16A-1 through 16A-10 illustrate the nucleic acid sequence for wild-type adenovirus serotype 24 (SEQ ID NO: 5). The ATCC product number for Ad24 is VR-259.

FIG. 17 illustrates, in tabular format, gag-specific T cell responses in monkeys immunized with MRKAd5-HIVgag and Ad24 HIV vectors. Shown are the numbers of spot-forming cells per million PBMC following incubation in the absence (mock) or presence of Gag peptide pool. The pool consisted of 20-aa peptide overlapping by 10 aa and encompassing the entire gag sequence.

FIG. 18 illustrates, in tabular format, the characterization of the gag-specific T cells in monkeys immunized with 10\hat{ }11 vp of MRKAd5-HIV1gag and Ad24ΔE1gagΔOrf6Ad5Orf6. Shown are the percentages of CD3+ T cells that are either gag-specific CD4+ or gag-specific CD8+ cells. These values were corrected for mock values (<0.03%).

FIG. 19 illustrates individual anti-p24 titers (in mMU/mL) in macaques immunized with gag-expressing adenovirus vectors.

FIG. 20 illustrates in vivo expression of SEAP in C3H/HeN mice using 10\hat{ }10 vp doses of Ad24 vectors. The vectors were injected intramuscularly and the levels of SEAP expression were determined from the serum samples. Two extra cohorts received 10\hat{ }10 vp and 10\hat{ }9 vp of Ad5 vector. Shown are geometric means for each cohort of 5 mice.

FIG. 21 illustrates in vivo SEAP expression using MRKAd5 and Ad24 vectors in rhesus macaques. Shown are the geometric means of the SEAP levels for cohorts of 3 monkeys. In bars are the standard errors of the geometric means.

FIG. 22 illustrates a homologous recombination scheme to be utilized to recover pAd24ΔE1ΔE4Ad5Orf6.

FIG. 23 illustrates gag-specific T cell responses in rhesus macaques immunized following a heterologous Ad5/Ad6 prime-Ad24 boost regimen. a: Mock, no peptide: gag, 20-mer peptide pool encompassing entire gag sequence; b: Peak response after 2 or 3 doses of the priming vaccine; c: 3 wks prior to boost; d: 4 wks after boost; e: ND, not determined.

FIG. 24 illustrates, in tabular format, the percentages of CD3⁺ T lymphocytes that are gag-specific CD8⁺ cells or gag-specific CD4⁺ cells determined after the Ad24 Boost Immunization (wk 60). Numbers reflect the percentages of circulating CD3+ lymphocytes that are either gag-specific CD4+ or gag-specific CD8+ cells. Mock values (equal to or less than 0.01%) have been subtracted.

FIG. 25 illustrates gag-specific T cell responses in rhesus macaques immunized following a heterologous Ad 24 prime-Ad5 boost regimen. a: Mock, no peptide: gag, 20-mer peptide pool encompassing entire gag sequence; b: Peak response after 2 doses of the priming vaccine; c: Wk 24; d: 4 wks after boost; e: ND, not determined.

FIG. 26 illustrates the homologous recombination scheme utilized to recover pAd34ΔE1ΔE4Ad5Orf6.

FIG. 27 illustrates the homologous recombination scheme utilized to recover pMRKAd34ΔE1ΔE4Ad5Orf6.

FIGS. 28A-1 to 28A-9 illustrate a nucleic acid sequence for wild-type adenovirus serotype 34 (SEQ ID NO: 12). The ATCC product number for Ad34 is VR-716.

FIG. 29 illustrates the time course of SEAP expression using MRKAd5 and Ad34 vectors in rhesus macaques. Data represent cohort geometric means.

FIG. 30 illustrates, in tabular format, T cell responses induced using MRKAd5 and Ad34 vectors expressing HIV-1 gag. Data are expressed in numbers of spot-forming cells per million PBMC (SFC/10\hat{ }6 PBMC). “a” refers to a 20-mer peptide pool with 10-aa overlap and encompassing the entire HIV-1 CAM1 gag.

FIG. 31 illustrates, in tabular format, the levels of CD4+ and CD8+ Gag-specific T cells in Ad34-immunized macaques at week 12. “a” refers to a 20-mer peptide pool with 10-aa overlap and encompassing the entire HIV-1 CAM1 gag.

FIG. 32 illustrates, in tabular format, T cell responses induced using a heterologous Ad34 prime/Ad35 boost regimen in macaques. “a” refers to a 20-mer peptide pool with 10-aa overlap and encompassing the entire HIV-1 CAM1 gag.

FIG. 33 illustrates, in tabular format, the levels of CD4+ and CD8+ Gag-specific T cells in Ad34 primed/Ad35 boosted macaques at week 28. “a” refers to a 20-mer peptide pool with 10-aa overlap and encompassing the entire HIV-1 CAM1 gag.

### DETAILED DESCRIPTION OF THE INVENTION

The present invention details an efficient strategy for the propagation and rescue of alternative adenoviral serotypes utilizing available adenovirus production cell lines, nullifying the need to customize available cell lines for a specific serotype of interest. This is enabled by the incorporation of a critical E4 region into the adenovirus to be propagated.

The critical E4 region in the instant invention comprises, in the minimum, nucleic acid sequence encoding E4 ORF6 and can comprise the entire region of E4, inclusive of the promoter region. An important characteristic of the imported E4 region is that it is native to a virus of the same or highly similar serotype as the E1 gene product(s) (particularly E1B 55K) of the E1-complementing cell line, but heterologous to (i.e., non-native to a virus of the same serotype as) the adenoviral vector being propagated. As will be detailed below, the heterologous E4 region or portion thereof can be varied and can be inserted into the vector backbone at numerous locations.

The heterologous E4 region or portion thereof can, for instance, be a nucleic acid sequence encoding the entire open reading frame of the non-native E4. This segment of nucleic acid sequence can, in turn, be incorporated into the “native” entire E4 open reading frame of the recipient virus. In such an embodiment, the promoter native to the adenoviral vector would drive the expression of the non-native E4 region within the recombinant replication-defective adenoviral vector. Alternatively, the nucleic acid sequence encoding the entire open reading frame can be inserted into a different region of the adenoviral vector genome, such as for example the E1 or E3 regions. In this latter embodiment, the native E4 region or portion thereof can be deleted or left intact.

In another embodiment, the heterologous E4 region comprises a nucleic acid sequence encoding the entire open reading frame of E4 and includes a non-native E4 promoter. In this type of embodiment, the E4 region can be inserted into the location of the combined native E4 and E4 promoter region. The non-native E4 region in this embodiment would be driven by expression of the non-native E4 promoter. Alternatively, the nucleic acid sequence encoding the entire open reading frame and the non-native E4 promoter can be inserted into a different region of the adenoviral vector genome, such as for example the E1 or E3 regions. In this latter embodiment, the native E4 region or portion thereof can be deleted or left intact.

An alternative and further embodiment exists wherein the heterologous E4 region or portion thereof comprises nucleic acid sequence encoding a partial E4 region comprising ORF6 (one aspect of which is a region solely encoding ORF6). In this particular aspect of the invention, the heterologous non-native E4 protein can, in certain embodiments, replace the nonnative ORF6 region or the entire E4-encoding region of the native virus. In the latter situation, the promoter driving expression of the non-native ORF6 can either be the native E4 promoter or a heterologous, non-native promoter operatively linked to the non-native ORF6, while in the latter, the expression of the non-native ORF6 would generally be driven by the native E4 promoter. Alternatively, the nucleic acid sequence encoding a partial E4 region comprising ORF 6 can be inserted into a different region of the adenoviral vector genome, such as for example the E1 or E3 regions. In this latter embodiment, the native E4 region or portion thereof can be deleted or left intact.

As one of skill in the art can appreciate, there are various ways in which one can envision the supply of a heterologous E4 nucleic acid sequence in cis to an adenoviral vector and thereby enable its growth based on Applicants' novel findings herein. Moreover, as one of skill in the art can appreciate, either native or non-native promoters can be utilized to drive expression of the heterologous E4 region or portion thereof.

Adenovirus pre-plasmids (plasmids comprising the genome of the replication-defective adenovirus with desired deletions and insertions) can be generated by homologous recombination using adenovirus backbones and an appropriate shuttle vector (designed to target-in specific deletions and incorporate desired restriction sites into the resultant plasmid). Shuttle vectors of use in this process can be generated using general methods widely understood and appreciated in the art, e.g., PCR of the adenoviral terminal ends taking into account the desired deletions, and the sequential cloning of the respective segments into an appropriate cloning plasmid. The adenoviral pre-plasmid can then be digested and transfected into the complementing cell line via calcium phosphate co-precipitation or other suitable means. Virus replication and amplification then occurs, a phenomenon made evident by notable cytopathic effect. Infected cells and media are then harvested after viral replication is complete (generally, 7-10 days post-transfection).

It is to be noted that various alternative adenoviral serotypes can be developed in accordance with the disclosed methods and, particularly, alternative adenoviral serotype vectors that were previously unable to be propagated or very inefficiently propagated utilizing existing adenoviral production cell lines based on subgroup C complementing E1 sequence. The various adenoviral vectors that can be developed in accordance with the instant methods include adenoviral vectors of subgroups A-F (for instance, serotypes of subgroups A, B (e.g., serotypes 11, 14, 16, 21, 34 and 35), C (e.g., serotypes 2 and 5), D (e.g., serotypes 24, 26 and 36), E (e.g., serotype 4) and F.

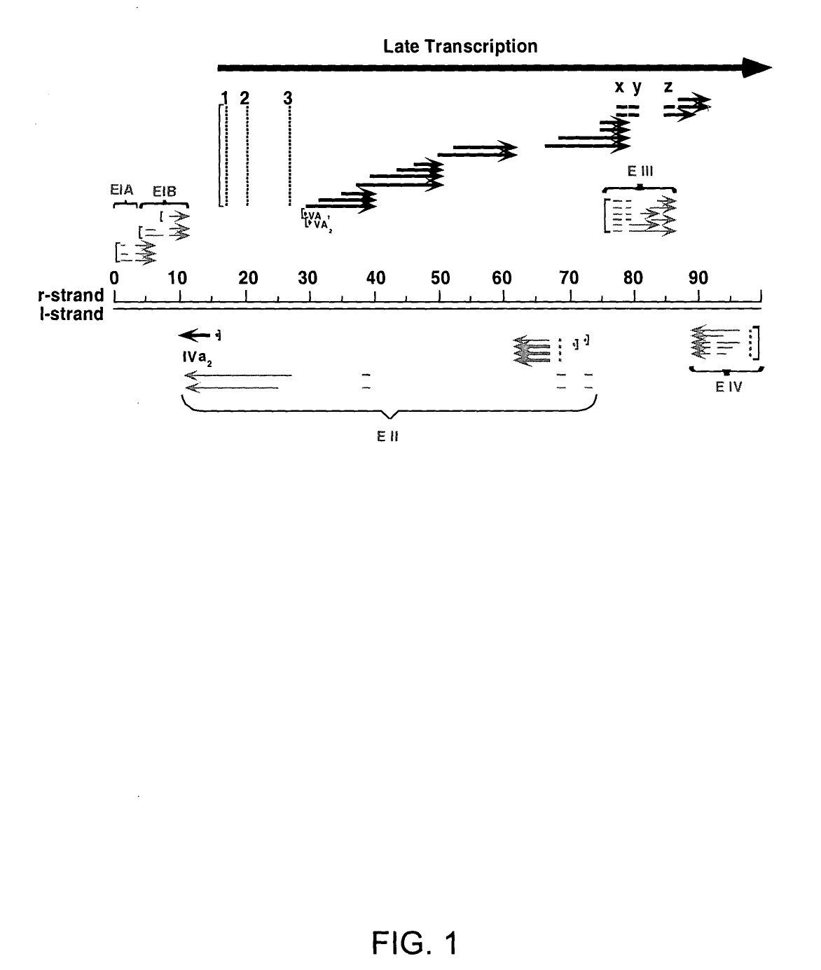

In preferred embodiments, the various non-group C family members can be developed with heterologous E4 supplied from a subgroup C member such as adenovirus serotype 5. Particular embodiments of the instant invention utilize a development scheme wherein the heterologous E4 protein is derived from a wildtype adenovirus serotype 5 sequence; see, e.g., a viral sequence which has been deposited with the American Type Culture Collection (“ATCC”) under ATCC Deposit No. VR-5 (for which a transcription map can be found in FIG. 1). A particular example of this type of embodiment is wherein an adenovirus of subgroup B (or any non-C subgroup) comprising heterologous E4 proteins in cis from Ad5 is propagated in Ad5 E1-complementing cell lines, for instance, PER.C6™ or 293. Applicants have, in fact, successfully propagated E1-serotypes 10, 24, 34, and 35 via use of this particular embodiment.

One of skill in the art can readily identify alternative adenovirus serotypes (e.g., alternative serotypes of subgroups A, B (e.g., serotypes 11, 14, 16, 21, 34 and 35), C, (e.g., serotypes 2 and 5), D (e.g., serotypes 24, 26 and 36), E (e.g., serotype 4) and F) for the supply of the heterologous E4 protein. As long as the heterologous E4 region (or portion thereof comprising ORF6) of the vector is native to a virus of the same or highly similar serotype as the E1 region of the complementing cell line, the methods of the instant invention are widely applicable to the propagation and rescue of adenovirus of all serotypes. In light of the present disclosure, one can readily envision, for instance, how a complementing cell line based on a non-subgroup C adenovirus (e.g., the Ad35 cell line of EP 1 054 064 A1) can be utilized to propagate a virus of an adenoviral vector of subgroup C (e.g., adenovirus serotype 5) provided that the appropriate nucleic acid sequence encoding an E4 protein provided in cis is native to a virus of the same or highly similar serotype as that of the E1 expressed by the complementing cell line (i.e., an Ad35 E4 protein).

Complementing cell lines of use in the instant invention are available in the art and are not limited to any specific type. The critical feature, again, is that the heterologous segment of E4-encoding nucleic acid sequence provided in cis to the replication-defective vector being propagated be native to a virus of the same or highly similar serotype as the E1 expressed by the complementing cell line. One aspect of the instant invention employs E1-complementing cell lines wherein the expressed E1 is of serotype 5; e.g., PER.C6™ and 293 cell lines. Both these cell lines express the adenoviral E1 gene product. PER.C6™ is described in Fallaux et al., 1998 Human Gene Therapy 9:1909-1917, hereby incorporated by reference. 293 cell lines are described in Graham et al., 1977 J. Gen. Virol. 36:59-72, hereby incorporated by reference.

Another aspect of the instant invention are the adenoviral vectors of any serotype falling with adenoviral subgroups A, B, C, D, E and F (for instance, alternative serotypes of subgroups A, B (e.g., serotypes 11, 14, 16, 21, 34 and 35), C (e.g., serotype 2), D (e.g., serotypes 24, 26 and 36), E (e.g., serotype 4) and F) which are modified to contain a non-native E4-encoding nucleic acid sequence in cis which comprises, in whole or in part, nucleic acid sequence encoding open reading frame 6 (ORF6). Virus in accordance with this description can be propagated in accordance with the above-described methods and rescued using any suitable means known in the art.

Another aspect of the instant invention is a vector in accordance with the instant invention which comprises a heterologous passenger gene in addition to that of the heterologous E4 nucleic acid sequence. In specific embodiments, the passenger gene encodes an antigen.

As one of ordinary skill in the art will appreciate, the instant methods are not limited by the heterologous gene that can be incorporated. The instant invention relates generally to a means by which to propagate multiple serotypes of adenovirus in a single complementing cell line and the recombinant virus that make the process possible. In preferred embodiments, the passenger gene is incorporated into the E1 deletion. In alternatively preferred embodiments, the passenger gene is inserted in an E3-deleted region. The position of the passenger gene, as one of ordinary skill in the art will appreciate, can be varied according to the specific complementing cell utilized and the specific deletions present within the replication-defective adenovirus genome.

In specific embodiments the passenger gene can encode an HIV-1 antigen, and in more preferred embodiments selected from the group consisting of genes encoding HIV-1 gag, pol, nef and env. In alternative embodiments, the passenger gene can be a reporter gene, such as secreted alkaline phosphatase (SEAP).

The passenger gene preferably exists in the form of an expression cassette. A gene expression cassette preferably comprises (a) a nucleic acid sequence encoding a protein of interest; (b) a promoter operatively linked to the nucleic acid sequence encoding the protein; and (c) a transcription termination sequence. The transcriptional promoter of the adenoviral vector is preferably recognized by an eukaryotic RNA polymerase. In a preferred embodiment, the promoter is a “strong” or “efficient” promoter. An example of a strong promoter is the immediate early human cytomegalovirus promoter (Chapman et al., 1991 Nucl. Acids Res. 19:3979-3986), which is hereby incorporated by reference), in certain embodiments without intronic sequences. Those skilled in the art, however, will appreciate that any of a number of other known promoters, such as the strong immunoglobulin, or other eukaryotic gene promoters may also be used, including the EF1 alpha promoter, the murine CMV promoter, Rous sarcoma virus (RSV) promoter, SV40 early/late promoters and the beta-actin promoter.

The promoter may comprise a regulatable sequence such as the Tet operator sequence. This is extremely useful, for example, in cases where the gene products are affecting a result other than that desired and repression is sought.

Transcription termination sequences can also be utilized within the gene expression cassettes. Preferred termination sequences are, for instance, the bovine growth hormone terminator/polyadenylation signal (bGHpA) and the short synthetic polyA signal (SPA) of 50 nucleotides in length, defined as follows: AATAAAAGATCTTTATTTTCATTAGATCTGTGTGTTGGT-TTTTTGTGTG (SEQ ID NO:4).

Further embodiments incorporate a leader or signal peptide into the transgene. A preferred leader is that from the tissue-specific plasminogen activator protein, tPA.

The following non-limiting Examples are presented to better illustrate the invention.

### EXAMPLE 1

Construction and Rescue

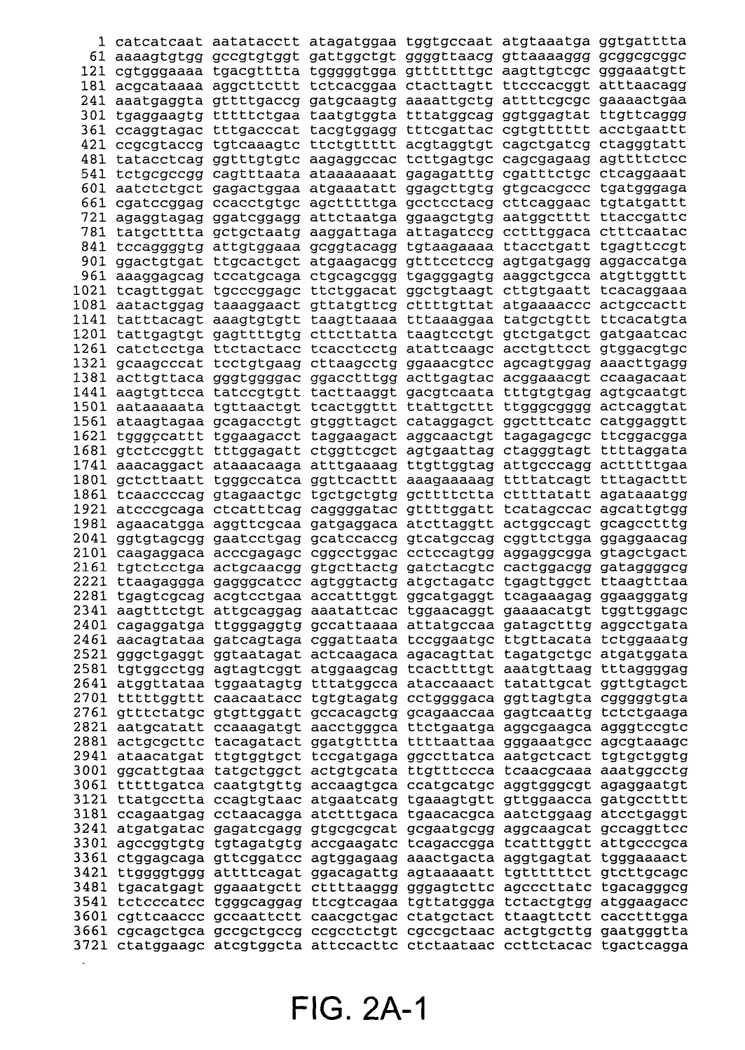

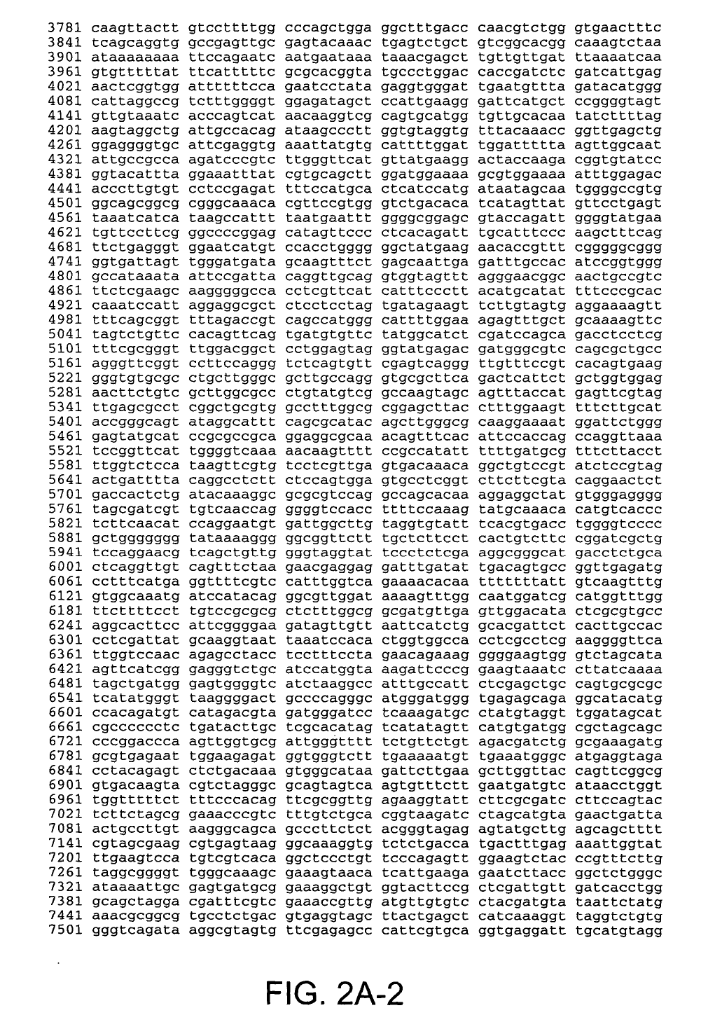

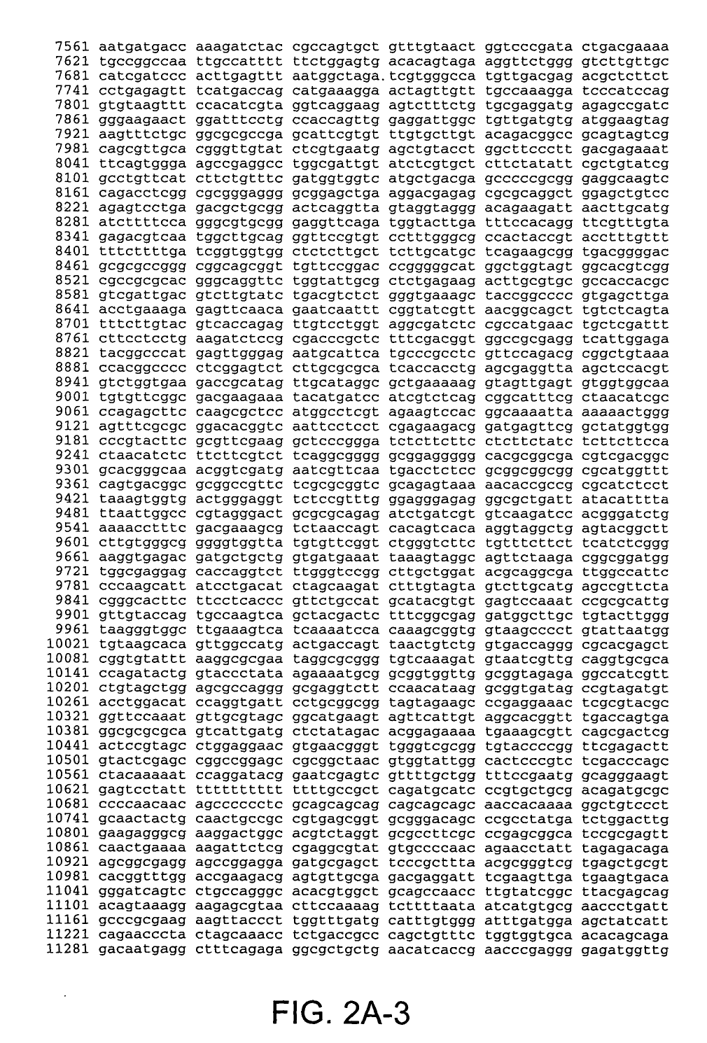

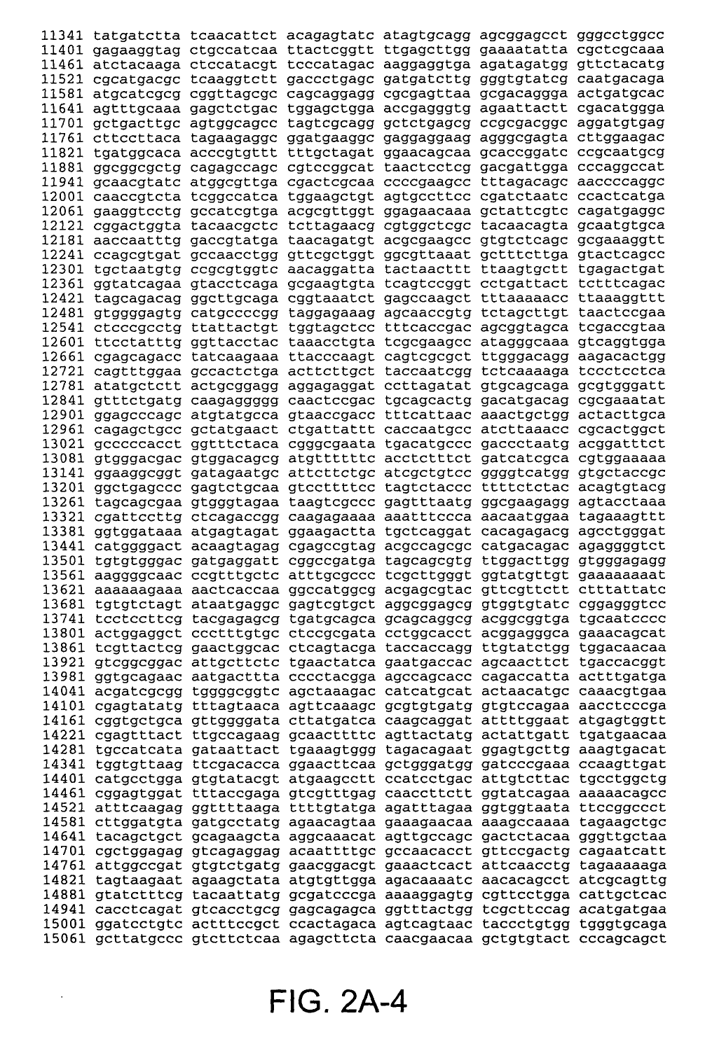

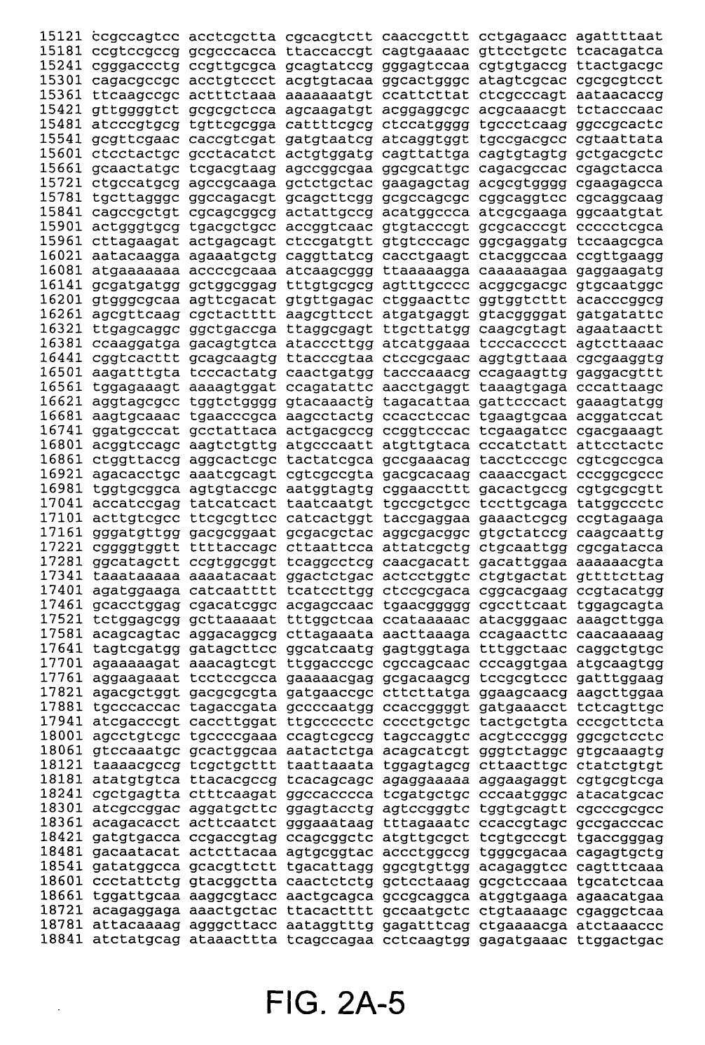

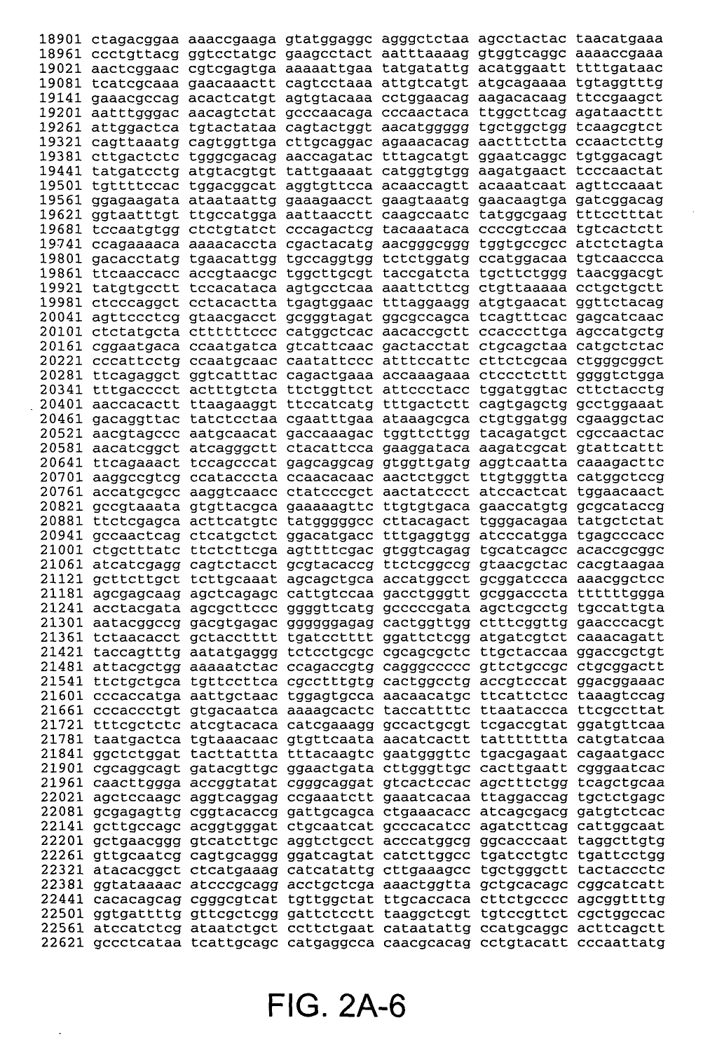

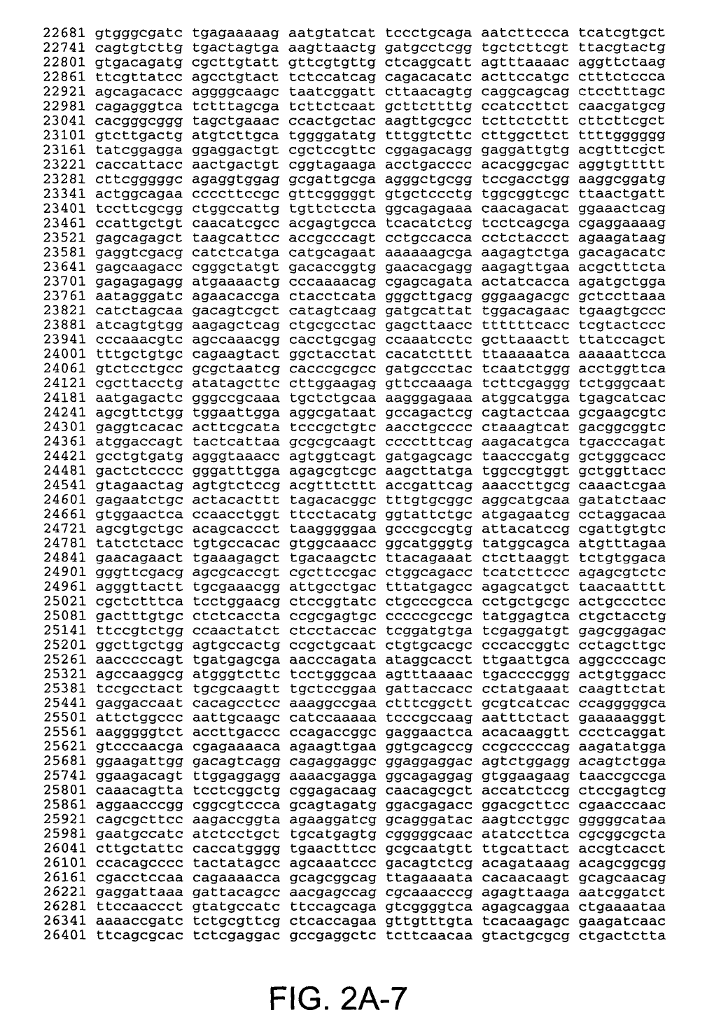

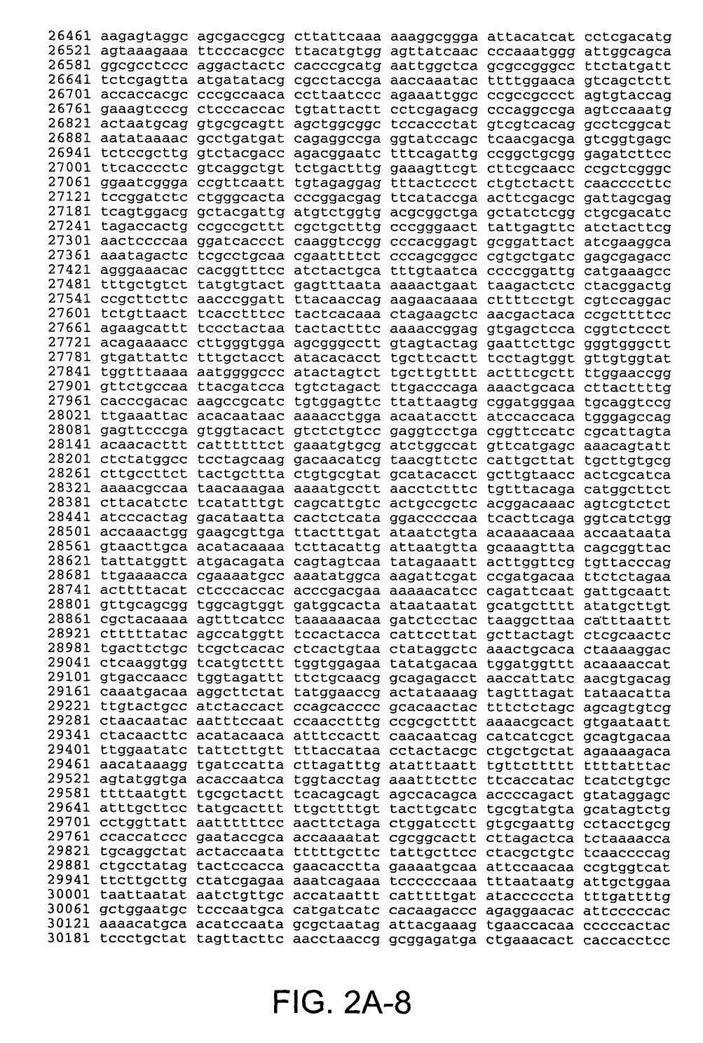

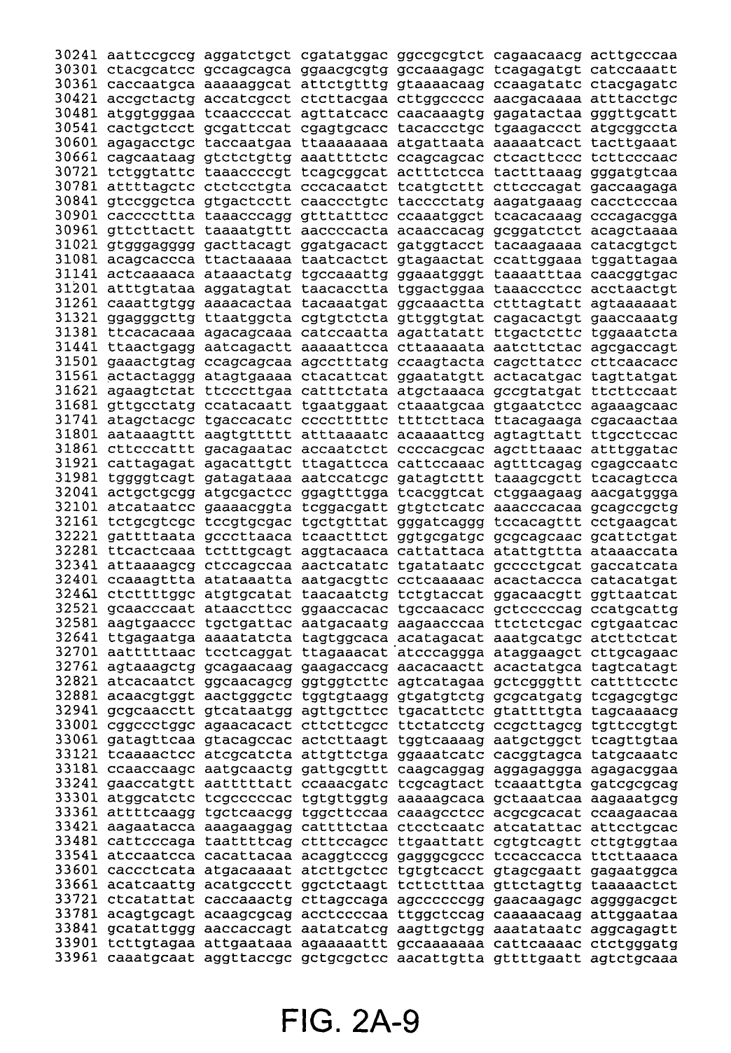

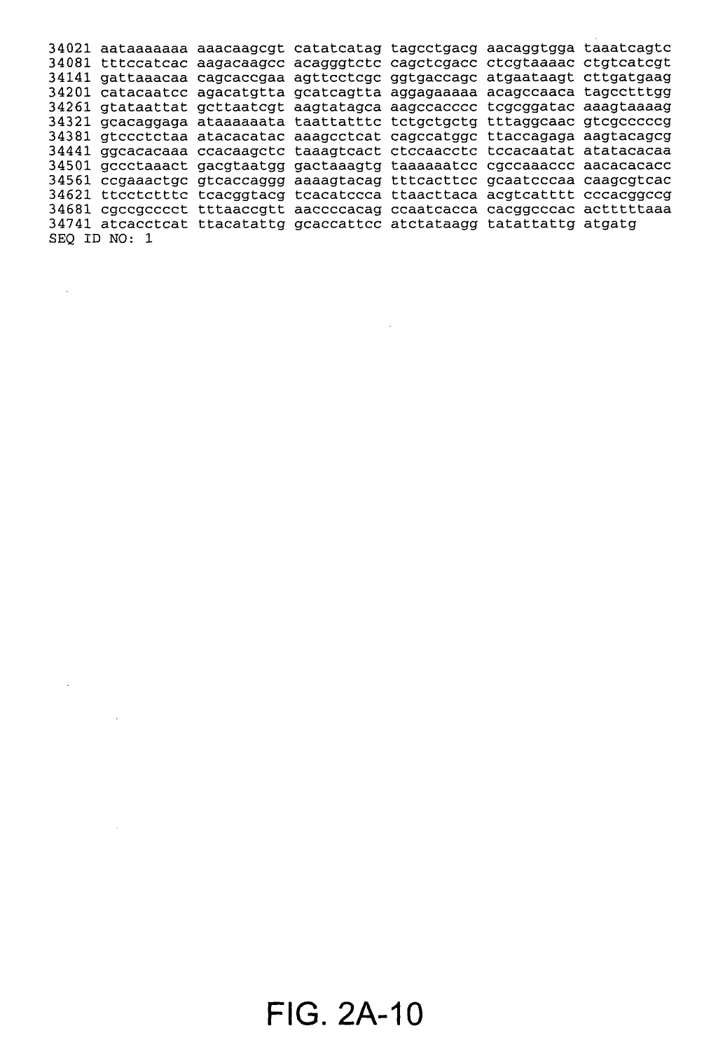

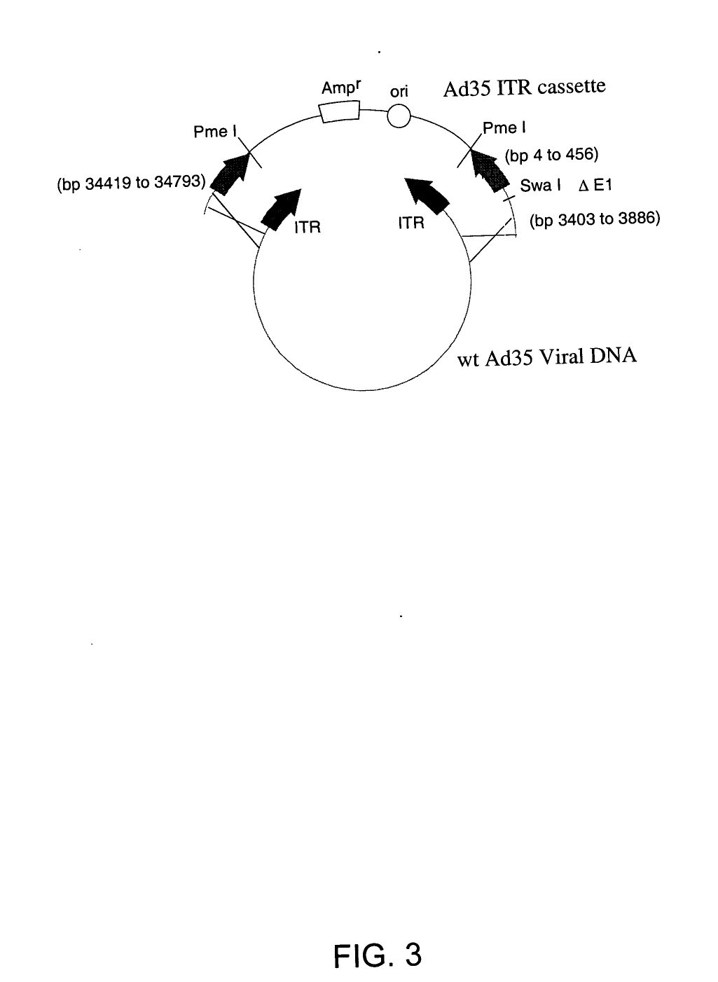

An E1-Ad35-based pre-adenovirus plasmid was constructed in order to determine whether an E1-Ad35 vector (a representative group B serotype) could be propagated in a group C E1-complementing cell line. The general strategy used to recover Ad35 as a bacterial plasmid is illustrated in FIG. 3. Cotransformation of BJ5183 bacteria with purified wild-type Ad35 viral DNA and a second DNA fragment termed the Ad35 ITR cassette resulted in the circularization of the viral genome by homologous recombination. The ITR cassette contains sequences from the right (bp 34419 to 34793) and left (bp 4 to 456 and bp 3403 to 3886) end of the Ad35 genome (see FIGS. 2A-1 to 2A-10) separated by plasmid sequences containing a bacterial origin of replication and an Ampicillin resistance gene. The ITR cassette contains a deletion of E1 sequences from Ad5 457 to 3402 with a unique Swa I site located in the deletion. The Ad35 sequences in the ITR cassette provide regions of homology with the purified Ad35 viral DNA in which recombination can occur. The ITR cassette was also designed to contain unique restriction enzyme sites (Pme I) located at the end of the viral ITR's so that digestion will release the Ad35 genome from plasmid sequences. Potential clones were screened by restriction analysis and one clone was selected as pAd35ΔE1. Pre-Adenovirus plasmid pAd35ΔE1 contains Ad35 sequences from 4 to 456 and bp 3403 to 34793.

To determine if pre-adenovirus plasmid pAd35ΔE1 could be rescued into virus and propagated in a group C E1 complementing cell line, the plasmid was digested with Pme I and transfected into a T-25 flask of PER.C6 cells using the calcium phosphate co-precipitation technique. Pme I digestion releases the viral genome from the plasmid sequences allowing viral replication to occur after entry into 293 cells. Viral cytopathic effect (CPE), indicating that virus replication and amplification is occurring, was never observed. Cells and media from the transfection were harvested at 14 days post transfection, freeze-thawed three times, clarified by centrifugation and used to infect new PER.C6 cells but no virus was ever amplified. Following multiple attempts, we have been unable to rescue and amplify pAd35ΔE1 in PER.C6 cells.

### EXAMPLE 2

Insertion of Ad5 Orf 6 and Ad5 E4 into the Ad5 Genome

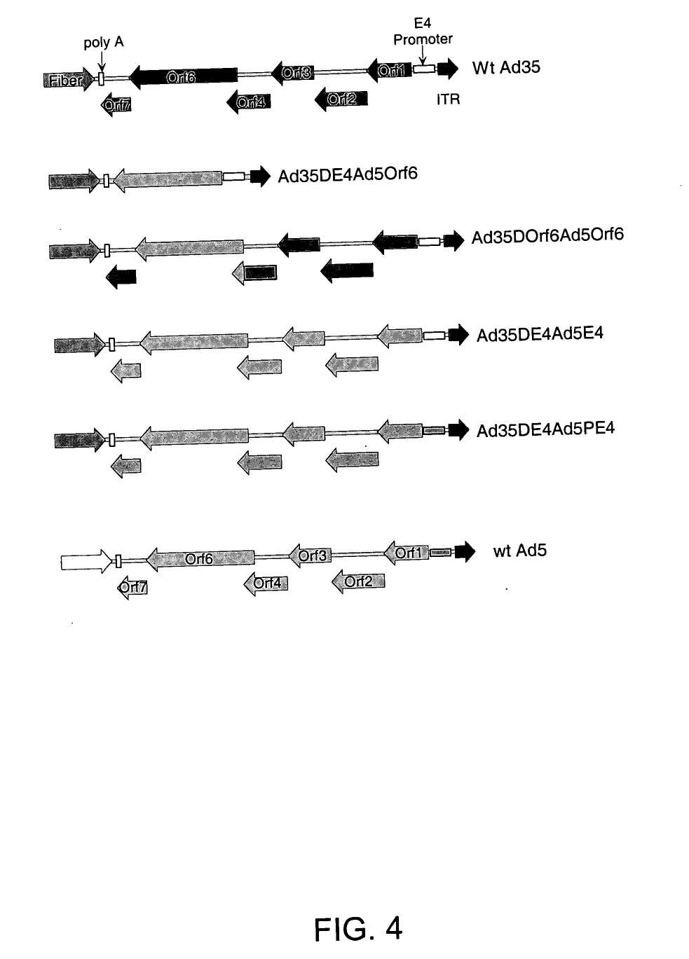

To refine the strategy of including Ad5 Orf6 in the genome of an alternative serotype so that propagation could take place in a Ad5/group C complementing cell line four additional strategies were developed. In the first strategy, the entire alternative serotype E4 region (not including the E4 promoter) was deleted and replaced with Ad5 Orf6. In the second strategy, just the alternative serotype Orf6 gene was deleted and replaced with Ad5 Orf6. In the third strategy, the entire alternative serotype E4 coding region (not including the E4 promoter) was deleted and replaced with the Ad5 E4 coding region (not including the Ad5 E4 promoter) and, in the final strategy, the entire alternative serotype E4 coding and promoter region was deleted and replaced with the Ad5 E4 promoter and coding region. The configuration of the E4 regions generated by the four strategies is diagramed in FIG. 4. For each of these strategies the desired pre-Adenovirus plasmid was generated by bacterial recombination. Cotransformation of BJ 5183 bacteria with purified wild-type viral DNA and the appropriately constructed ITR cassette resulted in the circularization of the viral genome by homologous recombination. The construction of each pre-Ad plasmid, based on Ad35, is outlined below:

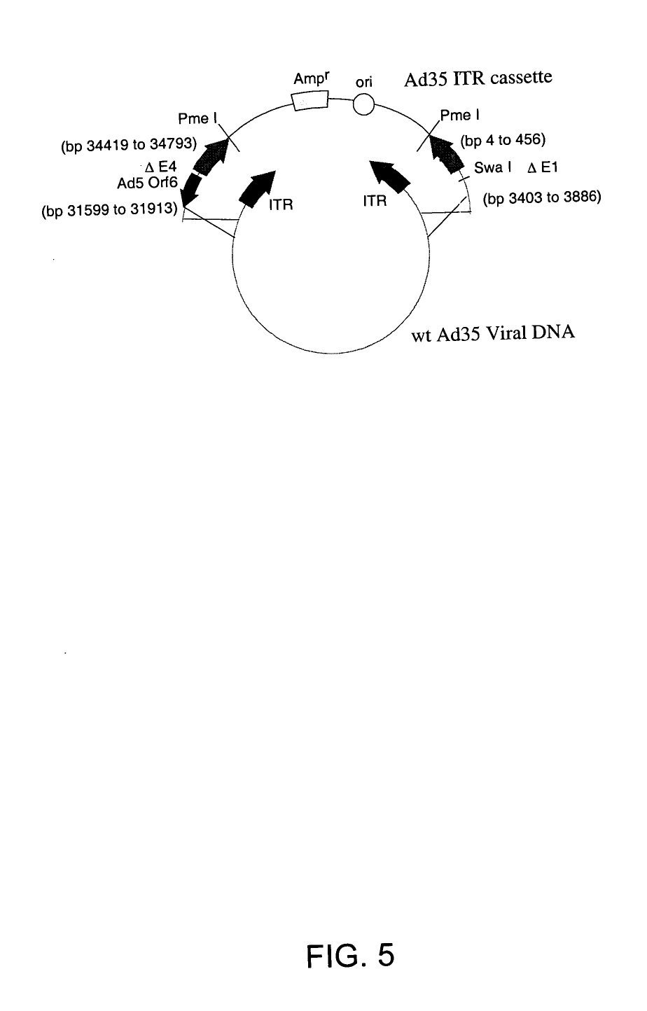

To construct pAd35ΔE1ΔE4Ad5Orf6 (An Ad35 pre-Ad plasmid containing an E1 deletion and an E4 deletion substituted with Ad5 Orf6), an Ad35 ITR cassette was constructed containing sequences from the right (bp 31599 to 31913 and bp 34419 to 34793) and left (bp 4 to 456 and bp 3403 to 3886) end of the Ad35 genome separated by plasmid sequences containing a bacterial origin of replication and an ampicillin resistance gene. These four segments were generated by PCR and cloned sequentially into pNEB193, generating pNEBAd35-4. Next the Ad5 Orf6 open reading frame was generated by PCR and cloned between Ad35 bp 31913 and 34419 generating pNEBAd35-4Ad5Orf6 (the ITR cassette). PNEB193 is a commonly used commercially available cloning plasmid (New England Biolabs cat# N3051 S) containing a bacterial origin of replication, ampicillin resistance gene and a multiple cloning site into which the PCR products were introduced. The ITR cassette contains a deletion of E1 sequences from Ad35 bp 457 to 3402 with a unique Swa I restriction site located in the deletion and an E4 deletion from Ad35 bp 31912 to 34418 into which Ad5 Orf6 was introduced in an E4 parallel orientation. In this construct, Ad5Orf6 expression is driven by the Ad35 E4 promoter. The Ad35 sequences (bp 31599 to 31913 and bp 3403 to 3886) in the ITR cassette provide regions of homology with the purified Ad35 viral DNA in which bacterial recombination can occur following cotransformation into BJ 5183 bacteria (FIG. 5). The ITR cassette was also designed to contain unique restriction enzyme sites (PmeI) located at the end of the viral ITR's so that digestion will release the recombinant Ad35 genome from plasmid sequences. Potential clones were screened by restriction analysis and one clone was selected as pAd35ΔE1ΔE4Ad5Orf6. Pre-Adenovirus plasmid pAd35ΔE1ΔE4Ad5Orf6 contains Ad35 sequences from bp 4 to 456; bp 3403 to bp 31913 and bp 34419 to bp 34793 with Ad5Orf6 cloned between bp 31913 and bp 34419.

To construct pAd35ΔE1ΔOrf6Ad5Orf6 (An Ad35 pre-Ad plasmid containing an E1 deletion and a deletion of E4 Orf6 substituted with Ad5 Orf6), an Ad35 ITR cassette was constructed containing sequences from the right (bp 31599 to 32081 and bp 32990 to 34793) and left (bp 4 to 456 and bp 3403 to 3886) end of the Ad35 genome separated by plasmid sequences containing a bacterial origin of replication and an ampicillin resistance gene. These four segments were generated by PCR and cloned sequentially into pNEB193, generating pNEBAd35-10. Next the Ad5 Orf6 open reading frame was generated by PCR and cloned between Ad35 bp 32081 and 32990 generating pNEBAd35-10Ad5Orf6 (the ITR cassette). PNEB193 is a commonly used commercially available cloning plasmid (New England Biolabs cat# N3051S) containing a bacterial origin of replication, ampicillin resistance gene and a multiple cloning site into which the PCR products were introduced. The ITR cassette contains a deletion of E1 sequences from Ad35 bp 457 to 3402 with a unique Swa I restriction site located in the deletion and a deletion of E4 Orf6 from Ad35 bp 32082 to 32989 into which Ad5 Orf6 was introduced in an E4 parallel orientation. In this construct, Ad5Orf6 expression is driven by the Ad35 E4 promoter. The Ad35 sequences (bp 31599 to 32081 and bp 3403 to 3886) in the ITR cassette provide regions of homology with the purified Ad35 viral DNA in which bacterial recombination can occur following cotransformation into BJ 5183 bacteria. The ITR cassette was also designed to contain unique restriction enzyme sites (Pme I) located at the end of the viral ITR's so that digestion will release the recombinant Ad35 genome from plasmid sequences. Potential clones were screened by restriction analysis and one clone was selected as pAd35ΔE1ΔOrf6Ad5Orf6. Pre-Adenovirus plasmid pAd35ΔE1ΔOrf6Ad5Orf6 contains Ad35 sequences from bp 4 to 456; bp 3403 to bp 32081 and bp 32990 to bp 34793 with Ad5Orf6 cloned between bp 32081 and bp 32990.

To construct pAd35ΔE1ΔE4Ad5E4 (An Ad35 pre-Ad plasmid containing an E1 deletion and a deletion of E4 substituted with Ad5 E4), an Ad35 ITR cassette was constructed containing sequences from the right (bp 31599 to 31838 and bp 34419 to 34793) and left (bp 4 to 456 and bp 3403 to 3886) end of the Ad35 genome separated by plasmid sequences containing a bacterial origin of replication and an ampicillin resistance gene. These four segments were generated by PCR and cloned sequentially into pNEB193, generating pNEBAd35-7. Next the Ad5 E4 coding region was generated by PCR and cloned between Ad35 bp 31838 and 34419 generating pNEBAd35-7Ad5E4-2 (the ITR cassette). PNEB193 is a commonly used commercially available cloning plasmid (New England Biolabs cat# N3051 S) containing a bacterial origin of replication, ampicillin resistance gene and a multiple cloning site into which the PCR products were introduced. The ITR cassette contains a deletion of E1 sequences from Ad35 bp 457 to 3402 with a unique Swa I restriction site located in the deletion and an E4 deletion from Ad35 bp 31839 to 34418 into which the Ad5 E4 coding region was introduced in an E4 parallel orientation. In this construct, the Ad5 E4 region is expressed using the Ad35 E4 promoter. The Ad35 sequences (bp 31599 to 31838 and bp 3403 to 3886) in the ITR cassette provide regions of homology with the purified Ad35 viral DNA in which bacterial recombination can occur following cotransformation into BJ 5183 bacteria. The ITR cassette was also designed to contain unique restriction enzyme sites (Pme I) located at the end of the viral ITR's so that digestion will release the recombinant Ad35 genome from plasmid sequences. Potential clones were screened by restriction analysis and one clone was selected as pAd35ΔE1ΔE4Ad5E4. Pre-Adenovirus plasmid pAd35ΔE1ΔE4Ad5E4 contains Ad35 sequences from bp 4 to 456; bp 3403 to bp 31838 and bp 34419 to bp 34793 with the Ad5 E4 coding region (Ad 5 bp 32914 to bp 35523) cloned between bp 31838 and bp 34419.

To construct pAd35ΔE1ΔE4Ad5PE4 (An Ad35 pre-Ad plasmid containing an E1 deletion and a deletion of E4 coding region and promoter substituted with Ad5 E4 coding region and promoter), an Ad35 ITR cassette was constructed containing sequences from the right (bp 31599 to 31838 and bp 34660 to 34793) and left (bp 4 to 456 and bp 3403 to 3886) end of the Ad35 genome separated by plasmid sequences containing a bacterial origin of replication and an ampicillin resistance gene. These four segments were generated by PCR and cloned sequentially into pNEB193, generating pNEBAd35-8. Next the Ad5 E4 promoter and coding region was generated by PCR and cloned between Ad35 bp 31838 and 34660 generating pNEBAd35-8Ad5E4PC (the ITR cassette). PNEB193 is a commonly used commercially available cloning plasmid (New England Biolabs cat# N3051S) containing a bacterial origin of replication, ampicillin resistance gene, and a multiple cloning site into which the PCR products were introduced. The ITR cassette contains a deletion of E1 sequences from Ad35 bp 457 to 3402 with a unique Swa I restriction site located in the deletion and an E4 deletion from Ad35 bp 31839 to 34659 into which the Ad5 E4 promoter and coding region was introduced in an E4 parallel orientation. In this construct, the Ad5 E4 region is expressed using the Ad5 E4 promoter. The Ad35 sequences (bp 31599 to 31838 and bp 3403 to 3886) in the ITR cassette provide regions of homology with the purified Ad35 viral DNA in which bacterial recombination can occur following cotransformation into BJ 5183 bacteria. The ITR cassette was also designed to contain unique restriction enzyme sites (Pme I) located at the end of the viral ITR's so that digestion will release the recombinant Ad35 genome from plasmid sequences. Potential clones were screened by restriction analysis and one clone was selected as pAd35ΔE1ΔE4Ad5PE4. Pre-Adenovirus plasmid pAd35ΔE1ΔE4Ad5PE4 contains Ad35 sequences from bp 4 to 456; bp 3403 to bp 31838 and bp 34660 to bp 34793 with the Ad5 E4 promoter and coding region (Ad 5 bp 32914 to bp 35826) cloned between bp 31838 and bp 34660.

### EXAMPLE 3

Rescue of pAd35ΔE1ΔE4Ad5Orf6, pAd35ΔE1ΔOrf6Ad5Orf6, pAd35ΔE1ΔE4Ad5E4 and pAd35ΔE1ΔE4Ad5PE4 into Virus

In order to determine if pre-adenovirus plasmids pAd35ΔE1ΔE4Ad5Orf6, pAd35ΔE1ΔOrf6Ad5Orf6, pAd35ΔE1ΔE4Ad5E4 and pAd35ΔE1ΔE4Ad5PE4 could be rescued into virus and propagated in a group C E1 complementing cell line, the plasmids were each digested with Pme I and transfected into T-25 flasks of PER.C6 cells using the calcium phosphate co-precipitation technique; Cell Phect Transfection Kit, Amersham Pharmacia Biotech Inc. PmeI digestion releases the viral genome from plasmid sequences allowing viral replication to occur after cell entry. Viral cytopathic effect (CPE), indicating that virus replication and amplification was occurring, was observed for all construct. When CPE was complete, approximately 7-10 days post transfection, the infected cells and media were harvested, freeze/thawed three times and the cell debris pelleted by centrifugation. Approximately 1 ml of the cell lysate was used to infect aT-225 flasks of PER.C6 cells at 80-90% confluence. Once CPE was reached, infected cells and media were harvested, freeze/thawed three times and the cell debris pelleted by centrifugation. Clarified cell lysates were then used to infect 2-layer NUNC cell factories of PER.C6 cells. Following complete CPE the virus was purified by ultracentrifugation on CsCl density gradients. In order to verify the genetic structure of the rescued viruses, viral DNA was extracted using pronase treatment followed by phenol chloroform extraction and ethanol precipitation. Viral DNA was then digested with HindIII and treated with Klenow fragment to end-label the restriction fragments with P33-dATP. The end-labeled restriction fragments were then size-fractionated by gel electrophoresis and visualized by autoradiography. The digestion products were compared with the digestion products of the corresponding pre-Adenovirus plasmid (that had been digested with Pme1/HindIII prior to labeling) from which they were derived. The expected sizes were observed, indicating that the viruses had been successfully rescued.

### EXAMPLE 4

Insertion of an Expression Cassette into pAd35ΔE1ΔE4Ad5Orf6, pAd35ΔE1ΔOrf6Ad5Orf6, pAd35ΔE1ΔE4Ad5E4 and pAd35ΔE1ΔE4Ad5PE4

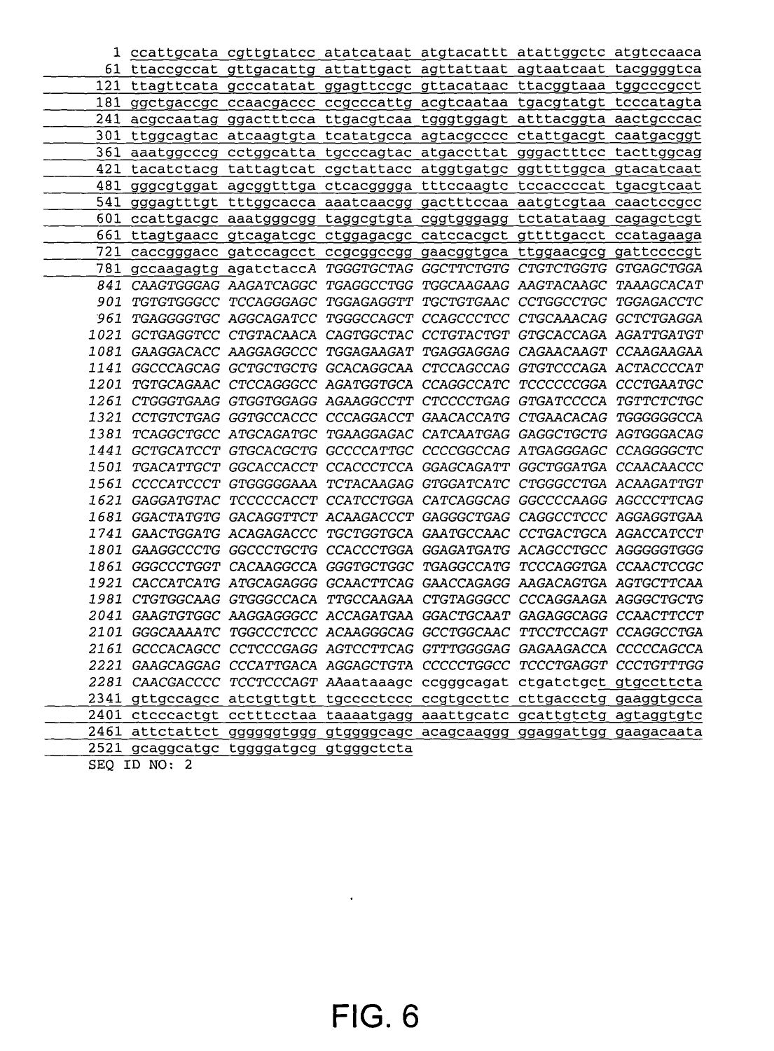

In order to introduce a gag or SEAP expression cassette into the E1 region of the various Ad35 pre-Adenovirus plasmids described above (pAd35ΔE1ΔE4Ad5Orf6, pAd35ΔE1ΔOrf6Ad5Orf6, pAd35ΔE1ΔE4Ad5E4 and pAd35ΔE1ΔE4Ad5PE4) bacterial recombination was again used. A gag expression cassette consisting of the following: 1) the immediate early gene promoter from the human cytomegalovirus, 2) the coding sequence of the human immunodeficiency virus type 1 (HIV-1) gag (strain CAM-1; 1526 bp) gene, and 3) the bovine growth hormone polyadenylation signal sequence (FIG. 6), was cloned into the E1 deletion in Ad35 shuttle plasmid, pNEBAd35-2 (a precursor to the Ad35 ITR cassettes described above), generating pNEBAd35CMVgagBGHpA. pNEBAd35-2 contains Ad35 sequences from the left end of the genome (bp 4 to 456 and bp 3403 to 3886) with a unique SwaI site between bp 456 and 3403 at the position of the deletion. The gag expression cassette was obtained from a previously constructed shuttle plasmid by EcoRI digestion. Following the digestion the desired fragment was gel purified, treated with Klenow to obtain blunt ends and cloned into the SwaI site in pNEBAd35-2. This cloning step resulted in the gag expression cassette being cloned into the E1 deletion between bp 456 and 3403 in the E1 parallel orientation. The shuttle vector containing the gag transgene was digested to generate a DNA fragment consisting of the gag expression cassette flanked by Ad35 bp 4 to 456 and bp 3403 to 3886 and the fragment was purified after electrophoresis on an agarose gel. Cotransformation of BJ 5183 bacteria with the shuttle vector fragment and one of the Ad35 pre-Ad plasmids (pAd35ΔE1ΔE4Ad5Orf6, pAd35ΔE1ΔOrf6Ad5Orf6, pAd35ΔE1ΔE4Ad5E4, pAd35ΔE1ΔE4Ad5PE4), linearized in the E1 region by digestion with Swa I, resulted in the generation of corresponding Ad35 gag-containing pre-Adenovirus plasmids (pAd35ΔE1gagΔE4Ad5Orf6, pAd35ΔE1 gagΔOrf6Ad5Orf6, pAd35ΔE1gagΔE4Ad5E4, and pAd35ΔE1gagΔE4Ad5PE4) by homologous recombination. Potential clones were screened by restriction analysis.

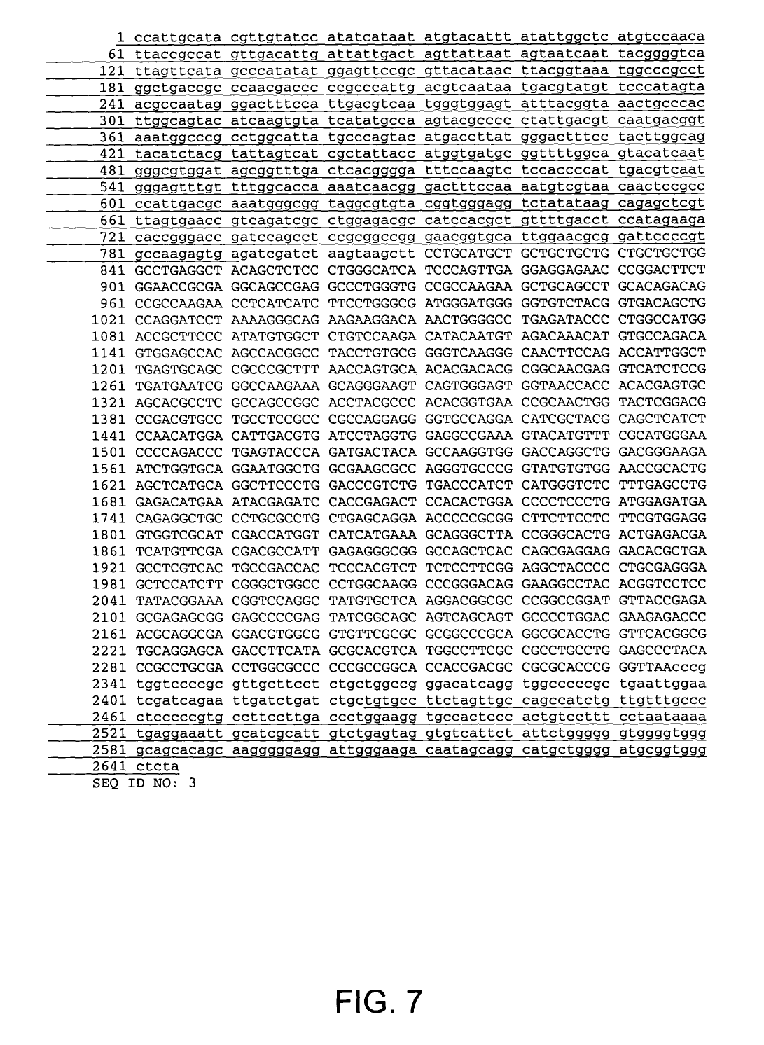

A similar strategy was used to generate Ad35 pre-Ad plasmids containing a SEAP expression cassette. In this case a SEAP expression cassette consisting of: 1) the immediate early gene promoter from the human cytomegalovirus, 2) the coding sequence of the human placental SEAP gene, and 3) the bovine growth hormone polyadenylation signal sequence (FIG. 7) was cloned into the E1 deletion in Ad35 shuttle plasmid, pNEBAd35-2, generating pNEBAd35CMVSEAPBGHpA. The SEAP expression cassette was obtained from a previously constructed shuttle plasmid by EcoRI digestion. Following the digestion the desired fragment was gel purified, treated with Klenow to obtain blunt ends and cloned into the SwaI site in pNEBAd35-2. The transgene was then recombined into the various Ad35 backbones generating pAd35ΔE1SEAPΔE4Ad5Orf6, pAd35ΔE1SEAPΔOrf6Ad5Orf6, pAd35ΔE1SEAPΔE4Ad5E4, and pAd35ΔE1SEAPΔE4Ad5PE4 as described above for the gag transgene. All pre-Ad plasmids were rescued into virus and expanded to prepare CsCl purified stocks as described above.

### EXAMPLE 5

In vivo Transgene Expression

A. Immunization

Female mice were between 4-10 weeks old. The total dose of each vaccine was suspended in 0.1 mL of buffer. The vectors were given to both quadriceps of each animals with a volume of 50 μL per quad and using 0.3-mL 28G1/2 insulin syringes (Becton-Dickinson, Franklin Lakes, N.J.). The rhesus macaques and African green monkeys were between 2-5 kg in weight. For the primates, the total dose of each vaccine was suspended in 1 mL of buffer. The monkeys were anesthetized (ketamine/xylazine mixture) and the vaccines were delivered i.m. in 0.5-mL aliquots into two muscle sites using tuberculin syringes (Becton-Dickinson, Franklin Lakes, N.J.). Serum samples were collected at defined intervals and stored frozen until the assay date. All animal care and treatment were in accordance with standards approved by the Institutional Animal Care and Use Committee according to the principles set forth in the Guide for Care and Use of Laboratory Animals, Institute of Laboratory Animal Resources, National Research Council.

B. SEAP Assay

Serum samples were analyzed for circulating SEAP levels using TROPIX phospha-light chemiluminescent kit (Applied Biosystems Inc). Duplicate 5 μL aliquots of each serum were mixed with 45 μL of kit-supplied dilution buffer in a 96-well white DYNEX plate. Serially diluted solutions of a human placental alkaline phosphatase (Catalog no. M5905, Sigma, St. Louis, Mo.) in 10% naïve monkey or mouse serum served to provide the standard curve. Endogenous SEAP activity in the samples was inactivated by heating the well for 30 minutes at 65° C. Enzymatic SEAP activities in the samples were determined following the procedures described in the kit. Chemiluminescence readings (in relative light units) were recorder using DYNEX luminometer. RLU readings are converted to ng/mL SEAP using a log-log regression analyses.

C. Rodent Results

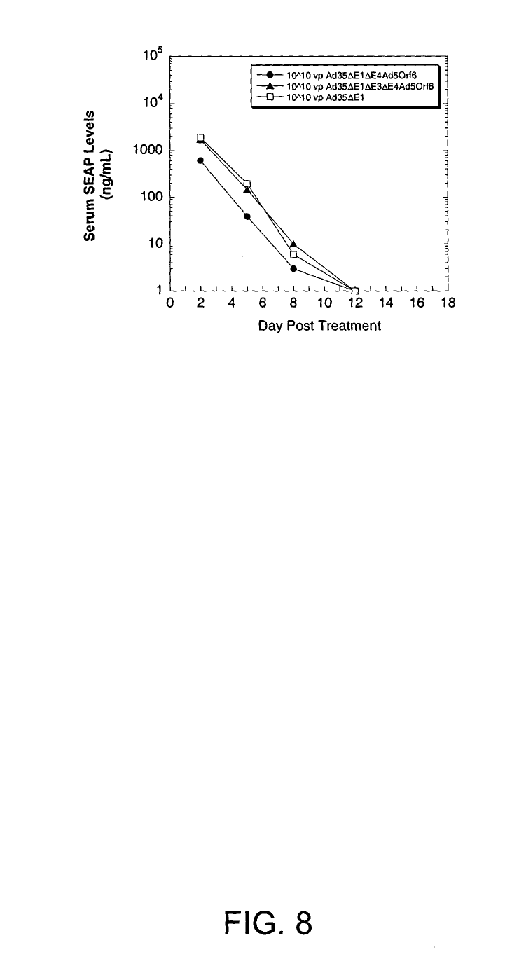

In the first mouse experiment, cohorts of 5 C3H/HeN mice were given single intramuscular injections of one of the following vectors: (1) 10\hat{ }10 vp Ad35ΔE1SEAPΔE4Ad5Orf6; (2) 10\hat{ }10 vp Ad35ΔE1SEAPΔE3ΔE4Ad5Orf6; or (3) 10\hat{ }10 vp Ad35ΔE1SEAP. Serum samples prior to and after the injection were analyzed for circulating SEAP activities and the results are shown in FIG. 8. Results indicate that (1) the Ad35 constructs are all capable of expressing the SEAP transgene and that (2) the introduction of Ad5Orf6 sequence where the deleted Ad35E4 was did not significantly affect the transgene expression relative to Ad35ΔE1SEAP. Ad35ΔE1SEAPΔE3ΔE4Ad5Orf6 also yielded a similar expression profile as Ad35ΔE1SEAP. The levels of SEAP in the serum dropped after day 2 and were at background levels by day 12.

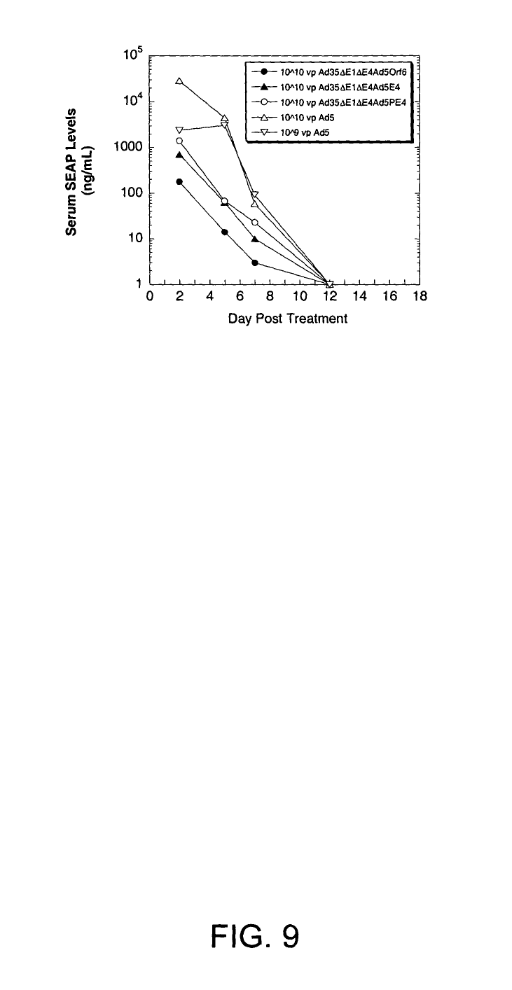

The second mouse experiment evaluates the effect of a full Ad5E4 replacement instead of an Ad5Orf6 substitution for the Ad35 E4 cassette. Here, cohorts of 5 C3H/HeN mice were given single intramuscular injections of one of the following vectors: (1) 10\hat{ }10 vp MRKAd5-SEAP; (2) 10\hat{ }9 vp MRKAd5-SEAP; (3) 10\hat{ }10 vp Ad35ΔE1SEAPΔE4Ad5Orf6; (4) 10\hat{ }10 vp Ad35ΔE1SEAPΔE4Ad5E4; or (5) 10\hat{ }10 vp Ad35ΔE1SEAPΔE4Ad5PE4. The introduction of Ad5E4 or Ad5PE4 resulted in comparable if not, slightly improved expression levels compared to the vector with the Ad5Orf6 insertion (FIG. 9). The peak levels for the Ad35 constructs are lower than those produced by Ad5SEAP (at least 10-fold).

D. Primate Results

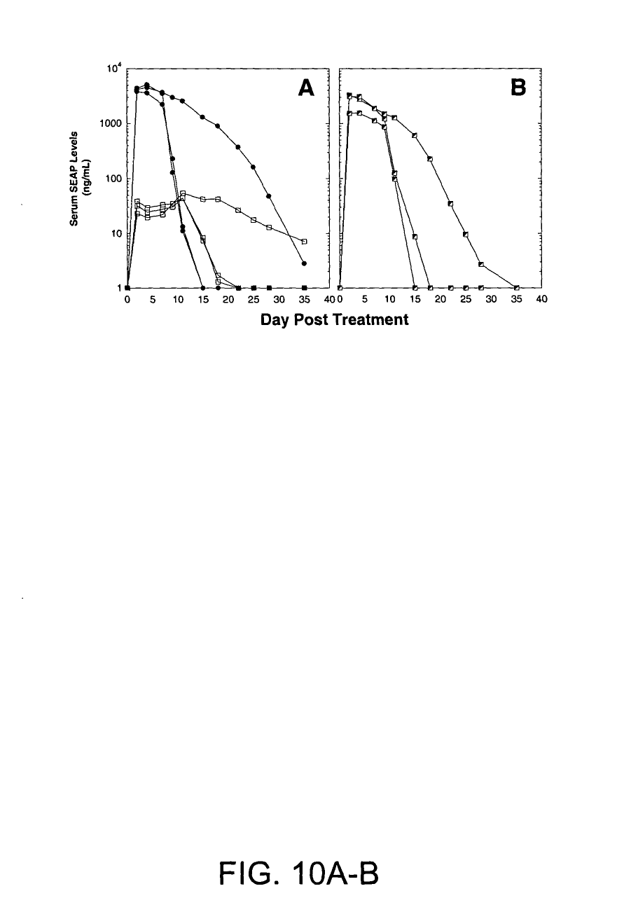

Cohorts of 3 rhesus macaques were given single intramuscular injections of one of the following vectors: (1) 10\hat{ }11 vp MRKAd5-SEAP; (2) 10\hat{ }9 vp MRKAd5-SEAP; or (3) 10\hat{ }11 vp Ad35ΔE1SEAPΔE4Ad5Orf6. Serum samples prior to and after the injection were analyzed for circulating SEAP activities and the results for the individual monkeys are shown in FIGS. 10A-B. Results indicate that the peak level of SEAP product produced by the alternative adenovirus serotype was lower than but were within 3-fold of that of MRKAd5SEAP at the same high dose level of 10\hat{ }11 vp. The levels observed from the Ad35 vector were about 50-fold higher than those observed using 10\hat{ }9 vp of MRKAd5SEAP. The levels of SEAP in the serum dropped after day 10 and were close to background as early as day 15.

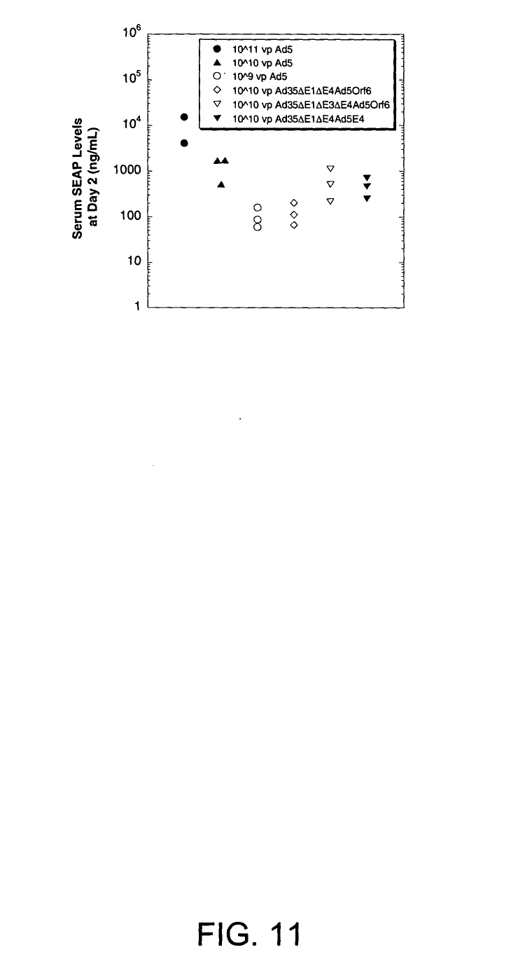

A separate experiment using African green monkeys was conducted to examine the effect of the additional E3 deletion or the full Ad5E4 substitution on in vivo gene expression. In here, cohorts of 2-3 African green macaques were given single intramuscular injections of one of the following vectors: (1) 10\hat{ }11 vp MRKAd5-SEAP; (2) 10\hat{ }10 vp MRKAd5-SEAP; (3) 10\hat{ }9 vp MRKAd5-SEAP; (4) 10\hat{ }10 vp Ad35ΔE1SEAPΔE4Ad5Orf6; (5) 10\hat{ }10 vp Ad35ΔE1SEAPΔE3ΔE4Ad5Orf6; or (6) 10\hat{ }10 vp Ad35ΔE1SEAPΔE4Ad5E4. Results (FIG. 11) indicate that the peak levels of SEAP product produced by Ad35ΔE1SEAPΔE3ΔE4Ad5Orf6 and Ad35ΔE1SEAPΔE4Ad5E4 were comparable if not, slightly improved compared to Ad35ΔE1SEAPΔE4Ad5Orf6.

### EXAMPLE 6

In Vivo Immunogenicity

A. Immunization

Cohorts of 3-6 animals were given intramuscular injections at wk 0 and wk 4 of either of the following constructs: (1) 10\hat{ }11 vp MRKAd5-HIV1 gag; or (2) 10\hat{ }11 vp of Ad35ΔE1gagΔE4Ad5Orf6. Rhesus macaques were between 3-10 kg in weight. In all cases, the total dose of each vaccine was suspended in 1 mL of buffer. The macaques were anesthetized (ketamine/xylazine) and the vaccines were delivered i.m. in 0.5-mL aliquots into both deltoid muscles using tuberculin syringes (Becton-Dickinson). Sera and peripheral blood mononuclear cells (PBMC) were prepared from blood samples collected at several time points during the immunization regimen. All animal care and treatment were in accordance with standards approved by the Institutional Animal Care and Use Committee according to the principles set forth in the Guide for Care and Use of Laboratory Animals, Institute of Laboratory Animal Resources, National Research Council.

B. ELISPOT Assay

The IFN-γ ELISPOT assays for rhesus macaques were conducted following a previously described protocol (Allen et al., 2001 J. Virol. 75(2):738-749), with some modifications. For antigen-specific stimulation, a peptide pool was prepared from 20-aa peptides that encompass the entire HIV-1 gag sequence with 10-aa overlaps (Synpep Corp., Dublin, Calif.). To each well, 50 μL of 2-4×10⁵ peripheral blood mononuclear cells (PBMCs) were added; the cells were counted using Beckman Coulter Z2 particle analyzer with a lower size cut-off set at 80 femtoliters (“fL”). Either 50 μL of media or the gag peptide pool at 8 μg/mL concentration per peptide was added to the PBMC. The samples were incubated at 37° C., 5% CO₂ for 20-24 hrs. Spots were developed accordingly and the plates were processed using custom-built imager and automatic counting subroutine based on the ImagePro platform (Silver Spring, Md.); the counts were normalized to 10⁶ cell input.

C. Intracellular Cytokine Staining

To 1 ml of 2×10⁶ PBMC/mL in complete RPMI media (in 17×100 mm round bottom polypropylene tubes (Sarstedt, Newton, N.C.)), anti-hCD28 (clone L293, Becton-Dickinson) and anti-hCD49d (clone L25, Becton-Dickinson) monoclonal antibodies were added to a final concentration of 1 μg/mL. For gag-specific stimulation, 10 μL of the peptide pool (at 0.4 mg/mL per peptide) were added. The tubes were incubated at 37° C. for 1 hr., after which 20 μL of 5 mg/mL of brefeldin A (Sigma) were added. The cells were incubated for 16 hr at 37° C., 5% CO₂, 90% humidity. 4 mL cold PBS/2% FBS were added to each tube and the cells were pelleted for 10 min at 1200 rpm. The cells were re-suspended in PBS/2% FBS and stained (30 min, 4° C.) for surface markers using several fluorescent-tagged mAbs: 20 μL per tube anti-hCD3-APC, clone FN-18 (Biosource); 20 μL anti-hCD8-PerCP, clone SK1 (Becton Dickinson, Franklin Lakes, N.J.); and 20 μL anti-hCD4-PE, clone SK3 (Becton Dickinson). Sample handling from this stage was conducted in the dark. The cells were washed and incubated in 750 μL 1×FACS Perm buffer (Becton Dickinson) for 10 min at room temperature. The cells were pelleted and re-suspended in PBS/2% FBS and 0.1 μg of FITC-anti-hIFN-γ, clone MD-1 (Biosource) was added. After 30 min incubation, the cells were washed and re-suspended in PBS. Samples were analyzed using all four color channels of the Becton Dickinson FACSCalibur instrument. To analyze the data, the low side- and forward-scatter lymphocyte population was initially gated; a common fluorescence cut-off for cytokine-positive events was used for both CD4⁺ and CD8⁺ populations, and for both mock and gag-peptide reaction tubes of a sample.

D. Results

PBMCs collected at regular 4-wk intervals were analyzed in an ELISPOT assay. Results (Table 1) indicate that the Ad35ΔE1gagΔE4Ad5Orf6 is able to induce in non-human primates significant levels of gag-specific T cells. After a single dose (wk 4), the Ad35-induced responses were about 5-fold lower than that of MRKAd5-HIV1 gag. After the second dose (wk 8), the responses between both cohorts were comparable; the differences became pronounced in the succeeding time points.

TABLE 1
Gag-specific T cell response in monkeys immunized with MRKAd5-HIV1gag and
Ad35ΔE1gagΔE4Ad5Orf6. Shown is the number of spot-forming cells per million PBMC
following incubation in the absence (mock) or presence of Gag H peptide pool. The H pool
consisted of 20-aa peptide overlapping by 10 aa and encompassing the entire gag sequence.

| | Vaccine | Monkey | Pre | | Wk 4 | | Wk 8 | | Wk 12 | | Wk 16 | |
|---|---|---|---|---|---|---|---|---|---|---|---|---|
| Grp | Wk 0, Wk 4 | ID | Mock | Gag H | Mock | Gag H | Mock | Gag H | Mock | Gag H | Mock | Gag H |
| 1 | MRKAd5-HIV1 gag | 00C018 | 1 | 5 | 13 | 1025 | 0 | 824 | 3 | 753 | 1 | 533 |
| | 10\hat{ }11 vp | 00C034 | 0 | 4 | 5 | 219 | 5 | 404 | 0 | 491 | 1 | 350 |
| | | 00C058 | 4 | 4 | 3 | 1086 | 0 | 440 | 0 | 439 | 0 | 599 |
| 2 | Ad35ΔE1gagΔE4Ad5Orf6 | 00D045 | 1 | 1 | 3 | 168 | 5 | 645 | 4 | 178 | 0 | 91 |
| | 10\hat{ }11 vp | 00D067 | 1 | 4 | 5 | 89 | 0 | 103 | 0 | 76 | 0 | 19 |
| | | 00D068 | 1 | 4 | 10 | 34 | 5 | 365 | 3 | 143 | 0 | 95 |
| | | 00D054 | 3 | 15 | 10 | 195 | 0 | 501 | 3 | 350 | 0 | 124 |
| | | 00D075 | 3 | 5 | 18 | 275 | 13 | 716 | 3 | 158 | 0 | 103 |
| | | 00D073 | 14 | 26 | 1 | 241 | 3 | 485 | 3 | 278 | 0 | 148 |
| 3 | Naïve | 00D087 | 1 | 1 | 3 | 3 | 8 | 54 | 3 | 5 | 3 | 1 |

Intracellular IFN-γ staining analyses of PBMC collected at wk 8 suggest that the Ad35-based vaccine is able to induce both HIV-specific CD4+ and CD8+ T cells (Table 2).

TABLE 2
Characterization of the gag-specific T cells in monkeys immunized
with MRKAd5-HIV1gag and Ad35ΔE1gagΔE4Ad5Orf6. Shown are the
percentages of CD3+ T cells that are either gag-specific CD4+
or gag-specific CD8+ cells. These values were corrected for
mock values (<0.02%).

| | | | Wk 8 | |
|---|---|---|---|---|
| | Vaccine | Monkey | % CD4 + | % CD8 + |
| Grp | Wk 0, Wk 4 | ID | CD3+ | CD3+ |
| 1 | MRKAd5-HIV1 gag | 00C018 | 0.08 | 0.37 |
| | 10\hat{ }11 vp | 00C034 | 0.09 | 0.06 |
| | | 00C058 | 0.03 | 0.21 |
| 2 | Ad35ΔE1gagΔE4Ad5Orf6 | 00D045 | 0.06 | 0.08 |
| | 10\hat{ }11 vp | 00D067 | 0.02 | 0.02 |
| | | 00D068 | 0.15 | 0.02 |
| | | 00D054 | 0.05 | 0.08 |
| | | 00D075 | 0.08 | 0.05 |
| | | 00D073 | 0.09 | 0.06 |

In a separate experiment, 3 different Ad35 constructs expressing HIV-1 gag were evaluated for their immunogenicity in macaques. Here, cohorts of 3 macaques were given immunizations at wk 0 and 4 of either of the following vectors: (1) 10\hat{ }10 vp Ad35ΔE1gagΔE4Ad5Orf6; (2) 10\hat{ }10 vp Ad35ΔE1gagΔE3ΔE4Ad5Orf6; or (3) 10\hat{ }10 vp Ad35ΔE1gagΔE4Ad5E4. The levels of T cell immunity induced by all 3 vectors were comparable at this stage (Table 2), suggesting that the additional E3 deletion or full Ad5E4 substitution does not appear to impair the immunogenic properties of the vector.

TABLE 3
Gag-specific T cell response in monkeys immunized with several Ad35ΔE1ΔE4-based
vectors. Shown is the number of spot-forming cells per million PBMC following
incubation in the absence (mocK0 or presence of Gag H peptide pool. The H pool consisted
of 20-aa peptide overlapping by 10 aa and encompassing the entire gag sequence.

| | Vaccine | Monkey | Pre | | Wk 4 | | Wk 8 | |
|---|---|---|---|---|---|---|---|---|
| Grp | Wk 0, Wk 4 | ID | Mock | Gag H | Mock | Gag H | Mock | Gag H |
| 1 | Ad35ΔE1gagΔE4Ad5Orf6 | 00C047 | 4 | 1 | 0 | 20 | 0 | 189 |
| | 10\hat{ }10vp | 00C157 | 8 | 5 | 1 | 81 | 1 | 833 |
| | | 00C078 | 3 | 1 | 0 | 46 | 4 | 349 |
| 2 | Ad35ΔE1gagΔE3ΔE4Ad5Orf6 | 00C091 | 1 | 1 | 1 | 118 | 3 | 315 |
| | 10\hat{ }10vp | 00C122 | 3 | 0 | 0 | 31 | 1 | 138 |
| | | 00D177 | 3 | 3 | 1 | 45 | 1 | 64 |
| 3 | Ad35ΔE1gagΔE4Ad5E4 | 00D018 | 3 | 19 | 29 | 120 | 23 | 193 |
| | 10\hat{ }10vp | 00D046 | 8 | 5 | 1 | 21 | 10 | 143 |
| | | 00D063 | 3 | 4 | 0 | 63 | 4 | 371 |
| Naïve | none | 00D363 | 0 | 5 | ND | ND | 0 | 0 |

### EXAMPLE 7

Construction and Rescue of pAd24ΔE1.

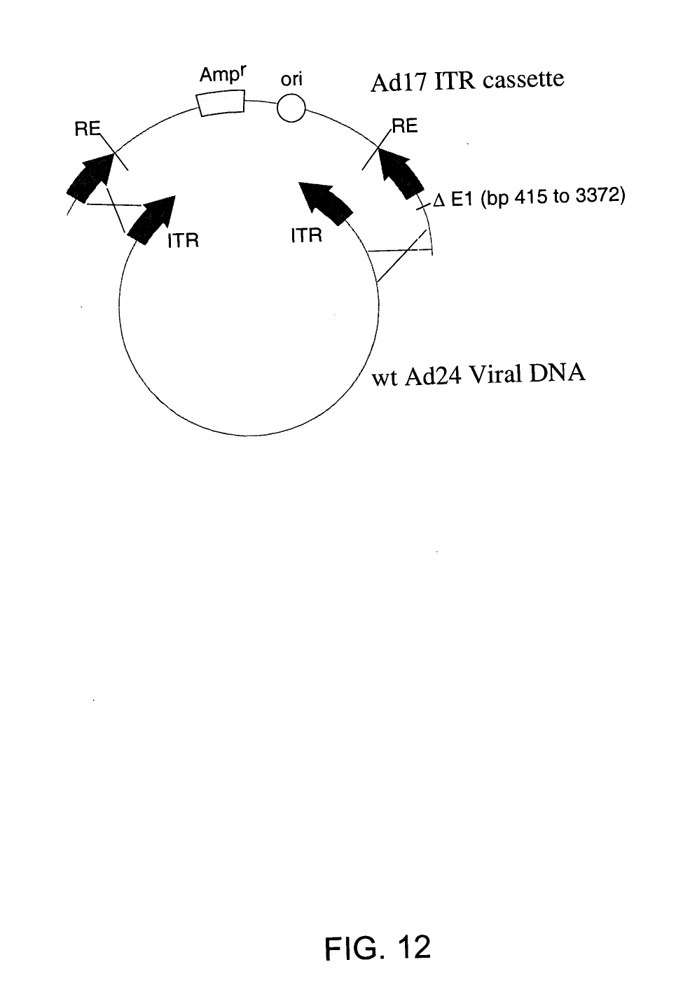

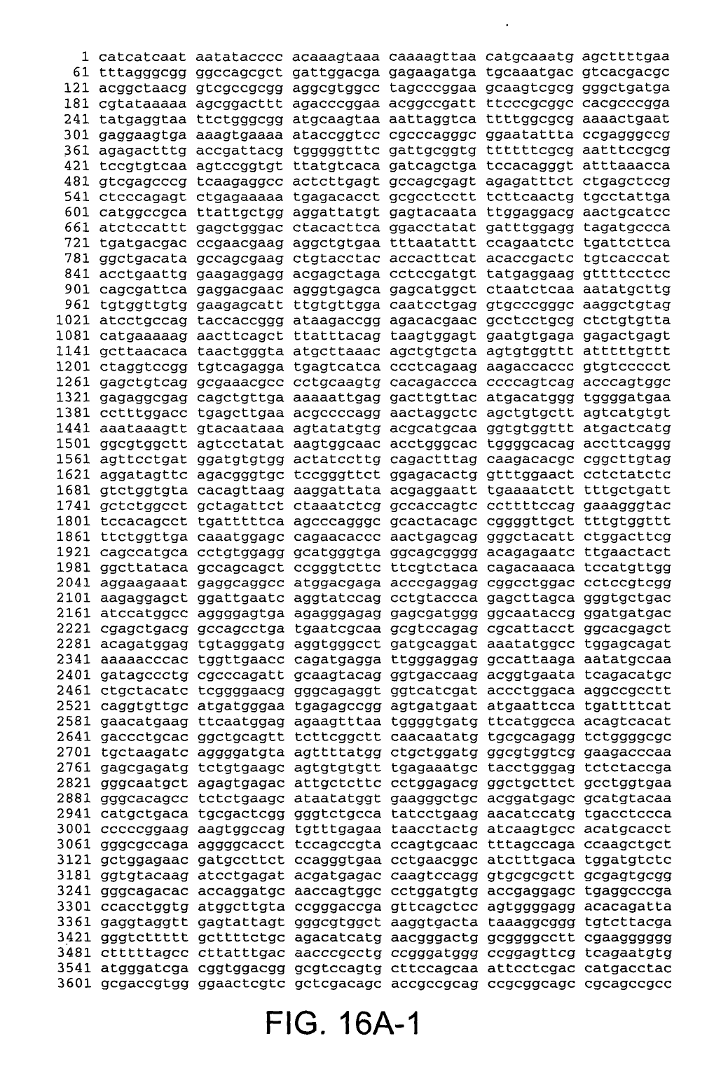

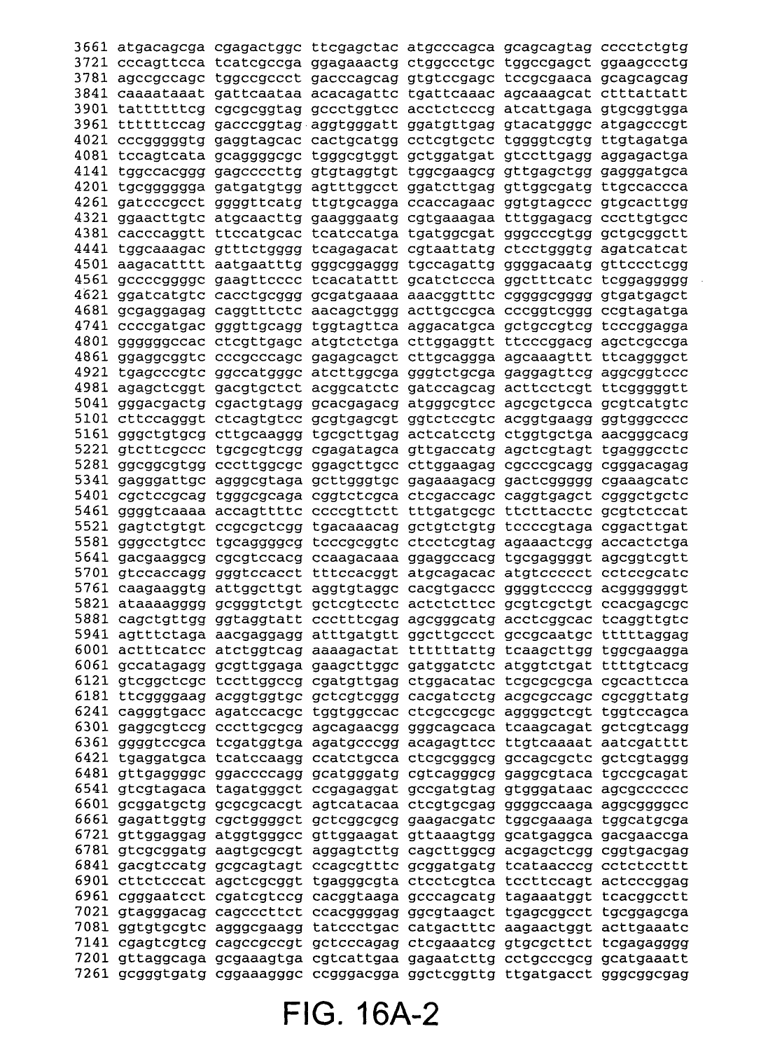

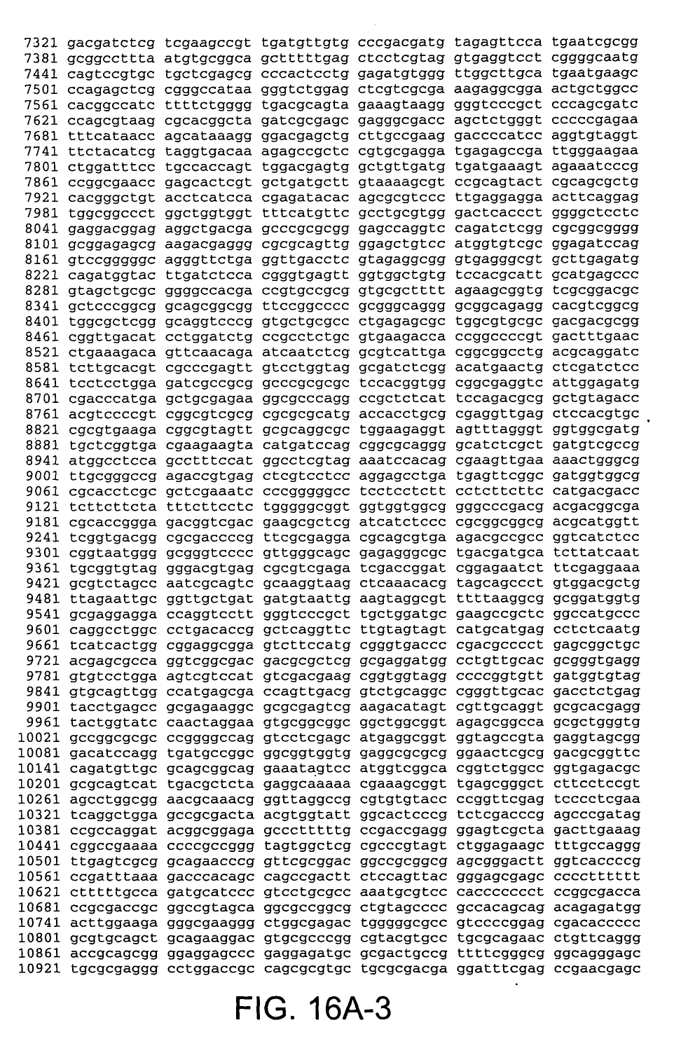

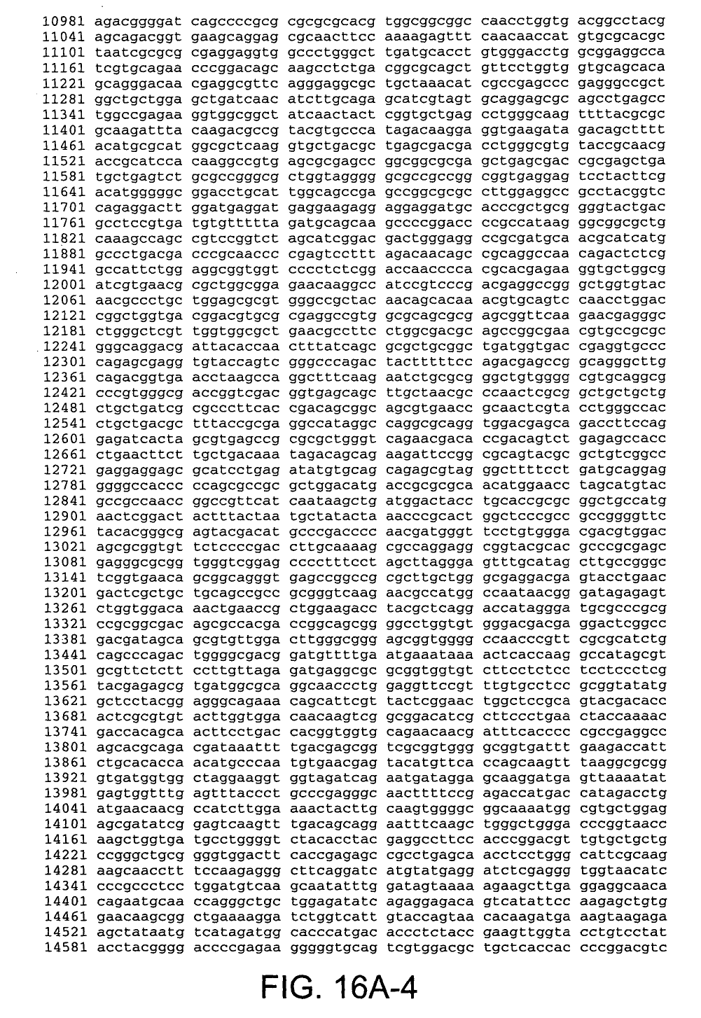

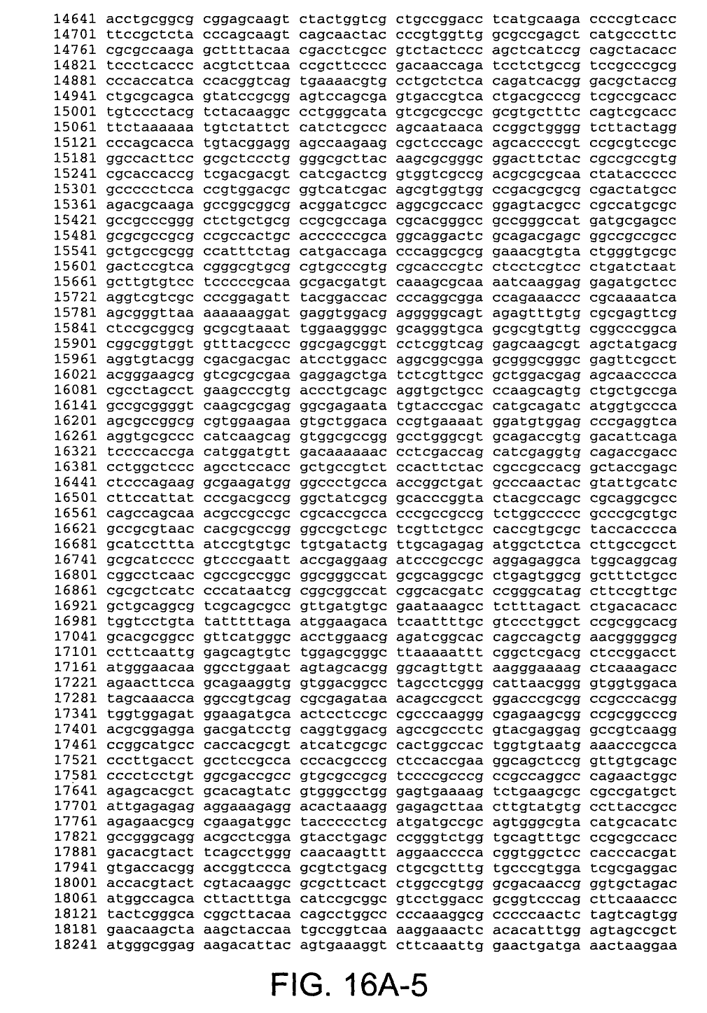

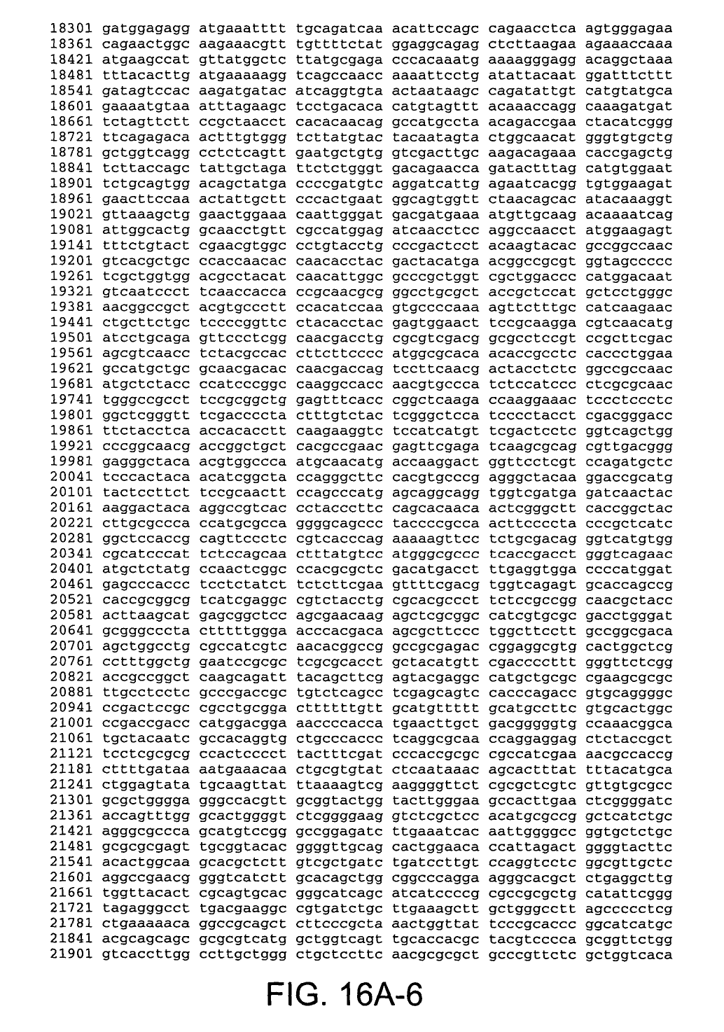

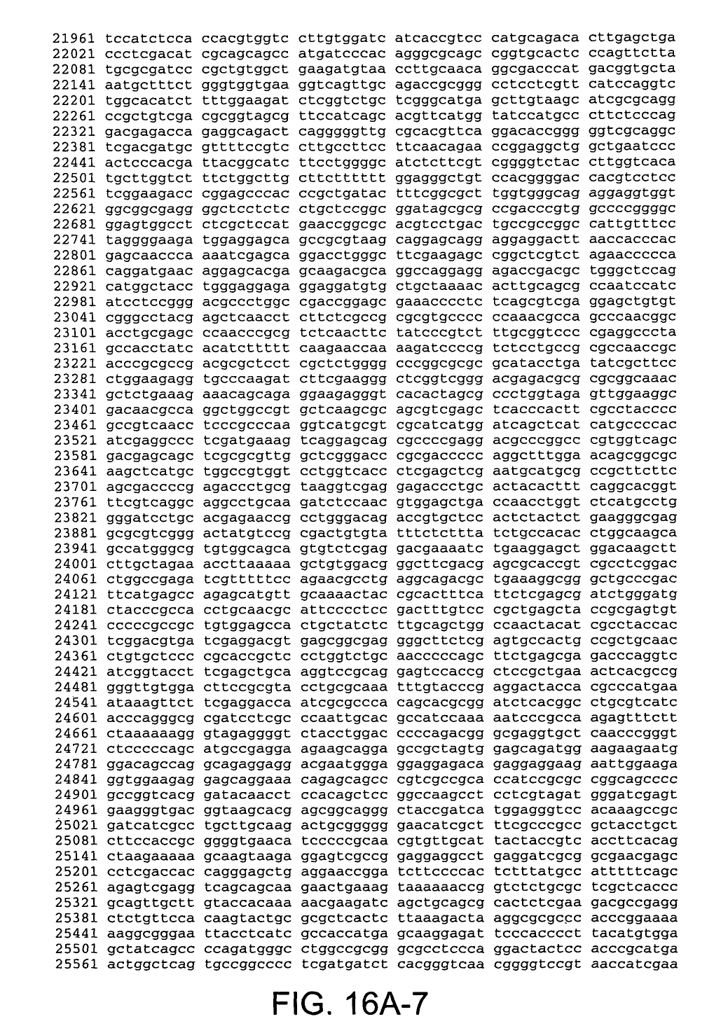

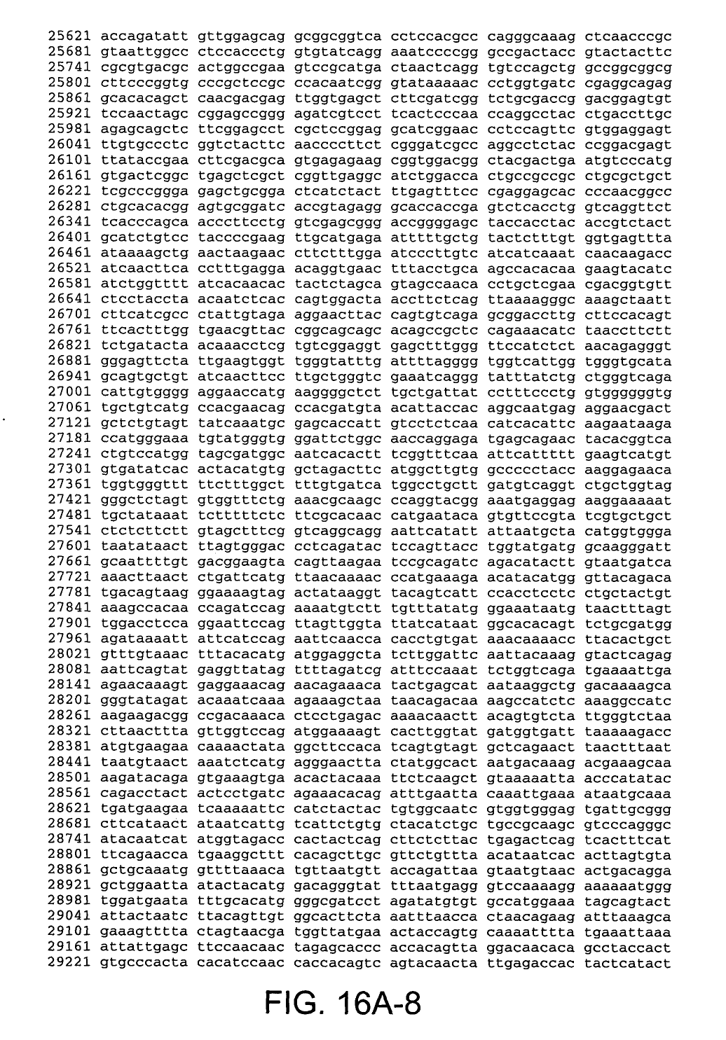

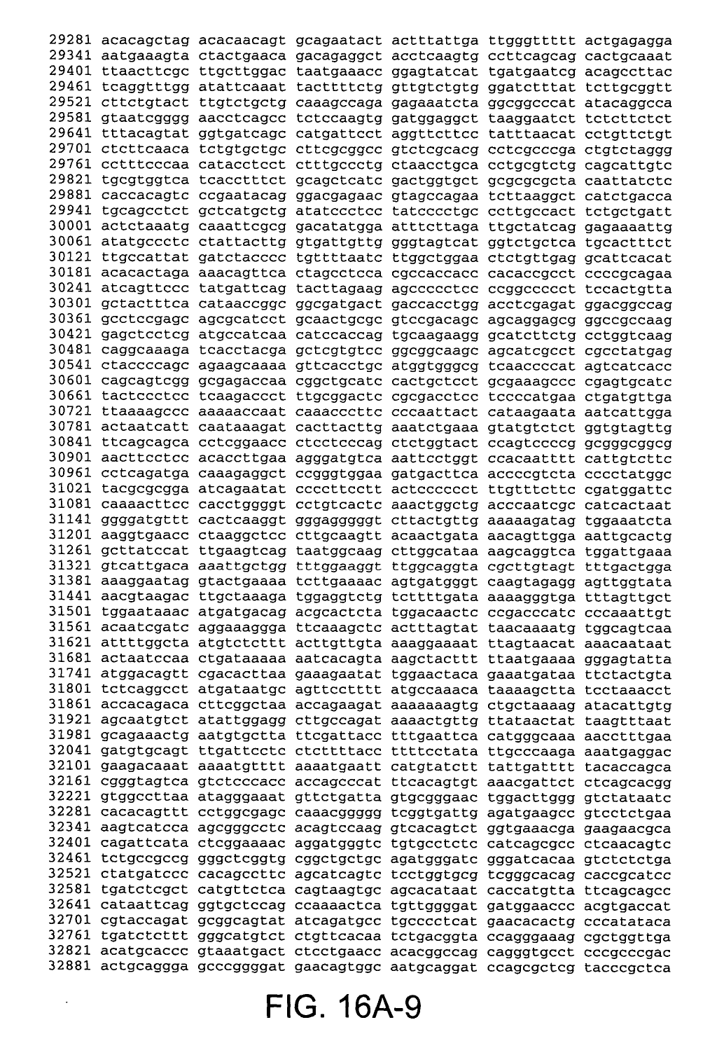

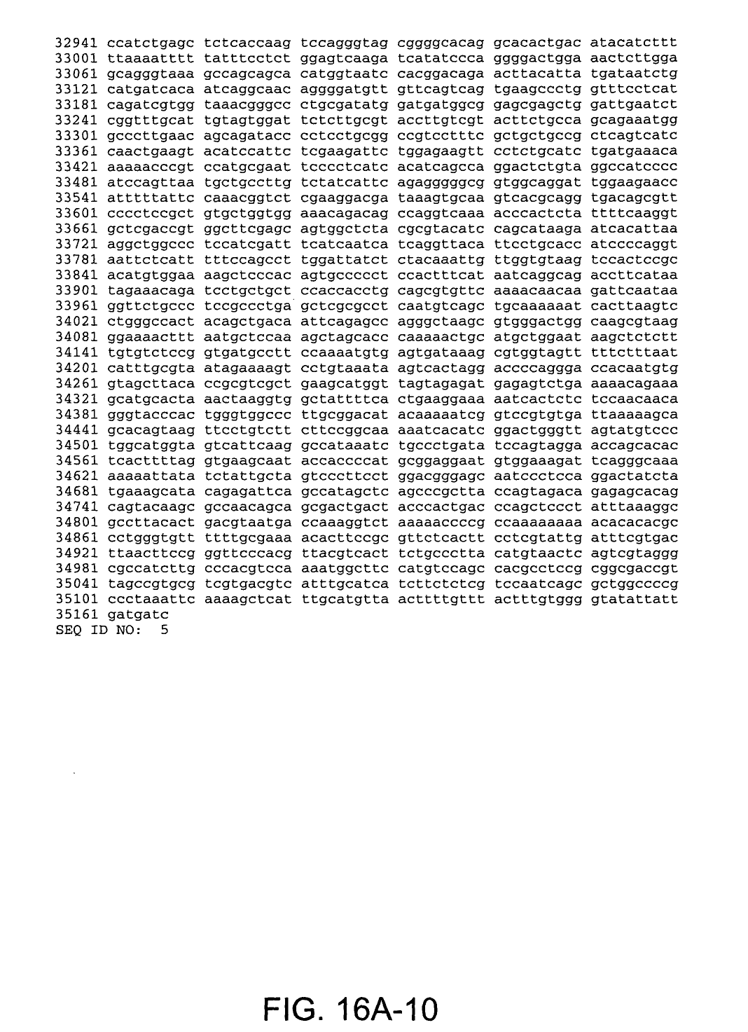

An E1-Ad24-based pre-adenovirus plasmid was constructed in order to determine whether an E1-Ad24 vector (a representative group D serotype) could be propagated in an Ad5/group C E1-complementing cell line. Since at the time the vector construction was initiated the complete sequence of Ad24 (see FIGS. 16A-1 through 16A-10; subject of copending application serial No. 60/455,312, filed Mar. 17, 2003) was unknown we took advantage of some sequence homology between Ad24 and Ad17. The general strategy used to recover Ad24 as a bacterial plasmid is illustrated in FIG. 12 and described below. Cotransformation of BJ5183 bacteria with purified wild-type Ad24 viral DNA and a second DNA fragment termed the Ad 17 ITR cassette resulted in the circularization of the viral genome by homologous recombination. The ITR cassette contains sequences from the right (bp 34469 to 35098) and left (bp 4 to 414 and bp 3373 to 4580) end of the Ad17 genome (Accession No. AF108105) separated by plasmid sequences containing a bacterial origin of replication and an Ampicillin resistance gene. The ITR cassette contains a deletion of E1 sequences from Ad17 (bp 415 to 3372) with a unique Swa I site located in the deletion. The Ad17 sequences in the ITR cassette provide regions of homology with the purified Ad24 viral DNA in which recombination can occur. The ITR cassette was also designed to contain unique restriction enzyme sites (Pme I) located at the end of the viral ITR's so that digestion will release the Ad24 genome from plasmid sequences. Potential clones were screened by restriction analysis and one clone was selected as pAd24ΔE1. pAd24ΔE1 contains Ad17 sequences from bp 4 to 414 and from bp 3373 to 4580, Ad24 bp 4588 to 34529, and Ad17 bp 34469 to 35098 (bp numbers refer to the wt sequence for both Ad17 and Ad24). PAd24ΔE1 contains the coding sequences for all Ad24 virion structural proteins that constitute its serotype specificity. This approach can be used to circularize any group D serotype into plasmid form which has sufficient homology to Ad17.

To determine if pre-adenovirus plasmid pAd24ΔE1 could be rescued into virus and propagated in a group C E1 complementing cell line, the plasmid was digested with Pme I and transfected into a 6 cm dish of 293 cells using the calcium phosphate co-precipitation technique. Pme I digestion releases the viral genome from the plasmid sequences allowing viral replication to occur after entry into 293 cells. Viral cytopathic effect (CPE), indicating that virus replication and amplification is occurring, was very slow to arise. Following multiple attempts, we were successful at rescuing and amplifying Ad24ΔE1 but the virus grew to lower titers and took more passages to amplify than a similar Ad5 based vector. In order to verify the genetic structure of the virus, viral DNA was extracted using pronase treatment followed by phenol chloroform extraction and ethanol precipitation. Viral DNA was then digested with HindIII and treated with Klenow fragment to end-label the restriction fragments with P33-dATP. The end-labeled restriction fragments were then size-fractionated by gel electrophoresis and visualized by autoradiography. The digestion products were compared with the digestion products from the pre-plasmid (that had been digested with Pme1/HindIII prior to labeling). The expected sizes were observed, indicating that the virus had been successfully rescued.

### EXAMPLE 8

Insertion of Ad5 Orf 6 into the E1 region of Ad24

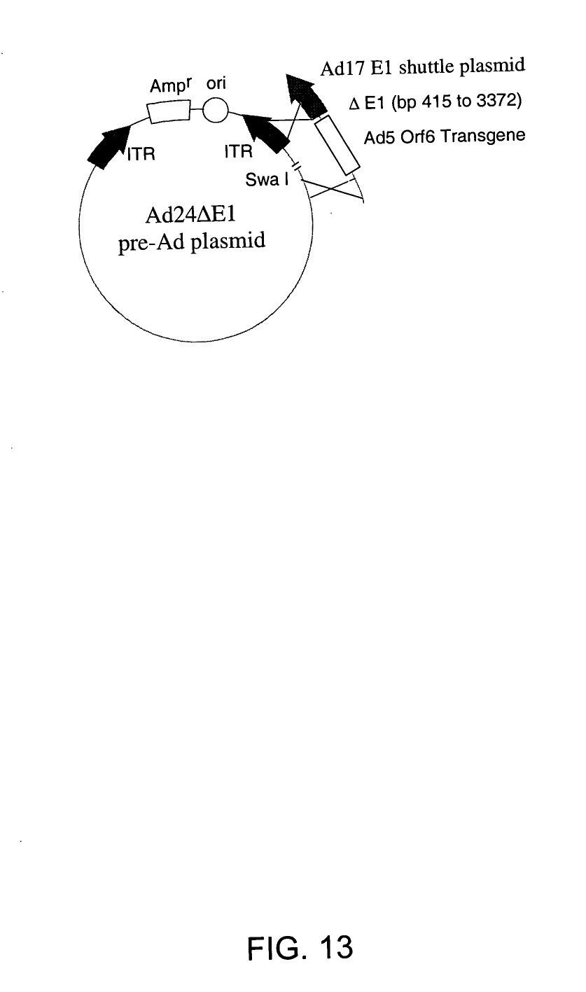

In order to determine if the insertion of Ad5 E4 Orf6 into the Ad24 genome would allow more efficient propagation in a group C E1 complementing cell line we constructed an Ad24 based pre-adenovirus plasmid containing Ad5 Orf6 in the E1 region. In order to introduce Ad5 Orf6 in to the E1 region of pAd24ΔE1, bacterial recombination was used. An Ad5 Orf6 transgene consisting of the Ad5 Orf6 coding region flanked by the HCMV promoter and pA was cloned into the E1 deletion in an Ad 17 shuttle vector (a precursor to the Ad 17 ITR cassette). The Ad5 Orf6 transgene was cloned between bp 414 and 3373 in the E1 anti-parallel orientation. The shuttle vector containing the Ad5 Orf6 transgene was digested to generate a DNA fragment consisting of the transgene flanked by Ad17 sequences (bp 4 to 414 and bp 3373 to 4580) and the fragment was purified after electrophoresis on an agarose gel. Cotransformation of BJ 5183 bacteria with the shuttle vector fragment and pAd24ΔE1, which had been linearized in the E1 region by digestion with SwaI, resulted in the generation of pAd24ΔE1Ad5Orf6 by homologous recombination (FIG. 13). Potential clones were screened by restriction analysis and one clone was selected as pre-adenovirus plasmid pAd24ΔE1Ad5Orf6.

In order to determine if pre-adenovirus plasmid pAd24ΔE1Ad5Orf6 could be rescued into virus and propagated in an Ad5/group C E1 complementing cell line, pAd24ΔE1Ad5Orf6 was digested with Pme I and transfected into a 6 cm dish of 293 cells using the calcium phosphate co-precipitation technique. PmeI digestion releases the viral genome from plasmid sequences allowing viral replication to occur after entry into 293 cells. Once complete viral cytopathic effect (CPE) was observed at approximately 7-10 days post transfection, the infected cells and media were freeze/thawed three times and the cell debris pelleted. The virus was amplified in two additional passages in 293 cells and then purified from the final infection by ultracentrifugation on CsCl density gradients. In order to verify the genetic structure of the virus, viral DNA was extracted using pronase treatment followed by phenol chloroform extraction and ethanol precipitation. Viral DNA was then digested with HindIII and treated with Klenow fragment to end-label the restriction fragments with P33-dATP. The end-labeled restriction fragments were then size-fractionated by gel electrophoresis and visualized by autoradiography. The digestion products were compared with the digestion products from the pre-plasmid (that had been digested with Pme1/HindIII prior to labeling). The expected sizes were observed, indicating that the virus had been successfully rescued.

### EXAMPLE 9

Insertion of Ad5 Orf6 into the E4 region of Ad24

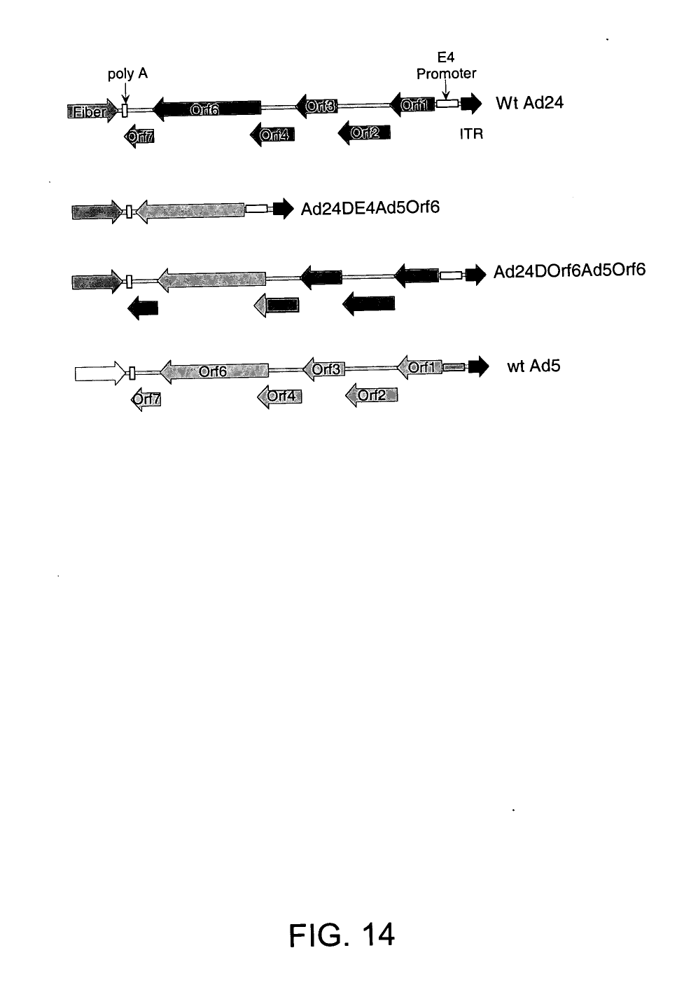

To refine the strategy of including Ad5 Orf6 in the genome of an alternative serotype so that propagation could take place in an Ad5/group C complementing cell line two additional strategies were developed. In the first strategy, the entire alternative serotype E4 region (not including the E4 promoter) was deleted and replaced with Ad5 Orf6. In the second strategy, just the alternative serotype Orf6 gene was deleted and replaced with Ad5 Orf6. The configuration of the E4 regions generated by the two strategies is diagramed in FIG. 14. For each of these strategies the desired pre-Adenovirus plasmid was generated by bacterial recombination. Cotransformation of BJ 5183 bacteria with pAd24ΔOrf6BstZ17I and the appropriately constructed Ad24 E4 shuttle plasmid resulted in the generation of the desired Ad24 based pre-Ad plasmid. PAd24ΔOrf6BstZ17I, a derivative of pAd24ΔE1, was constructed so that the E4 region in the Ad24 pre-Ad plasmid could be easily modified using bacterial recombination. PAd24ΔOrf6BstZ17I contains a deletion in the E4 region from Ad24 bp 32373 to bp 33328 with a unique BstZ17I site located at the position of the deletion. The complete sequence of pAd24ΔOrf6BstZ17I consists of Ad17 sequences from bp 4 to 414 and from bp 3373 to 4580, Ad24 bp 4588 to 32372 and from 33329 to 34529, and Ad17 bp 34469 to 35098 (bp numbers refer to the wt sequence for both Ad17 and Ad24).

To construct pAd24ΔE1ΔE4Ad5Orf6 (An Ad24 pre-Ad plasmid containing an E1 deletion and a deletion of E4 substituted with Ad5 Orf6), an Ad24 E4 shuttle plasmid was constructed by digesting pAd24ΔE1 with PmeI and BsrGI and cloning the restriction fragment representing the E4 region (bp 31559 to bp 35164) into pNEB193, generating pNEBAd24E4. PNEBAd24E4 was then digested with AccI and EcoNI to remove the E4 coding sequences and ligated with an oligo designed to contain BglII and XhoI sites (underlined) (5′ ACTCGAGATGTATAGATCT (SEQ ID NO: 6); 5′CTAGATCTATACATCTCGAG (SEQ ID NO: 7)), generating pNEBAd24ΔE4. PNEBAd24ΔE4 was then digested with BglII and XhoI and ligated with the Ad5 Orf6 gene, which was PCR amplified, generating pNEBAd24ΔE4Ad5Orf6. The PCR primers used to amplify the Ad5 Orf6 gene (5′ GCACAGATCTTTGCTTCAGGAATATG (SEQ ID NO: 8); 5′ GAGAACTCGAGGCCTACATGGGGGTAGAG (SEQ ID NO: 9)) were designed to contain BglII and XhoI sites (underlined above) for ligation with the pNEBAd24DE4 fragment. In the final step pNEBAd24ΔE4Ad5Orf6 E4 shuttle plasmid was digested with PvuI and PmeI, the restriction fragments were size fractionated by agarose gel electrophoresis and the desired fragment containing Ad5Orf6 flanked by Ad24 sequences was gel purified. Cotransformation of BJ 5183 bacteria with E4 shuttle fragment and pAd24ΔOrf6BstZ17I, which had been linearized in the E4 region by digestion with BstZ17I, resulted in the generation of pAd24ΔE1ΔE4Ad5Orf6 by homologous recombination. Potential clones were screened by restriction analysis and one clone was selected as pre-adenovirus plasmid pAd24ΔE1ΔE4Ad5Orf6.

To construct pAd24ΔE1ΔOrf6Ad5Orf6 (An Ad24 pre-Ad plasmid containing an E1 deletion and a deletion of E4 Orf6 substituted with Ad5 Orf6), an Ad24 E4 shuttle plasmid was constructed in which the Ad24 Orf6 gene was replaced by Ad5 Orf6. To do this the EcoRI restriction fragment representing bp 32126 to bp 33442 of the Ad24 genome (encompassing the E4 Orf6 coding region), was subcloned into the EcoRI site in pNEB193, generating pNEBAd24Orf6. In order to delete the E4 Orf6 gene in pNEBAd24Orf6 and replace it with Ad5 Orf6, pNEBAd24Orf6 was digested with StyI and treated with Klenow to blunt the ends and then digested with to EagI. The desired pNEBAd24Orf6 fragment was then ligated with a PCR product representing the Ad5 Orf6 gene from Ad5 bp 33193 to bp 24125, generating pNEBAd24ΔOrf6Ad5Orf6. The PCR primers used to generate the Ad5 Orf6 fragment (5′CGAGACGGCCGACGCAGATCTGTTTG (SEQ ID NO: 10); 5′GAAGTCCCGGGCTACATGGGGGTAG (SEQ ID NO: 11)) were designed to contain EagI and SmaI sites (underlined above) for ligation with the pNEBAd24Orf6 fragment. In the final step pNEBAd24ΔOrf6Ad5Orf6 was digested with EcoRI, the restriction fragments were size fractionated by agarose gel electrophoresis and the desired fragment containing Ad5Orf6 flanked by Ad24 sequences was gel purified. Cotransformation of BJ 5183 bacteria with the EcoRI fragment and pAd24ΔOrf6BstZ17I, which had been linearized in the E4 region by digestion with BstZ17I, resulted in the generation of pAd24ΔE1ΔOrf6Ad5Orf6 by homologous recombination. Potential clones were screened by restriction analysis and one clone was selected as pre-adenovirus plasmid pAd24ΔE1ΔOrf6Ad5Orf6.

### EXAMPLE 10

Rescue of pAd24ΔE1ΔE4Ad5Orf6, pAd24ΔE1ΔOrf6Ad5Orf6, into Virus

In order to determine if pre-adenovirus plasmids pAd24ΔE1ΔE4Ad5Orf6, pAd24ΔE1ΔOrf6Ad5Orf6, could be rescued into virus and propagated in a group C E1 complementing cell line, the plasmids were each digested with Pme I and transfected into T-25 flasks of PER.C6 cells using the calcium phosphate co-precipitation technique; (Cell Phect Transfection Kit, Amersham Pharmacia Biotech Inc.). PmeI digestion releases the viral genome from plasmid sequences allowing viral replication to occur after cell entry. Viral cytopathic effect (CPE), indicating that virus replication and amplification was occurring, was observed for both constructs. When CPE was complete, approximately 7-10 days post transfection, the infected cells and media were harvested, freeze/thawed three times and the cell debris pelleted by centrifugation. Approximately 1 ml of the cell lysate was used to infect T-225 flasks of PER.C6 cells at 80-90% confluence. Once CPE was reached, infected cells and media were harvested, freeze/thawed three times and the cell debris pelleted by centrifugation. Clarified cell lysates were then used to infect 2-layer NUNC cell factories of PER.C6 cells. Following complete CPE the virus was purified by ultracentrifugation on CsCl density gradients. In order to verify the genetic structure of the rescued viruses, viral DNA was extracted using pronase treatment followed by phenol chloroform extraction and ethanol precipitation. Viral DNA was then digested with HindIII and treated with Klenow fragment to end-label the restriction fragments with P33-dATP. The end-labeled restriction fragments were then size-fractionated by gel electrophoresis and visualized by autoradiography. The digestion products were compared with the digestion products of the corresponding pre-Adenovirus plasmid (that had been digested with Pme1/HindIII prior to labeling) from which they were derived. The expected sizes were observed, indicating that the viruses had been successfully rescued.

### EXAMPLE 11

Comparison of the Growth Kinetics of Ad24 Based Vectors.

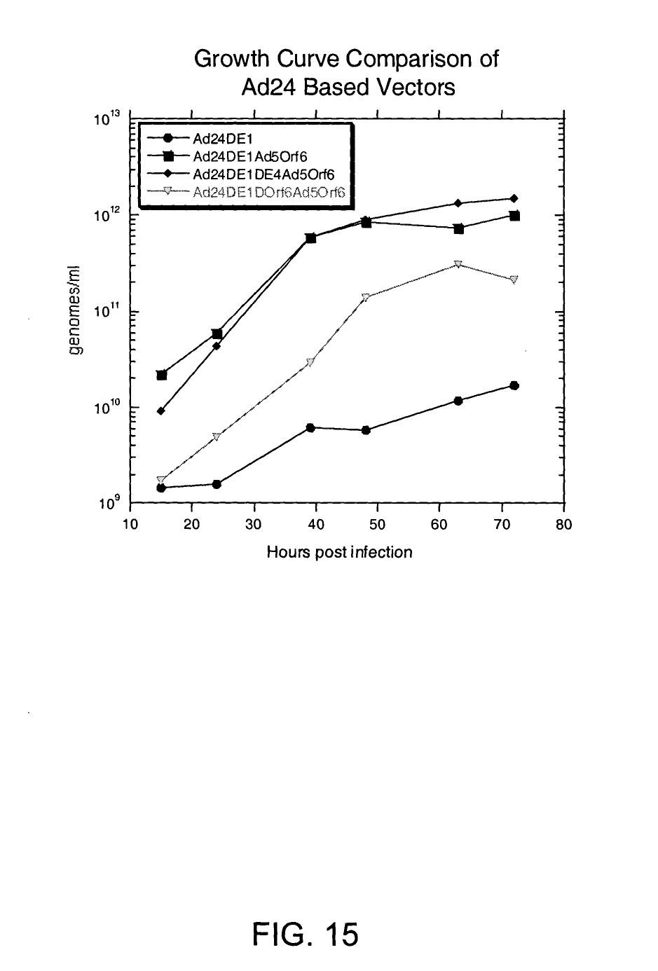

In order to compare the growth kinetic of Ad24ΔE1, Ad24ΔE1Ad5Orf6, Ad24ΔE1ΔE4Ad5Orf6 and Ad24ΔE1ΔOrf6Ad5Orf6 one step growth curves were preformed (FIG. 15). PER.C6 cells in 60 mm dishes were infected at 1 vp per cell with either Ad24ΔE1, Ad24ΔE1Ad5Orf6, Ad24ΔE1ΔE4Ad5Orf6 or Ad24ΔE1ΔOrf6Ad5Orf6. Cells and media were then harvested at various times post infection, freeze thawed three times and clarified by centrifugation. The amount of virus present in the samples was determined by quantitative PCR and is illustrated in FIG. 15. This study demonstrates that Ad24 vectors that incorporate Ad5 Orf6 have a distinct growth advantage over Ad24ΔE1 in PER.C6 cells. The instant invention can be practiced with recombinant Ad24 vectors absent a heterologous Orf 6 region where the E1-complementing cell line expresses an Ad24 μl region or, alternatively, E1 and E4 regions of the same serotype (such as Ad5E1/E4-expressing cell lines).

### EXAMPLE 12

Insertion of an Expression Cassette into pAd24ΔE1ΔE4Ad5Orf6, pAd24ΔE1ΔOrf6Ad5Orf6.

In order to introduce a gag or SEAP expression cassette (see FIGS. 6 and 7, respectively) into the E1 region of the Ad24 pre-Adenovirus plasmids described above (pAd24ΔE1ΔE4Ad5Orf6, pAd24ΔE1ΔOrf6Ad5Orf6) bacterial recombination was used. A gag expression cassette consisting of the following: 1) the immediate early gene promoter from the human cytomegalovirus, 2) the coding sequence of the human immunodeficiency virus type 1 (HIV-1) gag (strain CAM-1; 1526 bp) gene, and 3) the bovine growth hormone polyadenylation signal sequence, was cloned into the E1 deletion in Ad17 shuttle plasmid, pABSAd17-3, generating pABSAd17HCMVgagBGHpA. The ITR cassette contains sequences from the right (bp 34469 to 35098) and left (bp 4 to 414 and bp 3373 to 4580) end of the Ad17 genome separated by plasmid sequences containing a bacterial origin of replication and an Ampicillin resistance gene. The ITR cassette contains a deletion of E1 sequences from Ad17 (bp 415 to 3372) with a unique Swa I site located in the deletion. The gag expression cassette was obtained from a previously constructed shuttle plasmid by EcoRI digestion. Following the digestion the desired fragment was gel purified, treated with Klenow to obtain blunt ends and cloned into the SwaI site in pABSAd17-3. This cloning step resulted in the gag expression cassette being cloned into the E1 deletion between bp 414 and 3373 in the E1 parallel orientation. The shuttle vector containing the gag transgene was digested to generate a DNA fragment consisting of the gag expression cassette flanked by Ad17 bp 4 to 414 and bp 3373 to 4580 and the fragment was purified after electrophoresis on an agarose gel. Cotransformation of BJ 5183 bacteria with the shuttle vector fragment and one of the Ad24 pre-Ad plasmids (pAd24ΔE1ΔE4Ad5Orf6, pAd24ΔE1ΔOrf6Ad5Orf6,), linearized in the E1 region by digestion with Swa I, resulted in the generation of the corresponding Ad24 gag-containing pre-Adenovirus plasmids (pAd24ΔE1gagΔE4Ad5Orf6, pAd24ΔE1gagΔOrf6Ad5Orf6) by homologous recombination. Potential clones were screened by restriction analysis.

A similar strategy was used to generate Ad24 pre-Ad plasmids containing a SEAP expression cassette. In this case a SEAP expression cassette consisting of: 1) the immediate early gene promoter from the human cytomegalovirus, 2) the coding sequence of the human placental SEAP gene, and 3) the bovine growth hormone polyadenylation signal sequence was cloned into the E1 deletion in Ad17 shuttle plasmid, pABSAd17-3, generating pABSAd17HCMVSEAPBGH. The SEAP expression cassette was obtained from a previously constructed shuttle plasmid by EcoRI digestion. Following the digestion the desired fragment was gel purified, treated with Klenow to obtain blunt ends and cloned into the SwaI site in pABSAd17-3. The shuttle vector containing the SEAP transgene was digested to generate a DNA fragment consisting of the SEAP expression cassette flanked by Ad17 bp 4 to 414 and bp 3373 to 4580 and the fragment was purified after electrophoresis on an agarose gel. Cotransformation of BJ 5183 bacteria with the shuttle vector fragment and one of the Ad24 pre-Ad plasmids (pAd24ΔE1ΔE4Ad5Orf6, pAd24ΔE1ΔOrf6Ad5Orf6,), linearized in the E1 region by digestion with Swa I, resulted in the generation of the corresponding Ad24 SEAP-containing pre-Adenovirus plasmids (pAd24ΔE1SEAPΔE4Ad5Orf6, pAd24ΔE1 SEAPΔOrf6Ad5Orf6) by homologous recombination. Potential clones were screened by restriction analysis. All pre-Ad plasmids were rescued into virus and expanded to prepare CsCl purified stocks as described above.

### EXAMPLE 13

In Vivo Immunogenicity

A. Immunization

Cohorts of 3-6 animals were given intramuscular injections at wk 0 and wk 4 of either of the following constructs: (1) 10\hat{ }11 vp MRKAd5-HIV1 gag; (2) 10\hat{ }10 vp MRKAd5-HIV1 gag; (3) 10\hat{ }11 vp of Ad24ΔE1gagΔOrf6Ad5Orf6; (4) 10\hat{ }10 vp of Ad24ΔE1gagΔOrf6Ad5Orf6; or (5) 10\hat{ }10 vp of Ad24ΔE1gagΔE4Ad5Orf6. Rhesus macaques were between 3-10 kg in weight. In all cases, the total dose of each vaccine was suspended in 1 mL of buffer. The macaques were anesthetized (ketamine/xylazine) and the vaccines were delivered i.m. in 0.5-mL aliquots into both deltoid muscles using tuberculin syringes (Becton-Dickinson, Franklin Lakes, N.J.). Peripheral blood mononuclear cells (PBMC) were prepared from blood samples collected at several time points (typically 4 wk intervals) during the immunization regimen. All animal care and treatment were in accordance with standards approved by the Institutional Animal Care and Use Committee according to the principles set forth in the Guide for Care and Use of Laboratory Animals, Institute of Laboratory Animal Resources, National Research Council.

B. ELISPOT Assay

The IFN-γ ELISPOT assays for rhesus macaques were conducted following a previously described protocol (Allen et al., 2001 J. Virol. 75(2):738-749; Casimiro et al., 2002 J. Virol. 76:185-94), with some modifications. For antigen-specific stimulation, a peptide pool was prepared from 20-aa peptides that encompass the entire HIV-1 gag sequence with 10-aa overlaps (Synpep Corp., Dublin, Calif.). To each well, 50 μL of 2-4×10⁵ peripheral blood mononuclear cells (PBMCs) were added; the cells were counted using Beckman Coulter Z2 particle analyzer with a lower size cut-off set at 80 femtoliters (“fL”). Either 50 μL of media or the gag peptide pool at 8 μg/mL concentration per peptide was added to the PBMC. The samples were incubated at 37° C., 5% CO₂ for 20-24 hrs. Spots were developed accordingly and the plates were processed using custom-built imager and automatic counting subroutine based on the ImagePro platform (Silver Spring, Md.); the counts were normalized to 10⁶ cell input.

C. Intracellular Cytokine Staining

To 1 ml of 2×10⁶ PBMC/mL in complete RPMI media (in 17×100 mm round bottom polypropylene tubes (Sarstedt, Newton, N.C.)), anti-hCD28 (clone L293, Becton-Dickinson) and anti-hCD49d (clone L25, Becton-Dickinson) monoclonal antibodies were added to a final concentration of 1 μg/mL. For gag-specific stimulation, 10 μL of the peptide pool (at 0.4 mg/mL per peptide) were added. The tubes were incubated at 37° C. for 1 hr., after which 20 μL of 5 mg/mL of brefeldin A (Sigma) were added. The cells were incubated for 16 hr at 37° C., 5% CO₂, 90% humidity. 4 mL cold PBS/2% FBS were added to each tube and the cells were pelleted for 10 min at 1200 rpm. The cells were re-suspended in PBS/2% FBS and stained (30 min, 4° C.) for surface markers using several fluorescent-tagged mAbs: 20 μL per tube anti-hCD3-APC, clone FN-18 (Biosource); 20 μL anti-hCD8-PerCP, clone SK1 (Becton Dickinson); and 20 μL anti-hCD4-PE, clone SK3 (Becton Dickinson). Sample handling from this stage was conducted in the dark. The cells were washed and incubated in 750 μL 1×FACS Perm buffer (Becton Dickinson) for 10 min at room temperature. The cells were pelleted and re-suspended in PBS/2% FBS and 0.1 μg of FITC-anti-hIFN-γ, clone MD-1 (Biosource) was added. After 30 min incubation, the cells were washed and re-suspended in PBS. Samples were analyzed using all four color channels of the Becton Dickinson FACSCalibur instrument. To analyze the data, the low side- and forward-scatter lymphocyte population was initially gated; a common fluorescence cut-off for cytokine-positive events was used for both CD4⁺ and CD8⁺ populations, and for both mock and gag-peptide reaction tubes of a sample.

D. Anti-p24 ELISA

A modified competitive anti-p24 assay was developed using reagents from the Coulter p24 Antigen Assay kit (Beckman Coulter, Fullerton, Calif.). Briefly, to a 250-μL serum sample, 20 μL of Lyse Buffer and 15 μL of p24 antigen (9.375 pg) from the Coulter kit were added. After mixing, 200 μL of each sample were added to wells coated with a mouse anti-p24 mAb from the Coulter kit and incubated for 1.5 hr at 37° C. The wells were then washed and 200 μL of Biotin Reagent (polyclonal anti-p24-biotin) from the Coulter kit was added to each well. After a 1 hr, 37° C. incubation, detection was achieved using strepavidin-conjugated horseradish peroxidase and TMB substrate as described in the Coulter Kit. OD₄₅₀ₙₘ values were recorded. A 7-point standard curve was generated using a serial 2-fold dilution of serum from an HIV-seropositive individual. The lower cut-off for the assay is arbitrarily set at 10 milli Merck units/mL (mMU/mL) defined by a dilution of the seropositive human serum. This cutoff falls at approximately 65% of the maximum bound control signal which corresponds to that obtained with the diluent control only and with no positive analyte.

E. Results

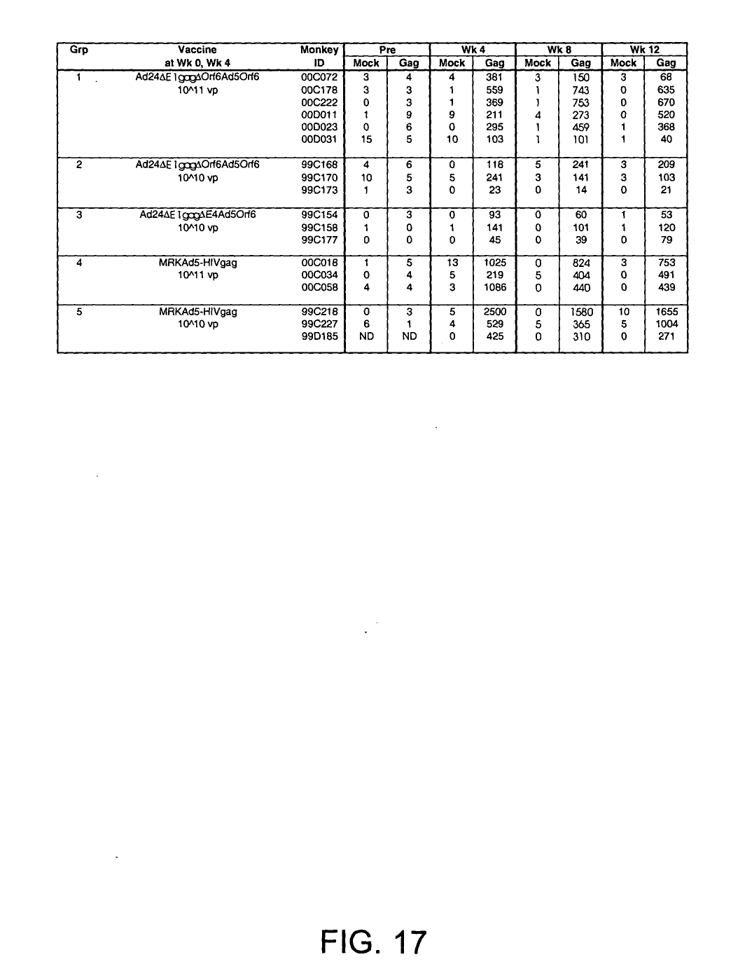

PBMCs collected at regular 4-wk intervals were analyzed in an ELISPOT assay (FIG. 17). Both Ad24ΔE1gagΔOrf6Ad5Orf6 and Ad24ΔE1gagΔE4Ad5Orf6 were able to induce significant levels of gag-specific T cells in non-human primates. At 10\hat{ }11 vp dose level, the Ad24-induced responses were within 2-3-fold of those of MRKAd5-HIV1 gag. Both Ad24 vectors were also able to induce detectable levels of gag-specific T cells at 10\hat{ }10 vp but were lower than those observed using MRKad5gag at the same dose.

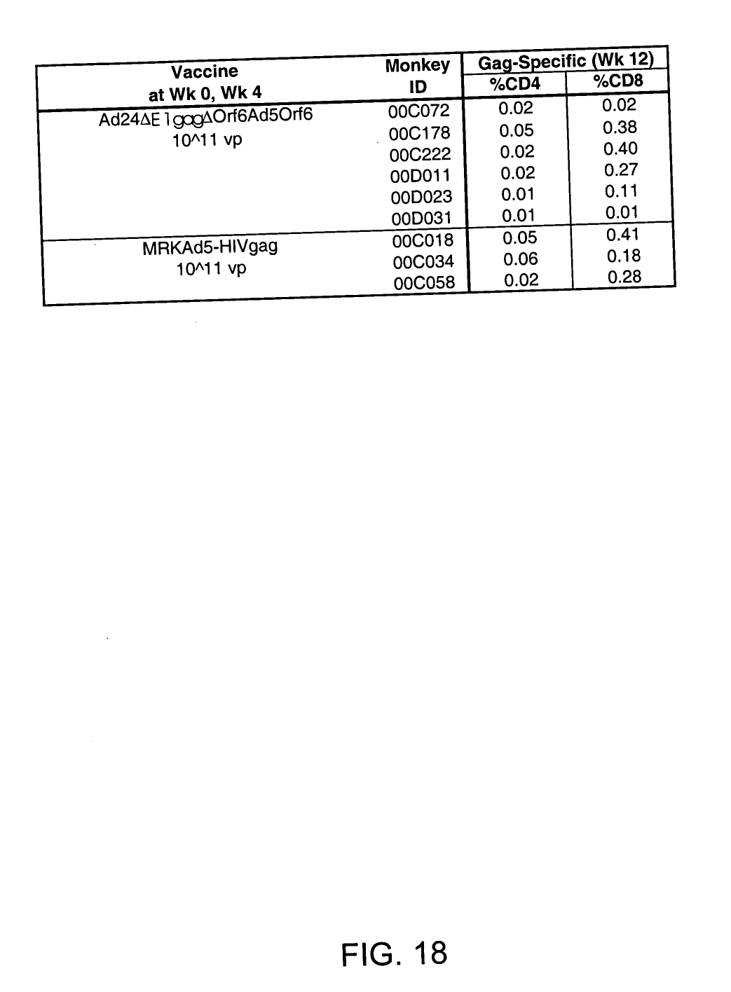

PBMCs collected at wk 12 from the vaccinees were analyzed for intracellular IFN-γ staining after the priming immunizations. The assay results provided information on the relative amounts of CD4⁺ and CD8⁺ gag-specific T cells in the peripheral blood (FIG. 18). The results indicated that the prime-boost immunization approach was able to elicit in rhesus macaques both HIV-specific CD4⁺ and CD8⁺ T cells.

F. Humoral Immune Responses

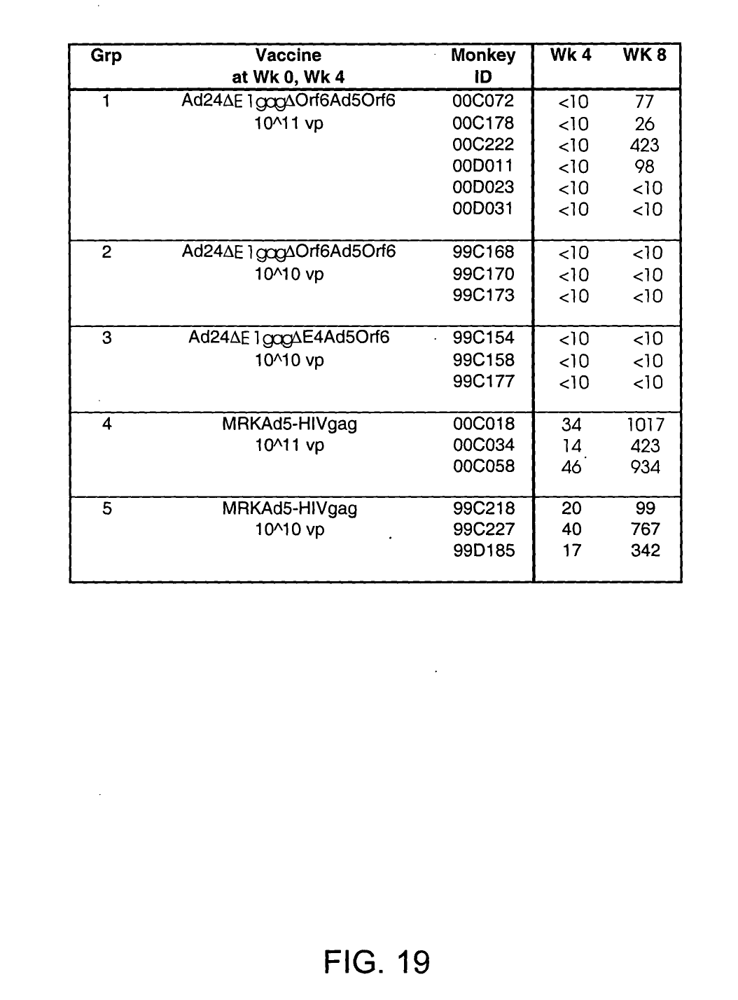

The Ad24-based vaccine vector was able to generate detectable levels of circulating anti-gag antibodies at the reasonably high dose level (FIG. 19). No detectable titers were observed at equal to or lower than 10\hat{ }10 vp, suggesting the existence of a dose-dependent response.

### EXAMPLE 14

In Vivo Transgene Expression

A. Immunization

Cohorts of 5 C3H/HeN mice were given single intramuscular injections of one of the following vectors: (1) 10\hat{ }10 vp Ad24ΔE1SEAPΔE4Ad5Orf6; (2) 10\hat{ }10 vp Ad24ΔE1SEAPΔOrf6Ad5Orf6; (3) 10\hat{ }10 vp MRKAd5SEAP; and (4) 10\hat{ }9 vp MRKAd5SEAP. Female mice were between 4-10 weeks old. The total dose of each vaccine was suspended in 0.1 mL of buffer. The vectors were given to both quadriceps of each of the animals with a volume of 50 uL per quad and using 0.3-mL 28G1/2 insulin syringes (Becton-Dickinson, Franklin Lakes, N.J.). For the primates, the total dose of each vaccine was suspended in 1 mL of buffer. The monkeys were anesthetized (ketamine/xylazine mixture) and the vaccines were delivered i.m. in 0.5-mL aliquots into two muscle sites using tuberculin syringes (Becton-Dickinson, Franklin Lakes, N.J.). Serum samples were collected at defined intervals and stored frozen until the assay date. All animal care and treatment were in accordance with standards approved by the Institutional Animal Care and Use Committee according to the principles set forth in the Guide for Care and Use of Laboratory Animals, Institute of Laboratory Animal Resources, National Research Council.

B. SEAP Assay

Serum samples were analyzed for circulating SEAP levels using TROPIX phospha-light chemiluminescent kit (Applied Biosystems Inc). Duplicate 5 uL aliquots of each serum were mixed with 45 uL of kit-supplied dilution buffer in a 96-well white DYNEX plate. Serially diluted solutions of a human placental alkaline phosphatase (Catalog no. M5905, Sigma, St. Louis, Mo.) in 10% naive monkey serum served to provide the standard curve. Endogenous SEAP activity in the samples was inactivated by heating the wells for 30 minutes at 65° C. Enzymatic SEAP activities in the samples were determined following the procedures described in the kit. Chemiluminescence readings (in relative light units) were recorder using DYNEX luminometer. RLU readings are converted to ng/mL SEAP using a log-log regression analyses.

C. Rodent Results

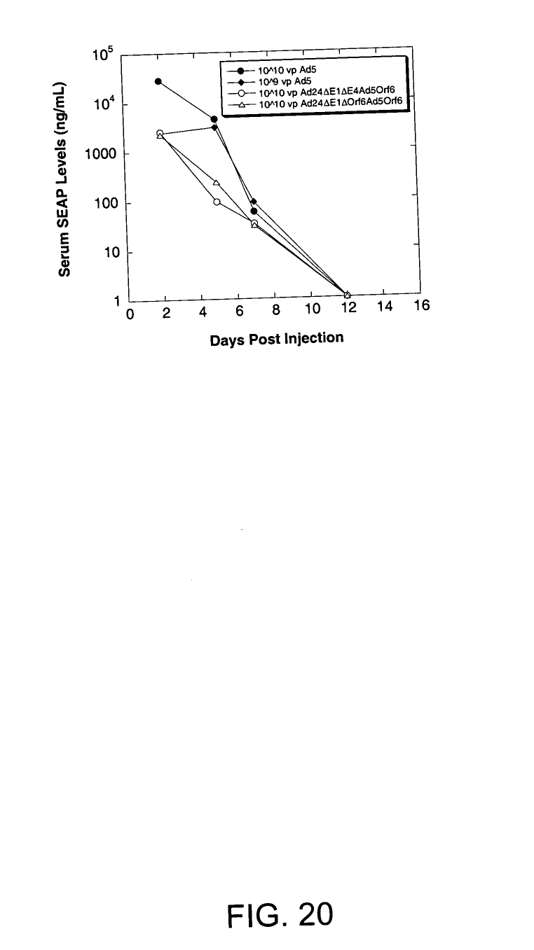

Serum samples prior to and after the injection were analyzed for circulating SEAP activities and the results are shown in FIG. 20. Results indicate that (1) both Ad24 constructs are all capable of expressing the SEAP transgene in vivo to comparable levels; and that (2) the level of expression achieved using the Ad24 vectors are comparable to that of Ad5 at 10-fold lower dose. The levels of SEAP in the serum dropped dramatically after day 2 and were at background levels by day 12.

D. Primate Results

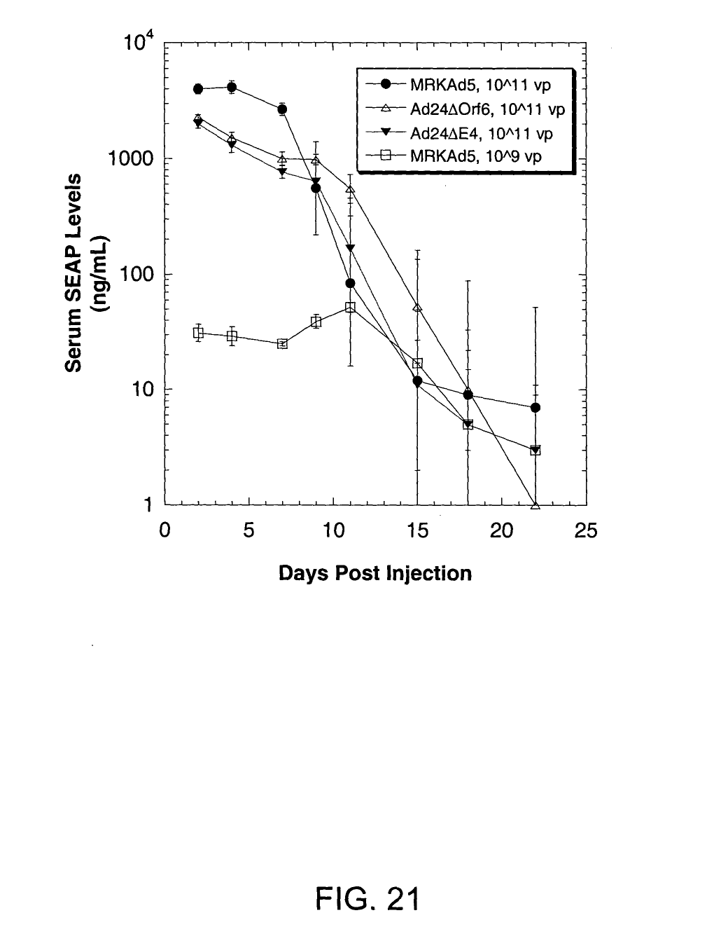

Cohorts of 3 rhesus macaques were given single intramuscular injections of one of the following vectors: (1) 10\hat{ }11 vp MRKAd5-SEAP; (2) 10\hat{ }9 vp MRKAd5-SEAP; (3) 10\hat{ }11 vp Ad24ΔE1SEAPΔOrf6Ad5Orf6; or (4) 10\hat{ }11 vp Ad24ΔE1SEAPΔE4Ad5Orf6. Serum samples prior to and after the injection were analyzed for circulating SEAP activities and the results are shown in FIG. 21.

Results indicate that the peak levels of SEAP product produced by adenovirus serotype 24 were lower than but were within 3-fold of that of MRKAd5 at the same high dose level of 10\hat{ }11 vp (FIG. 21). The levels observed with adenovirus serotype 24 are generally 50-fold higher than those observed using 10\hat{ }9 vp of MRKAd5. The levels of SEAP in the serum dropped dramatically after day 10 and were close to background as early as day 15. These observations strongly indicate that adenovirus serotype 24 is very efficient in expressing a transgene following intramuscular administration in a primate.

### EXAMPLE 15

Construction of pMRKAd24ΔE1ΔE4Ad5Orf6

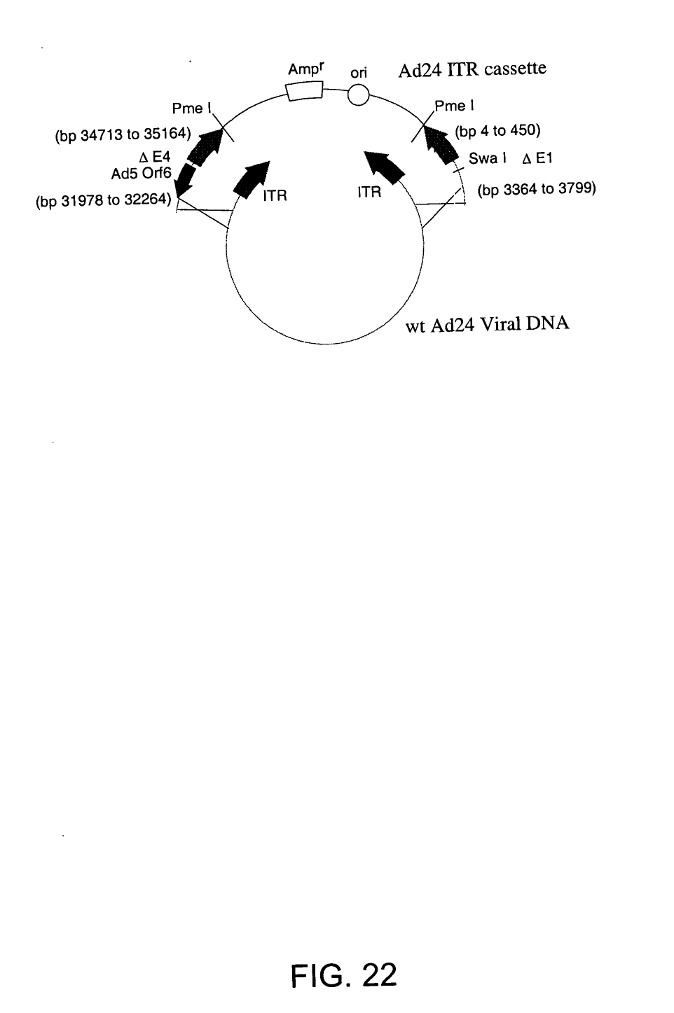

To construct pMRKAd24ΔE1ΔE4Ad5Orf6 (An Ad24 pre-Ad plasmid, composed entirely of Ad24 sequence and containing an E1 deletion and an E4 deletion substituted with Ad5 Orf6), an Ad24 ITR cassette was constructed containing sequences from the right (bp 31978 to 32264 and bp 34713 to 35164) and left (bp 4 to 450 and bp 3364 to 3799) end of the Ad24 genome separated by plasmid sequences containing a bacterial origin of replication and an ampicillin resistance gene. These four segments were generated by PCR and cloned sequentially into pNEB193, generating pNEBAd24-4. Next the Ad5 Orf6 open reading frame (Ad5 bp 31192 to bp 34078) was generated by PCR and cloned between Ad24 bp 32264 and 34713 generating pNEBAd24E-Ad5Orf6 (the ITR cassette). PNEB193 is a commonly used commercially available cloning plasmid (New England Biolabs cat# N3051S) containing a bacterial origin of replication, ampicillin resistance gene and a multiple cloning site into which the PCR products were introduced. The ITR cassette contains a deletion of E1 sequences from Ad24 bp 451 to 3363 with a unique Swa I restriction site located in the deletion and an E4 deletion from Ad24 bp 32265 to 34712 into which Ad5 Orf6 was introduced in an E4 parallel orientation. In this construct Ad5 Orf6 expression is driven by the Ad24 E4 promoter. The Ad24 sequences (bp 31978 to 32264 and bp 3464 to 3799) in the ITR cassette provide regions of homology with the purified Ad24 viral DNA in which bacterial recombination can occur following cotransformation into BJ 5183 bacteria (FIG. 22). The ITR cassette was also designed to contain unique restriction enzyme sites (PmeI) located at the end of the viral ITR's so that digestion will release the recombinant Ad24 genome from plasmid sequences. Potential clones will be screened by restriction analysis and one clone was selected as pMRKAd24ΔE1ΔE4Ad5Orf6. Pre-Adenovirus plasmid pMRKAd24ΔE1ΔE4Ad5Orf6 should contain Ad24 sequences from bp 4 to 450; bp 3364 to bp 32264 and bp 34713 to bp 35164 with Ad5Orf6 cloned between bp 32264 and bp 34713. The bp numbering in the above description refers to the wt sequence for both Ad24 and Ad5.

### EXAMPLE 16

Insertion of HIV-1 gag and SEAP Transgenes into pAd24ΔE1ΔE4Ad5Orf6

In order to introduce a gag or SEAP expression cassettes into the E1 region of pMRKAd24ΔE1ΔE4Ad5Orf6, bacterial recombination will be used. An HIV-1 gag expression cassette will consist of the following: 1) the immediate early gene promoter from the human cytomegalovirus, 2) the coding sequence of the human immunodeficiency virus type 1 (HIV-1) gag (strain CAM-1; 1526 bp) gene, and 3) the bovine growth hormone polyadenylation signal sequence, in the E1 deletion of an Ad24 shuttle plasmid, pNEBAd24-2 (a precursor to the Ad24 ITR cassette described above), generating pNEBAd24CMVgagBGHpA. PNEBAd24-2 contains Ad24 sequences from the left end of the genome (bp 4 to 450 and bp 3364 to 3799) that define the E1 deletion. The gag expression cassette will be obtained from a previously constructed plasmid and cloned into the E1 deletion between bp 450 and 3364 in the E1 parallel orientation. The shuttle vector containing the gag transgene will be digested to generate a DNA fragment consisting of the gag expression cassette flanked by Ad24 bp 4 to 450 and bp 3364 to 3799 and the fragment will be purified after electrophoresis on an agarose gel. Cotransformation of BJ 5183 bacteria with the shuttle vector fragment and pMRKAd24ΔE1ΔE4Ad5Orf6 which was linearized in the E1 region by digestion with SwaI, should result in the generation of Ad24 gag-containing pre-Adenovirus plasmids pMRKAd24ΔE1gagΔE4Ad5Orf6 by homologous recombination. Potential clones will be screened by restriction analysis.

A similar strategy will be used to generate Ad24 pre-Ad plasmids containing a SEAP expression cassette. In this case, a SEAP expression cassette will consist of: 1) the immediate early gene promoter from the human cytomegalovirus, 2) the coding sequence of the human placental SEAP gene, and 3) the bovine growth hormone polyadenylation signal sequence cloned into the E1 deletion of an Ad24 shuttle plasmid, pNEBAd24-2, generating pNEBAd24CMVSEAPBGHpA. The transgene will then be recombined into pMRKAd24ΔE1ΔE4Ad5Orf6 as described above for the gag transgene.

### EXAMPLE 17

In Vivo Immunogenicity

A. Immunization

Rhesus macaques were between 3-10 kg in weight. In all cases, the total dose of each vaccine was suspended in 1 mL of buffer. The macaques were anesthetized (ketamine/xylazine) and the vaccines were delivered i.m. in 0.5-mL aliquots into both deltoid muscles using tuberculin syringes (Becton-Dickinson, Franklin Lakes, N.J.). Peripheral blood mononuclear cells (PBMC) were prepared from blood samples collected at several time points during the immunization regimen. All animal care and treatment were in accordance with standards approved by the Institutional Animal Care and Use Committee according to the principles set forth in the Guide for Care and Use of Laboratory Animals, Institute of Laboratory Animal Resources, National Research Council.

B. T Cell Responses

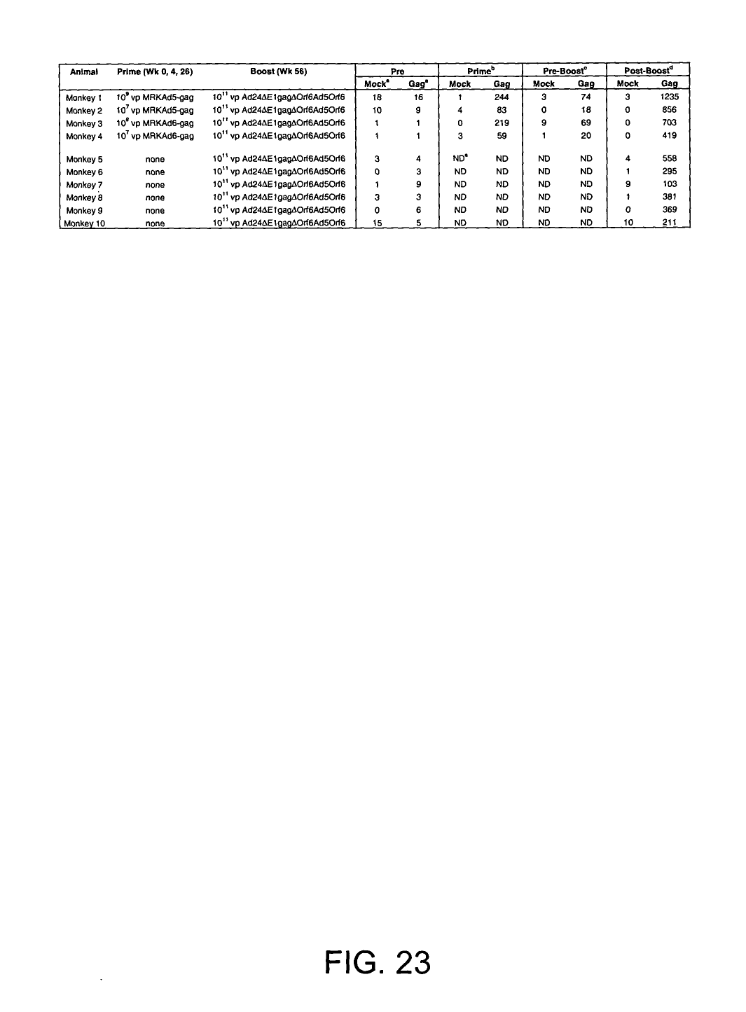

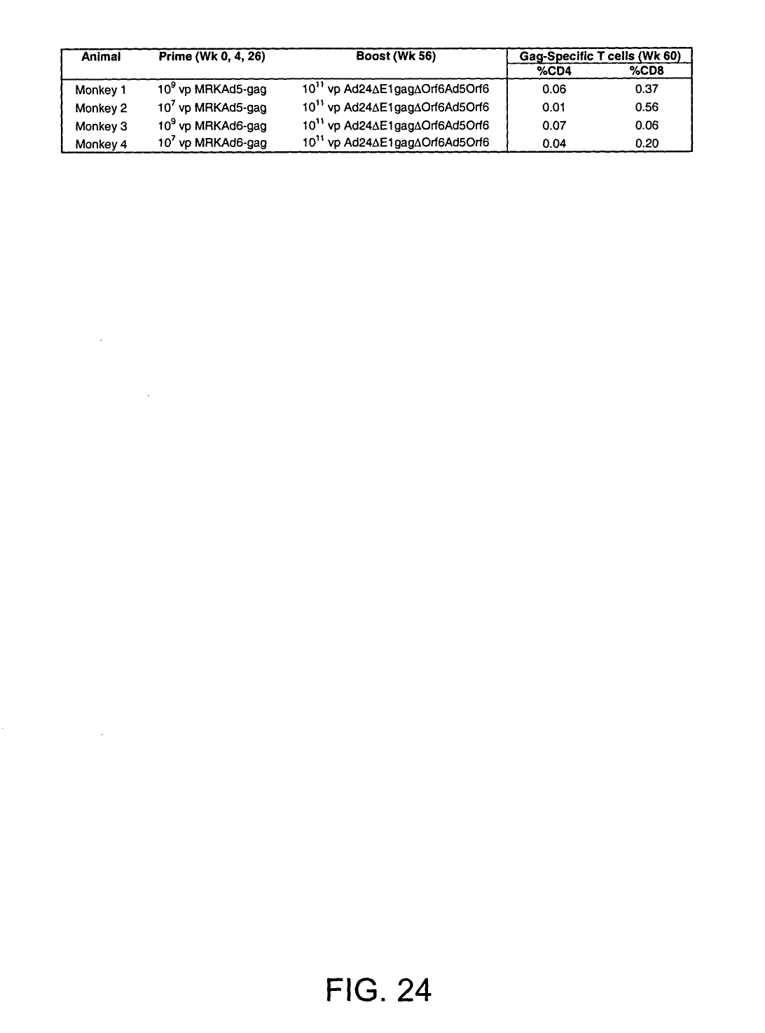

Ad24 Vaccine Vector as a Heterologous Booster: Cohort of 4 rhesus macaques was immunized initially with 3 doses (wk 0, 4, 26) of either 10⁷ or 10⁹ vp of MRKAd5-gag (see, PCT/US01/28861, published Mar. 21, 2002) or MRKAd6-gag. At wk 56, the animals received a booster vaccine of 10¹¹ vp Ad24ΔE1gagΔOrf6Ad5Orf6. A separate cohort of naïve animals received a single dose of the booster vaccine. The results of the IFN-γ ELISPOT analyses of PBMC collected during the course of the studies are shown in FIG. 23. It is apparent that the Ad24 HIV vectors can be utilized to amplify the existing pools of HIV-specific T cells. The increases in the levels of gag-specific T cells from the pre-boost levels to those measured at 4 wks post boost were consistently larger than the levels induced by the same booster vaccine in naive animals. PBMCs from the vaccinees of the heterologous MRKAd5/MRKAd6-Ad24 boost regimen were analyzed for intracellular IFN-γ staining after the priming immunizations (wk 60). The assay results provided information on the relative amounts of CD4⁺ and CD8⁺ gag-specific T cells in the peripheral blood (FIG. 24). The results indicated that heterologous prime-boost immunization approach was able to elicit in rhesus macaques both HIV-specific CD4+ and CD8+ T cells.

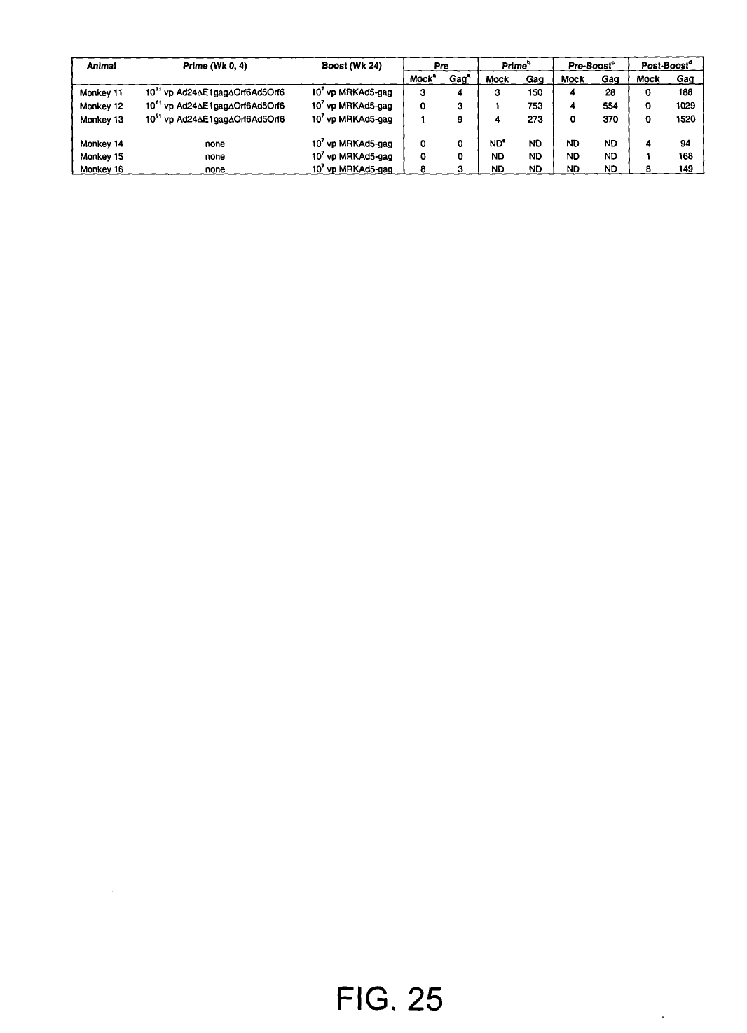

Ad24 Vaccine Vector as a Heterologous Primer: In a separate study, a cohort of 3 rhesus macaques was immunized initially with 2 doses (wk 0, 4) of 10¹¹ vp Ad24ΔE1gagΔOrf6Ad5Orf6 and boosted at wk 24 with 10⁷ vp of MRKAd5-gag. The low dose of MRKAd5-gag is selected to mimic the effect of pre-existing neutralizing immunity to the vector in a subject. A separate cohort of naïve animals was given a single dose of 10⁷ vp MRKAd5-gag. The results of the IFN-γ ELISPOT analyses of PBMC collected during the course of the studies are shown in FIG. 25.

The Ad24-based vaccine was able to prime effectively for HIV-specific T cell responses in macaques. Boosting with a low dose MRKAd5-gag resulted in a significant increase in the levels of gag-specific T cells. The increases in 2 out of 3 animals exceed the levels typically observed after treatment of naïve animals with the same low dose of MRKAd5-gag.

### EXAMPLE 18

Construction of pAd34ΔE1ΔE4Ad5Orf6

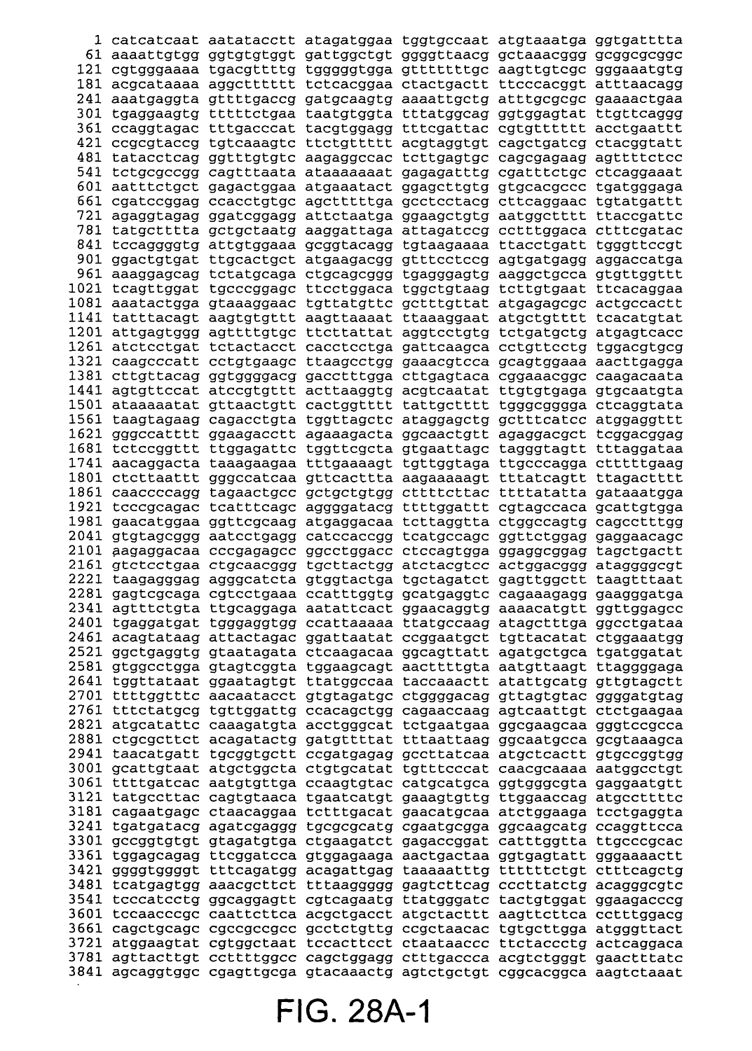

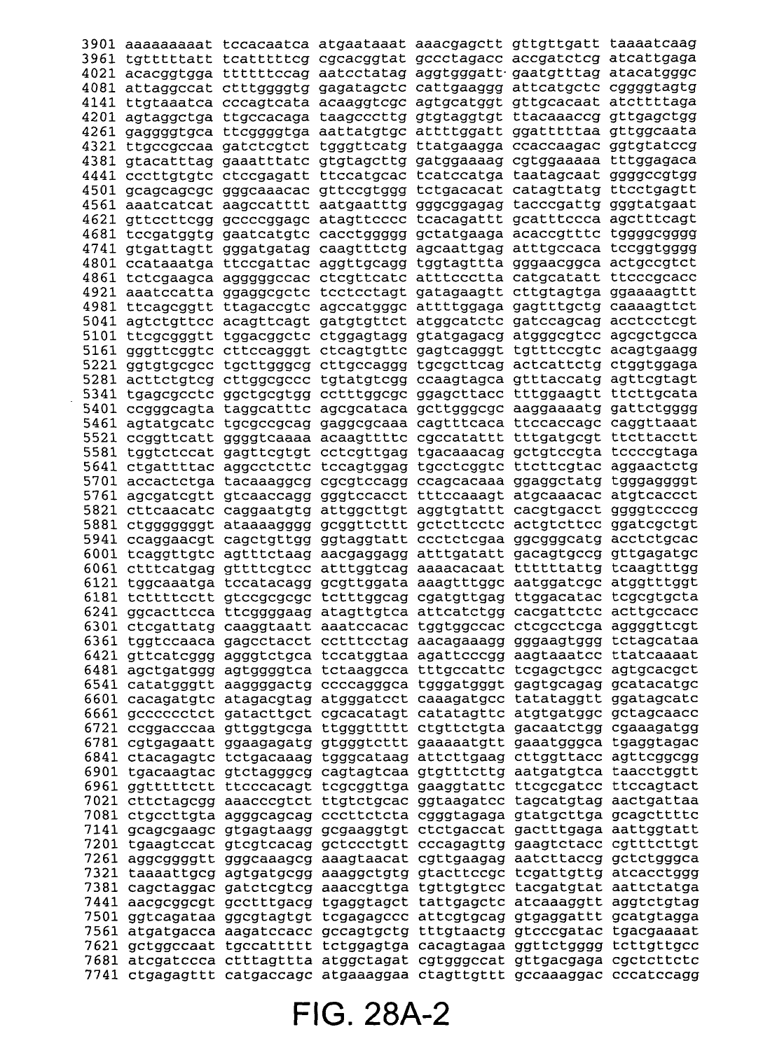

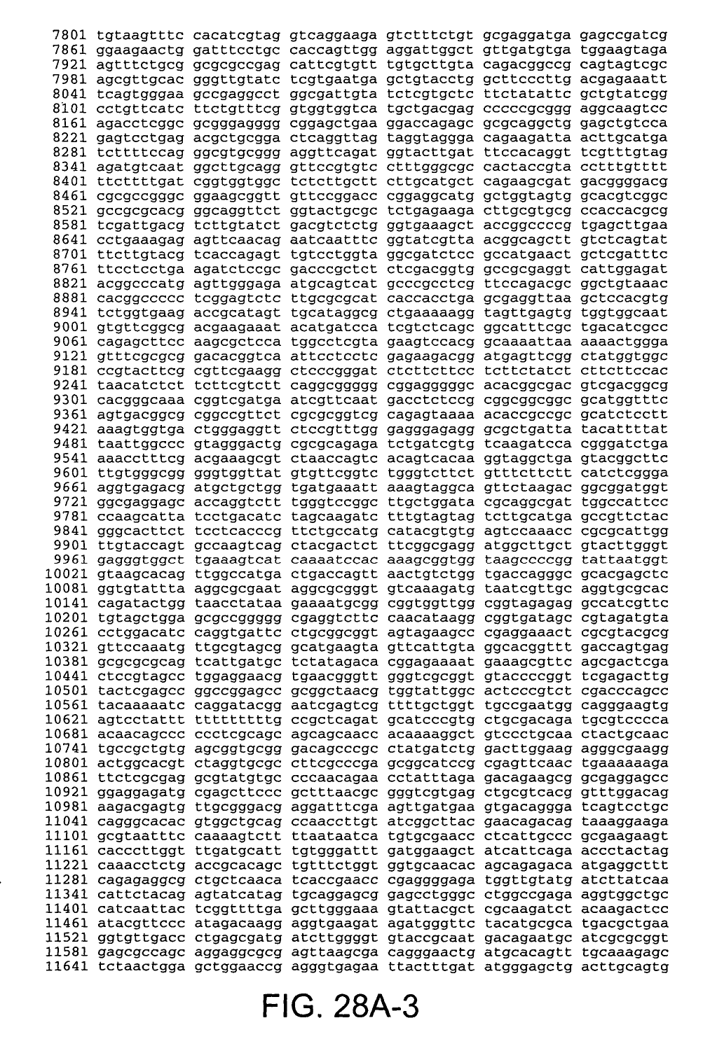

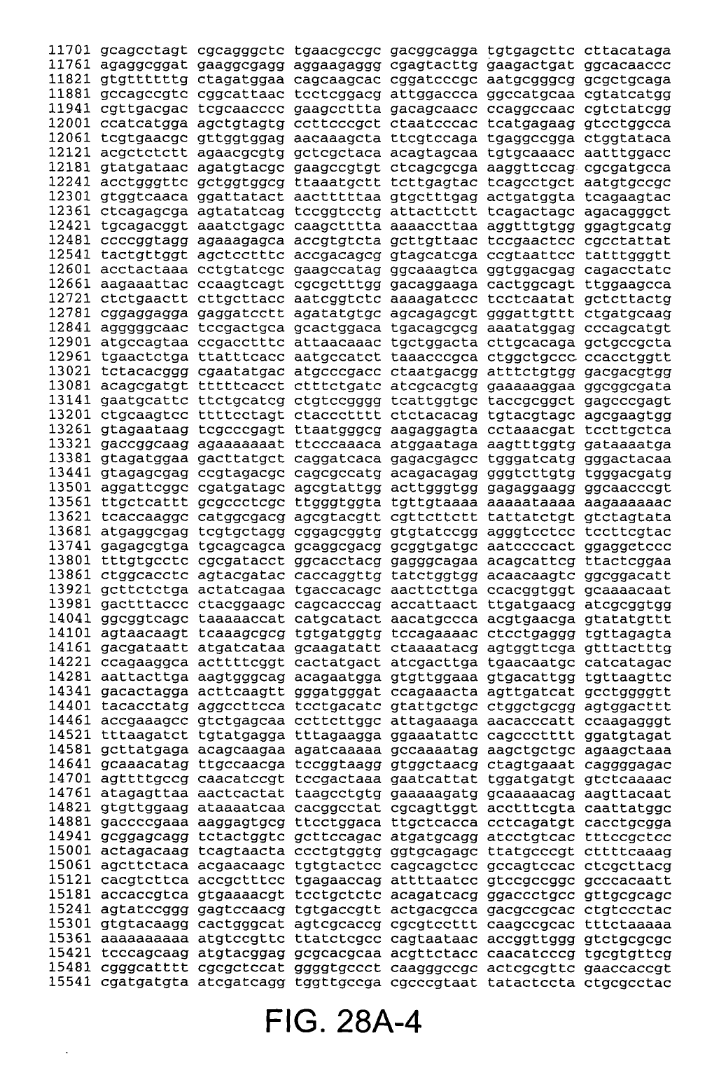

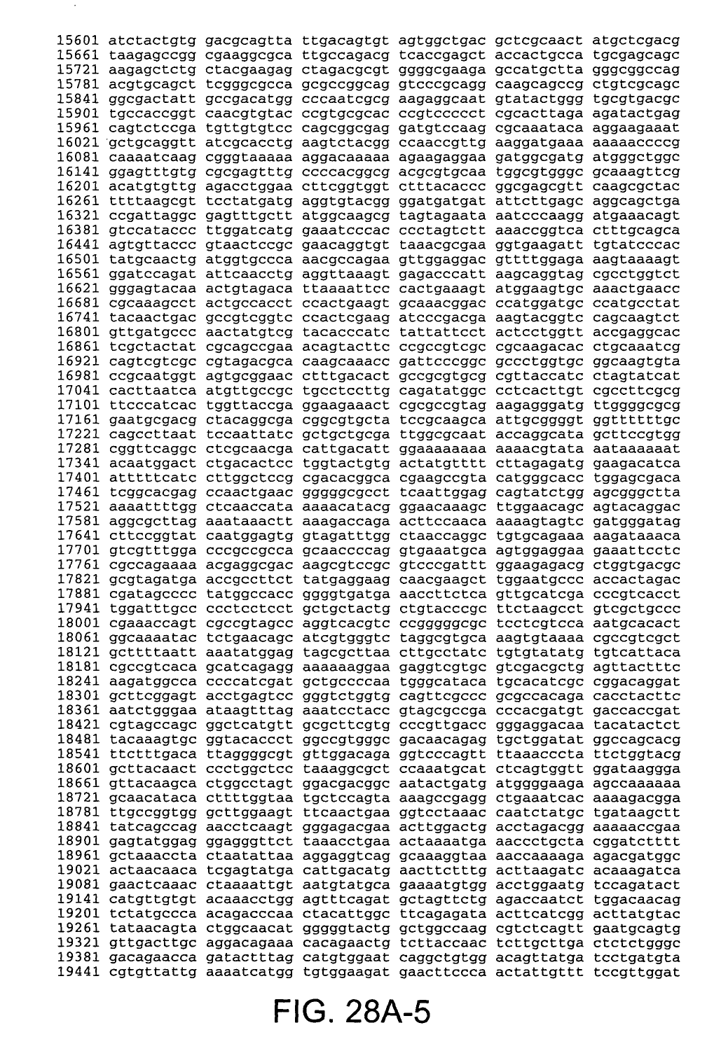

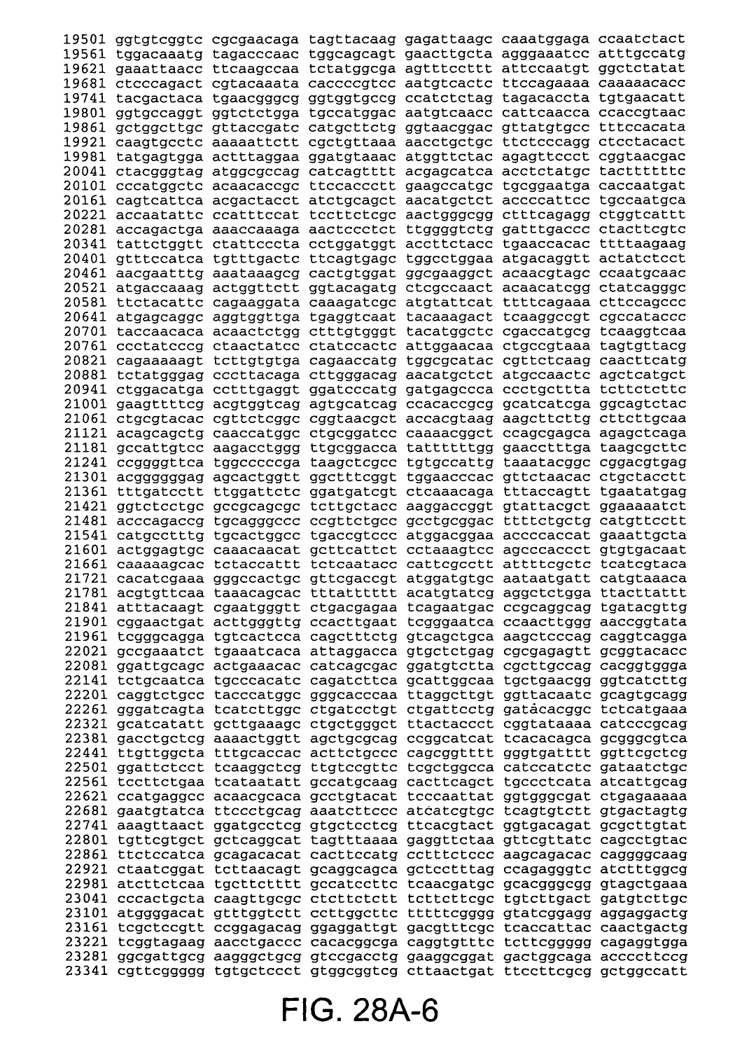

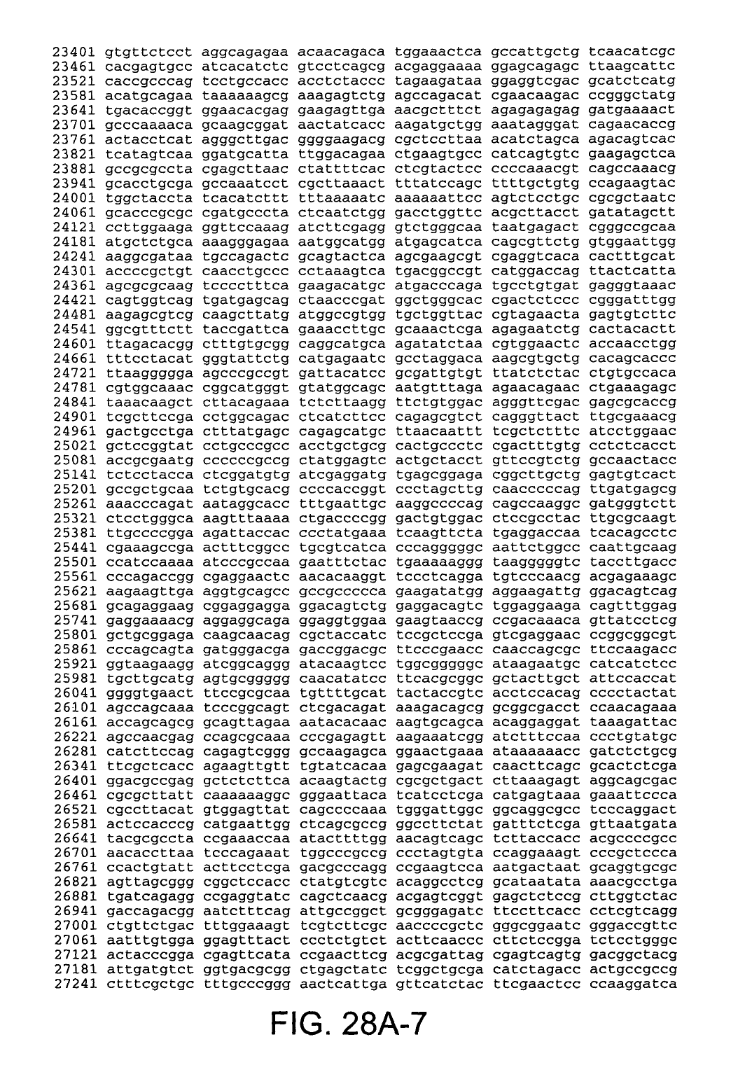

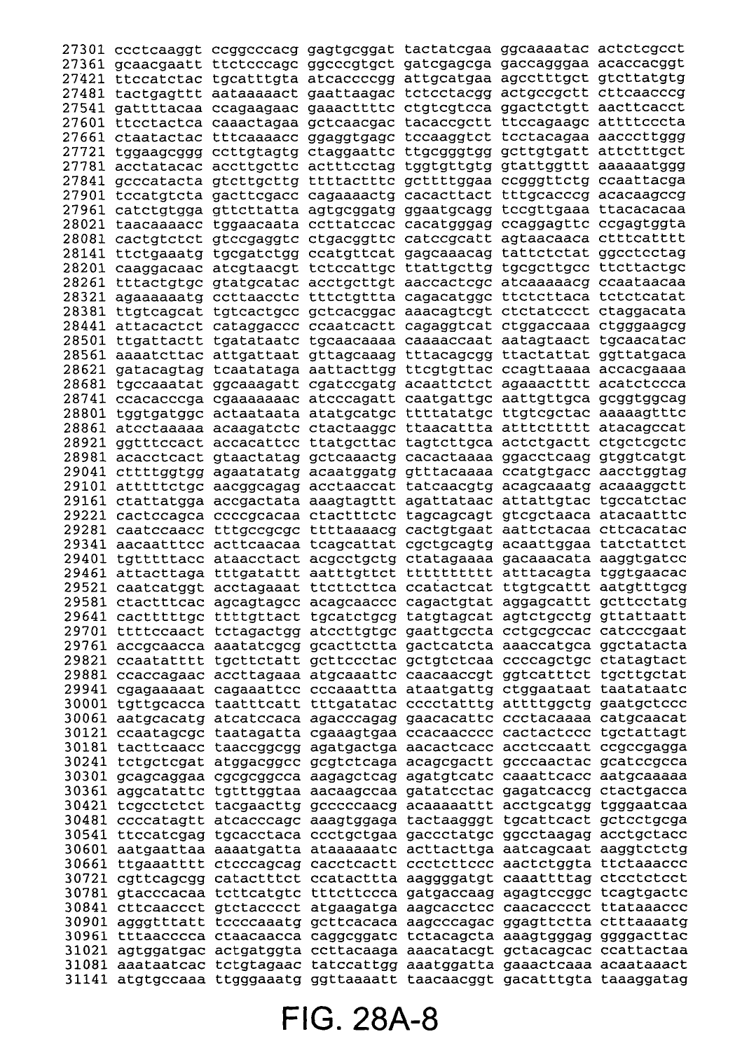

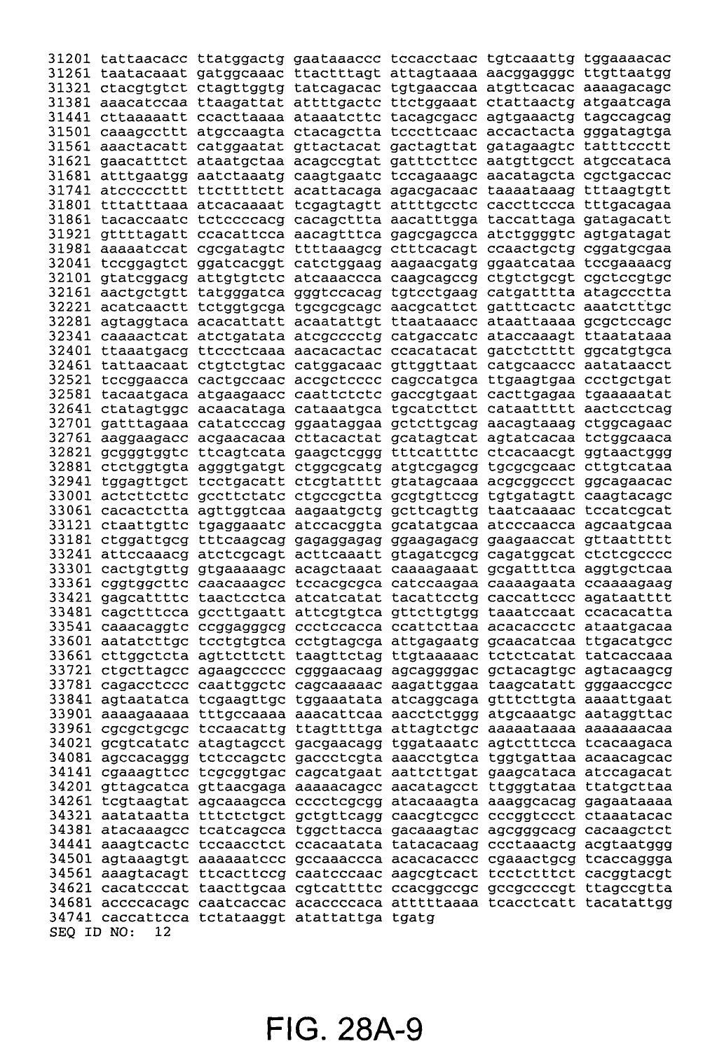

To generate an E1-Ad34 based vector that can propagate in existing group C/Ad5 E1 complementing cell lines (293, PER.C6), Ad5 Orf6 was inserted in place of the native E4 region. Since at the time, the complete sequence of Ad34 (see FIGS. 28A-1 to 28A-9; subject of copending application serial No. 60/458,825, filed Mar. 28, 2003) was unknown, advantage was taken of the sequence homology between Ad34 and Ad35 in order to construct the Ad34 pre-Adenovirus plasmid. Cotransformation of BJ 5183 bacteria with purified wild-type Ad34 viral DNA and the appropriately constructed Ad35 ITR cassette resulted in the circularization of the viral genome by homologous recombination. The construction of the pre-Ad plasmid based on Ad34, is outlined below:

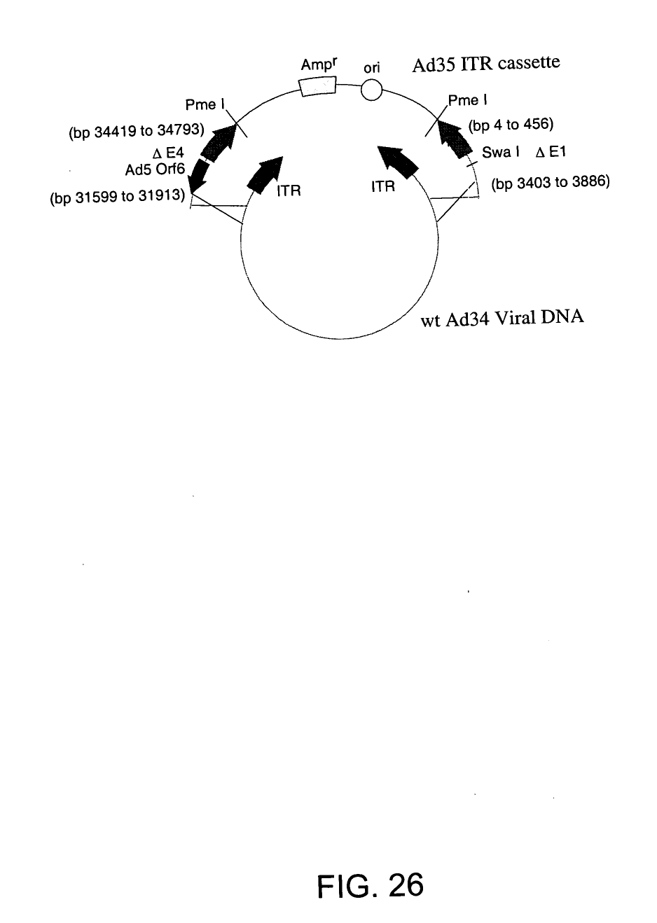

To construct pAd34ΔE1ΔE4Ad5Orf6 (An Ad34 pre-Ad plasmid containing an E1 deletion and an E4 deletion substituted with Ad5 Orf6), we utilized an Ad35 ITR cassette. We anticipated that sequence homology between Ad34 and Ad35 would allow homologous recombination to occur. The Ad35 ITR cassette was constructed containing sequences from the right (bp 31599 to 31913 and bp 34419 to 34793) and left (bp 4 to 456 and bp 3403 to 3886) end of the Ad35 genome (see FIGS. 2A-1 to 2A-10) separated by plasmid sequences containing a bacterial origin of replication and an ampicillin resistance gene. The four segments were generated by PCR and cloned sequentially into pNEB193, generating pNEBAd35-4. Next the Ad5 Orf6 open reading frame was generated by PCR and cloned between Ad35 bp 31913 and 34419 generating pNEBAd35-4Ad5Orf6 (the ITR cassette). PNEB193 is a commonly used commercially available cloning plasmid (New England Biolabs cat# N3051S) containing a bacterial origin of replication, ampicillin resistance gene and a multiple cloning site into which the PCR products were introduced. The ITR cassette contains a deletion of E1 sequences from Ad35 bp 457 to 3402 with a unique Swa I restriction site located in the deletion and an E4 deletion from Ad35 bp 31914 to 34418 into which Ad5 Orf6 was introduced in an E4 parallel orientation. In this construct Ad5Orf6 expression is driven by the Ad35 E4 promoter. The Ad35 sequences (bp 31599 to 31913 and bp 3403 to 3886) in the ITR cassette provided regions of homology with the purified Ad34 viral DNA in which bacterial recombination could occur following cotransformation into BJ 5183 bacteria (FIG. 26). The ITR cassette was also designed to contain unique restriction enzyme sites (PmeI) located at the end of the viral ITR's so that digestion would release the recombinant Ad34 genome from the plasmid sequences. Potential clones were screened by restriction analysis and one clone was selected as pAd34ΔE1ΔE4Ad5Orf6.

### EXAMPLE 19

Rescue of pAd34ΔE1ΔE4Ad5Orf6 into Virus

In order to determine if pre-adenovirus plasmid pAd34ΔE1ΔE4Ad5Orf6, could be rescued into virus and propagated in a group C E1 complementing cell line, the plasmid was digested with Pme I and transfected into T-25 flasks of PER.C6 cells using the calcium phosphate co-precipitation technique (Cell Phect Transfection Kit, Amersham Pharmacia Biotech Inc). PmeI digestion releases the viral genome from plasmid sequences allowing viral replication to occur after cell entry. Viral cytopathic effect (CPE), indicating that virus replication and amplification was occurring was observed following transfection. When CPE was complete, approximately 7-10 days post transfection, the infected cells and media were harvested, freeze/thawed three times and the cell debris pelleted by centrifugation. Approximately 1 ml of the cell lysate was used to infect a T-225 flask of PER.C6 cells at 80-90% confluence. Once CPE was reached, infected cells and media were harvested, freeze/thawed three times and the cell debris pelleted by centrifugation. Clarified cell lysates were then used to infect 2-layer NUNC cell factories of PER.C6 cells. Following complete CPE, the virus was purified by ultracentrifugation on CsCl density gradients. In order to verify the genetic structure of the rescued viruses, viral DNA was extracted using pronase treatment followed by phenol chloroform extraction and ethanol precipitation. Viral DNA was then digested with HindIII and treated with Klenow fragment to end-label the restriction fragments with P33-dATP. The end-labeled restriction fragments were then size-fractionated by gel electrophoresis and visualized by autoradiography. The digestion products were compared with the digestion products of the corresponding pre-Adenovirus plasmid (that had been digested with Pme1/HindIII prior to labeling) from which they were derived. The expected sizes were observed, indicating that the viruses had been successfully rescued.

### EXAMPLE 20

Insertion of an Expression Cassette into pAd34ΔE1ΔE4Ad5Orf6

In order to introduce a gag or SEAP expression cassette (see FIGS. 6 and 7, respectively) into the E1 region of pAd34ΔE1ΔE4Ad5Orf6, bacterial recombination was again used. A gag expression cassette consisting of the following: 1) the immediate early gene promoter from human cytomegalovirus, 2) the coding sequence of the human immunodeficiency virus type 1 (HIV-1) gag (strain CAM-1; 1526 bp) gene, and 3) the bovine growth hormone polyadenylation signal sequence, was cloned into the E1 deletion in Ad35 shuttle plasmid, pNEBAd35-2 (a precursor to the Ad35 ITR cassettes described above), generating pNEBAd35CMVgagBGHpA. pNEBAd35-2 contains Ad35 sequences from the left end of the genome (bp 4 to 456 and bp 3403 to 3886) with a unique SwaI site between bp 456 and 3403 at the position of the deletion. The gag expression cassette was obtained from a previously constructed shuttle plasmid by EcoRI digestion. Following the digestion the desired fragment was gel purified, treated with Klenow to obtain blunt ends and cloned into the SwaI site in pNEBAd35-2. This cloning step resulted in the gag expression cassette being inserted into the E1 deletion between bp 456 and 3403 in the E1 parallel orientation. The shuttle vector containing the gag transgene was digested to generate a DNA fragment consisting of the gag expression cassette flanked by Ad35 bp 4 to 456 and bp 3403 to 3886 and the fragment was purified after electrophoresis on an agarose gel. Cotransformation of BJ 5183 bacteria with the shuttle vector fragment and pAd34ΔE1ΔE4Ad5Orf6, linearized in the E1 region by digestion with Swa I, resulted in the generation of the Ad34 gag-containing pre-Adenovirus plasmid pAd34ΔE1gagΔE4Ad5Orf6 by homologous recombination. Potential clones were screened by restriction analysis.

A similar strategy was used to generate Ad34 pre-Ad plasmids containing a SEAP expression cassette. In this case a SEAP expression cassette consisting of: 1) the immediate early gene promoter from human cytomegalovirus, 2) the coding sequence of the human placental SEAP gene, and 3) the bovine growth hormone polyadenylation signal sequence was cloned into the E1 deletion in Ad35 shuttle plasmid, pNEBAd35-2, generating pNEBAd35CMVSEAPBGHpA. The SEAP expression cassette was obtained from a previously constructed shuttle plasmid by EcoRI digestion. Following the digestion the desired fragment was gel purified, treated with Klenow to obtain blunt ends and cloned into the SwaI site in pNEBAd35-2. The transgene was then recombined into the pAd34ΔE1ΔE4Ad5Orf6, generating pAd34ΔE1SEAPΔE4Ad5Orf6 as described above for the gag transgene.

All pre-Ad plasmids were rescued into virus and expanded to prepare CsCl purified stocks as described above.

### EXAMPLE 21

Construction of pMRKAd34ΔE1ΔE4Ad5Orf6

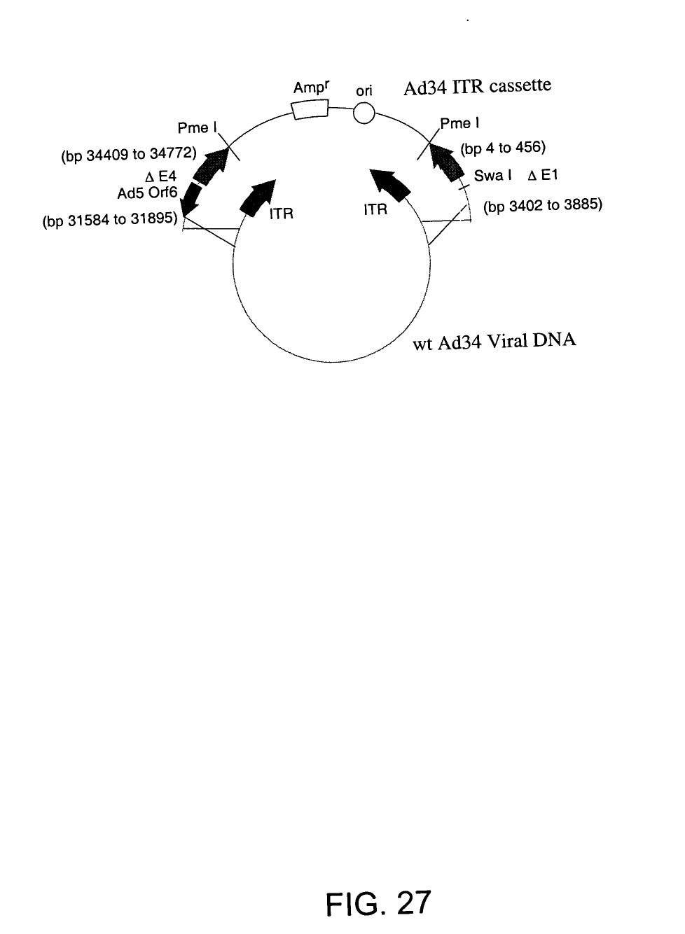

To construct an Ad34 pre-Ad plasmid that was composed entirely of Ad34 sequences, an Ad34 ITR cassette was generated. The Ad34 ITR cassette was constructed containing sequences from the right (bp 31584 to 31895 and bp 34409 to 34772) and left (bp 4 to 456 and bp 3402 to 3885) end of the Ad34 genome (see FIGS. 28A-1 to 28A-9) separated by plasmid sequences containing a bacterial origin of replication and an ampicillin resistance gene. These four segments were generated by PCR and cloned sequentially into pNEB 193, generating pNEBAd34-4. Next the Ad5 Orf6 open reading frame was generated by PCR and cloned between Ad34 bp 31895 and 34409 generating pNEBAd34-4Ad5Orf6 (the ITR cassette). PNEB193 is a commonly used commercially available cloning plasmid (New England Biolabs cat# N3051S) containing a bacterial origin of replication, ampicillin resistance gene and a multiple cloning site into which the PCR products were introduced. The ITR cassette contains a deletion of E1 sequences from Ad34 bp 457 to 3401 with a unique Swa I restriction site located in the deletion and an E4 deletion from Ad34 bp 31896 to 34408 into which Ad5 Orf6 was introduced in an E4 parallel orientation. In this construct Ad5Orf6 expression is driven by the Ad34 E4 promoter. The Ad34 sequences (bp 31584 to 31895 and bp 3402 to 3885) in the ITR cassette provided regions of homology with the purified Ad34 viral DNA in which bacterial recombination could occur following cotransformation into BJ 5183 bacteria (FIG. 27). The ITR cassette was also designed to contain unique restriction enzyme sites (PmeI) located at the end of the viral ITR's so that digestion would release the recombinant Ad34 genome from the plasmid sequences. Potential clones were screened by restriction analysis and one clone was selected as pMRKAd34ΔE1ΔE4Ad5Orf6.

### EXAMPLE 22

In Vivo Studies

A. Immunization

Cohorts of 3 rhesus macaques were given single intramuscular injections of one of the two vectors: (1) 10\hat{ }11 vp MRKAd5-SEAP (in MRKAd vector backbone disclosed in PCT/US01/28861, published Mar. 21, 2002); and (2) 10\hat{ }11 Vp Ad34ΔE1SEAPΔE4Ad5Orf6. Rhesus macaques were between 3-10 kg in weight. In all cases, the total dose of each vaccine was suspended in 1 mL of buffer. The macaques were anesthetized (ketamine/xylazine) and the vaccines were delivered i.m. in 0.5-mL aliquots into both deltoid muscles using tuberculin syringes (Becton-Dickinson, Franklin Lakes, N.J.). Peripheral blood mononuclear cells (PBMC) were prepared from blood samples collected at several time points during the immunization regimen. All animal care and treatment were in accordance with standards approved by the Institutional Animal Care and Use Committee according to the principles set forth in the Guide for Care and Use of Laboratory Animals, Institute of Laboratory Animal Resources, National Research Council.

B. SEAP Assay

Serum samples were analyzed for circulating human secreted alkaline phosphatase (SEAP) levels using TROPIX phospha-light chemiluminescent kit (Applied Biosystems Inc). Duplicate 5 μL aliquots of each serum were mixed with 45 μL of kit-supplied dilution buffer in a 96-well white DYNEX plate. Serially diluted solutions of a human placental alkaline phosphatase (Catalog no. M5905, Sigma, St. Louis, Mo.) in 10% naïve monkey serum served to provide the standard curve. Endogenous SEAP activity in the samples was inactivated by heating the well for 30 minutes at 65° C. Enzymatic SEAP activities in the samples were determined following the procedures described in the kit. Chemiluminescence readings (in relative light units) were recorded using DYNEX luminometer. RLU readings were converted to ng/mL SEAP using a log-log regression analyses.

C. ELISPOT Assay

The IFN-γ ELISPOT assays for rhesus macaques were conducted following a previously described protocol (Allen et al., 2001 J. Virol. 75(2):738-749), with some modifications. For antigen-specific stimulation, a peptide pool was prepared from 20-aa peptides that encompass the entire HIV-1 gag sequence with 10-aa overlaps (Synpep Corp., Dublin, Calif.). To each well, 50 μL of 2-4×10⁵ peripheral blood mononuclear cells (PBMCs) were added; the cells were counted using Beckman Coulter Z2 particle analyzer with a lower size cut-off set at 80 femtoliters (“fL”). Either 50 μL of media or the gag peptide pool at 8 pg/mL concentration per peptide was added to the PBMC. The samples were incubated at 37° C., 5% CO₂ for 20-24 hrs. Spots were developed accordingly and the plates were processed using custom-built imager and automatic counting subroutine based on the ImagePro platform (Silver Spring, Md.); the counts were normalized to 10⁶ cell input.

D. Intracellular Cytokine Staining (ICS)

To 1 ml of 2×10⁶ PBMC/mL in complete RPMI media (in 17×100 mm round bottom polypropylene tubes (Sarstedt, Newton, N.C.)), anti-hCD28 (clone L293, Becton-Dickinson) and anti-hCD49d (clone L25, Becton-Dickinson) monoclonal antibodies were added to a final concentration of 1 μg/mL. For gag-specific stimulation, 10 μL of the peptide pool (at 0.4 mg/mL per peptide) were added. The tubes were incubated at 37° C. for 1 hr., after which 20 μL of 5 mg/mL of brefeldin A (Sigma) were added. The cells were incubated for 16 hr at 37° C., 5% CO₂, 90% humidity. 4 mL cold PBS/2% FBS were added to each tube and the cells were pelleted for 10 min at 1200 rpm. The cells were re-suspended in PBS/2% FBS and stained (30 min, 4° C.) for surface markers using several fluorescent-tagged mAbs: 20 μL per tube anti-hCD3-APC, clone FN-18 (Biosource); 20 μL anti-hCD8-PerCP, clone SK1 (Becton Dickinson); and 20 μL anti-hCD4-PE, clone SK3 (Becton Dickinson). Sample handling from this stage was conducted in the dark. The cells were washed and incubated in 750 μL 1×FACS Perm buffer (Becton Dickinson) for 10 min at room temperature. The cells were pelleted and re-suspended in PBS/2% FBS and 0.1 μg of FITC-anti-hIFN-γ, clone MD-1 (Biosource) was added. After 30 min incubation, the cells were washed and re-suspended in PBS. Samples were analyzed using all four color channels of the Becton Dickinson FACSCalibur instrument. To analyze the data, the low side- and forward-scatter lymphocyte population was initially gated; a common fluorescence cut-off for cytokine-positive events was used for both CD4+ and CD8⁺ populations, and for both mock and gag-peptide reaction tubes of a sample.

E. Results

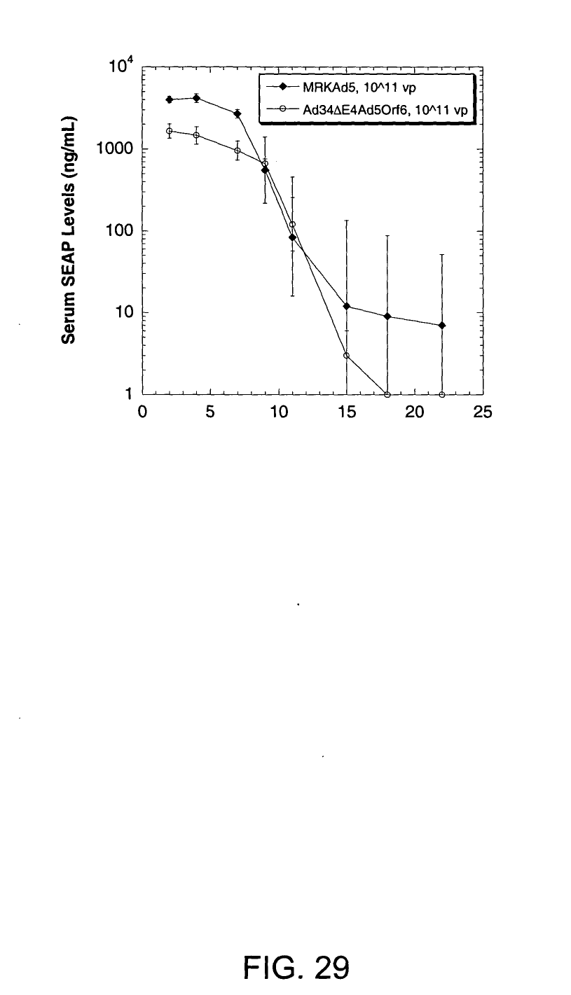

Expression: Serum samples prior to and after the injection were analyzed for circulating SEAP activities and the results are shown in FIG. 29. Results indicate that the peak levels of SEAP protein produced by the alternative adenovirus serotype were lower than but were within 3-fold of that of MRKAd5 at the same high dose level of 10\hat{ }11 vp (FIG. 29). The levels of SEAP in the serum dropped dramatically after day 10 and were close to background as early as day 15. These observations strongly indicate that the Ad34-based vector is efficient in expressing a transgene following intramuscular administration in a primate.

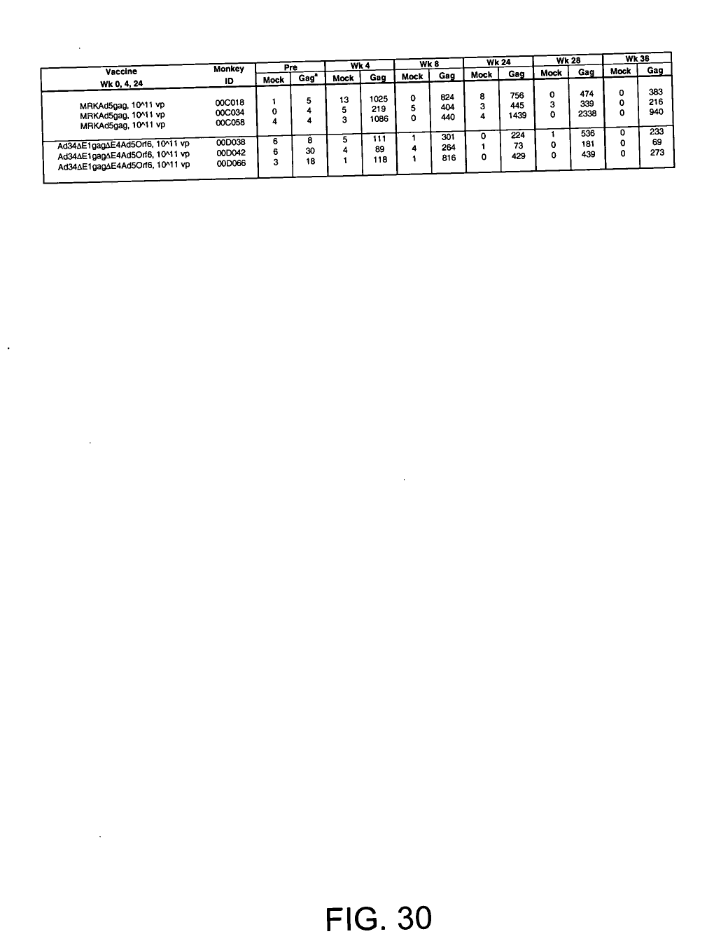

Immunogenicity: Vaccine-induced T cell responses against HIV-1 gag were quantified using IFN-gamma ELISPOT assay against a pool of 20-aa peptides that encompassed the entire protein sequence. The results are shown in FIG. 30; they are expressed as the number of spot-forming cells (SFC) per million peripheral blood mononuclear cells (PBMCs) that responded to the peptide pool or the mock (no peptide) control.

Immunization with gag-expressing Ad34 vector induced detectable levels of circulating gag-specific T cells immediately after a single dose of the vector. The responses improved following a second dose given at wk 4. Overall, the responses to the Ad34-based vector were slightly lower than those induced by the same dose of MRKAd5-gag. The results strongly indicate the Ad34-based vector can prime effectively for HIV-specific T cell responses.

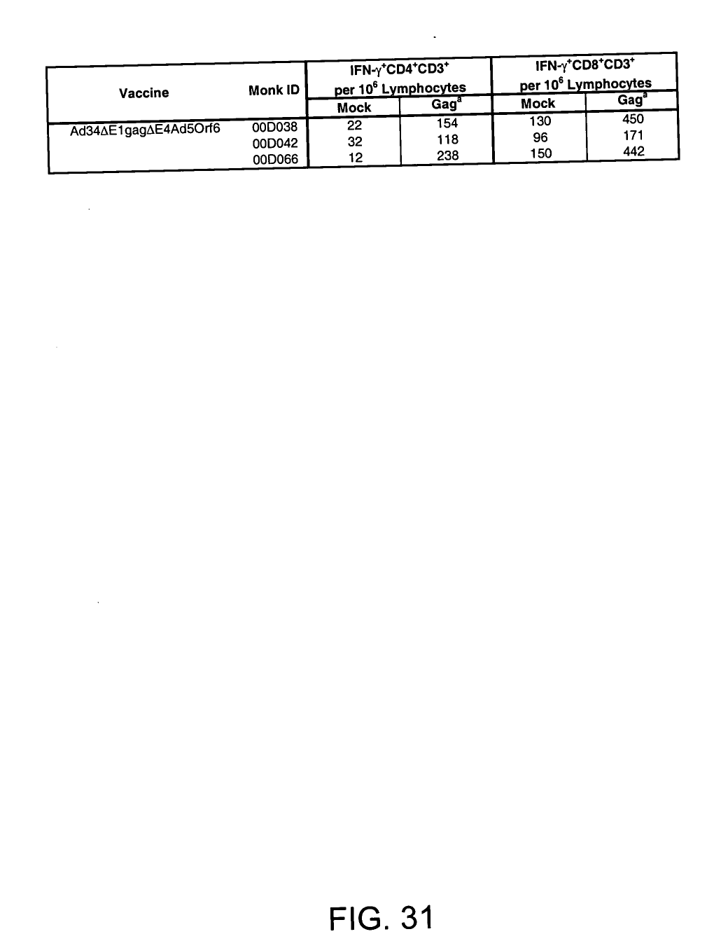

IFN-γ ICS analyses of the PBMC from the Ad34-immunized animals revealed that the vector can induce detectable levels of both CD4⁺ and CD8⁺ HIV-specific T cells (FIG. 31).

### EXAMPLE 23

Heterologous Immunization

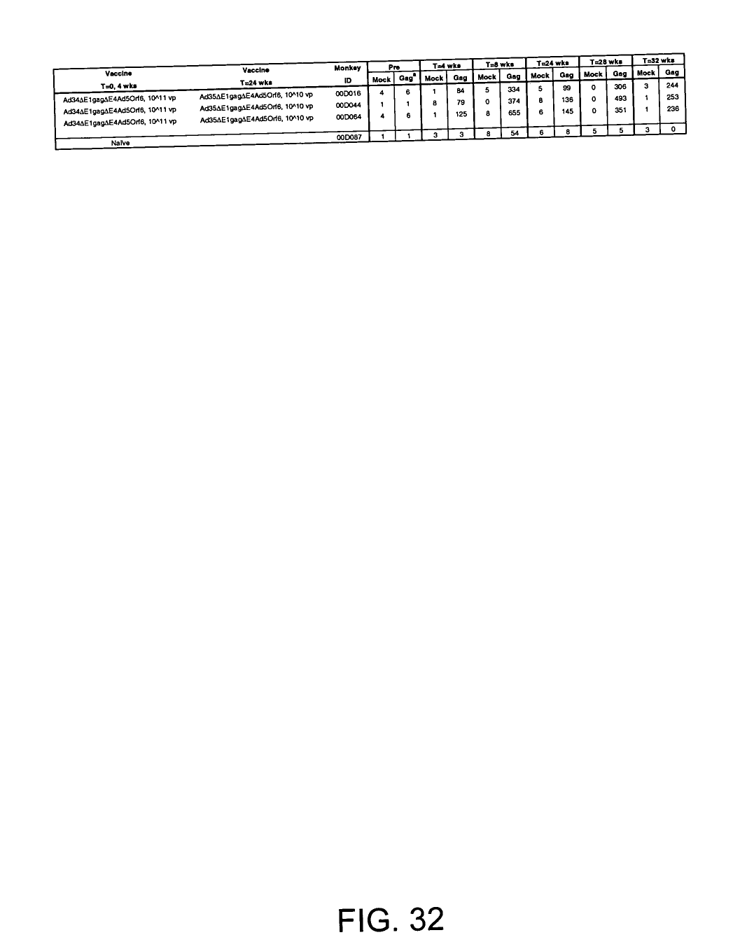

Cohorts of 3 monkeys were immunized (at wks 0, 4) with 10\hat{ }11 vp Ad34ΔE1gagΔE4Ad5Orf6 followed by a booster at week 24 with 10\hat{ }10 vp Ad35ΔE1gagΔE4Ad5Orf6. Vaccine-induced T cell responses against HIV-1 gag were quantified using IFN-gamma ELISPOT assay against a pool of 20-aa peptides that encompassed the entire protein sequence. The results are shown in FIG. 32; they are expressed as the number of spot-forming cells (SFC) per million peripheral blood mononuclear cells (PBMCs) that responded to the peptide pool or the mock (no peptide) control.

Immunization with gag-expressing Ad34 vector induced detectable levels of circulating gag-specific T cells that decreased to between 94-139 SFC/10\hat{ }6 PBMC at the time of the boost. Heterologous immunization with an Ad35-based HIV vector resulted in as much as a 3-fold increase in T cell responses.

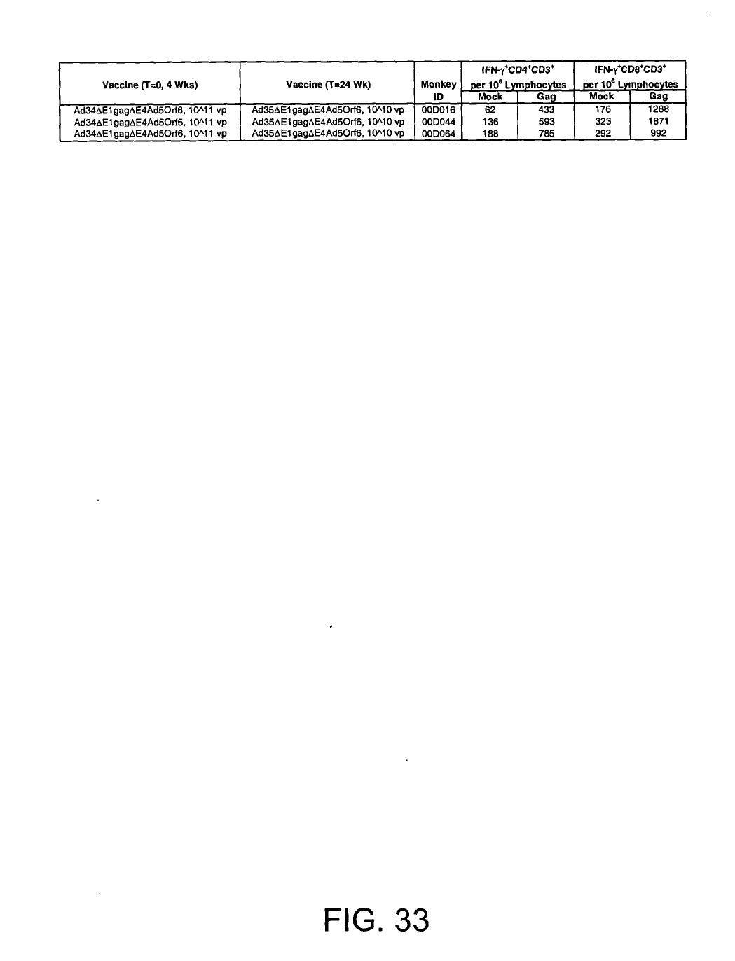

IFN-γ ICS analyses of the PBMCs from the Ad34 primed/Ad35 boosted animals at week 28 revealed that the vector can induce detectable levels of both CD4⁺ and CD8⁺ HIV-specific T cells (FIG. 33).

What is claimed is:

1. A means for propagating replication-defective adenovirus in an adenoviral E1-complementing cell line expressing E1 gene product(s) which are non-native to the adenovirus, which comprises:
(a) inserting all or a portion of a heterologous adenoviral E4 region comprising nucleic acid sequence encoding open reading frame 6 (ORF6) into a replication-defective adenovirus; wherein the E4 region or portion thereof inserted into the adenovirus is native to a virus of the same adenovirus serotype as the E1 gene product(s) expressed by the complementing cell line;
(b) introducing the replication-defective adenovirus into the adenoviral E1-complementing cell line;
(c) allowing the replication-defective adenovirus to propagate in the adenoviral E1-complementing cell line; and
(d) rescuing the propagated adenovirus.

2. A means in accordance with claim 1 wherein the heterologous adenoviral E4 region or portion thereof comprises the complete adenoviral E4-encoding region.

3. A means in accordance with claim 2 wherein the heterologous adenoviral E4 region or portion thereof comprises the complete adenoviral E4-encoding region and native E4 promoter.

4. A means in accordance with claim 1 wherein the heterologous adenoviral E4 region or portion thereof is inserted into the replication-defective virus in place of nucleic acid sequence encoding open reading frame 6 (ORF6).

5. A means in accordance with claim 1 wherein the heterologous adenoviral E4 region or portion thereof is inserted into the replication-defective virus in place of nucleic acid sequence encoding the complete adenoviral E4-encoding region.

6. A means in accordance with claim 1 wherein the heterologous adenoviral E4 region or portion thereof is derived from a subgroup C adenovirus.

7. A means in accordance with claim 1 wherein the subgroup C adenovirus is adenovirus of serotype 5.

8. A means in accordance with claim 7 wherein the replication-defective adenovirus is an adenovirus of subgroup B.

9. A means in accordance with claim 7 wherein the replication-defective adenovirus is an adenovirus of serotype 35.

10. A means in accordance with claim 1 wherein the heterologous adenoviral E4 region or portion thereof is operatively linked to a heterologous promoter.

11. A means in accordance with claim 1 wherein the adenoviral E1-complementing cell line is a PER.C6® cell line.

12. A replication-defective adenovirus comprising all or a portion of a heterologous E4 region comprising a heterologous adenoviral open reading frame 6 (ORF6).

13. A replication-defective adenovirus in accordance with claim 12 wherein the adenovirus comprises a heterologous gene of interest.

14. A replication-defective adenovirus in accordance with claim 13 wherein the heterologous gene of interest is a gene encoding an HIV-1 antigen.

15. A replication-defective adenovirus in accordance with claim 14 wherein the HIV-1 antigen is selected from the group consisting of HIV-1 gag, pol, nef and env.

16. A replication-defective adenovirus comprising all or a portion of a heterologous E4 region comprising a heterologous adenoviral open reading frame 6 (ORF6) and a gene encoding HIV-1 gag.

17. A replication-defective adenovirus comprising all or a portion of a heterologous E4 region comprising a heterologous adenoviral open reading frame 6 (ORF6) in place of a native E4 region or portion thereof comprising ORF6.

18. A replication-defective adenovirus comprising all or a portion of a heterologous E4 region comprising a complete heterologous E4 region in place of a complete native E4 region.

19. A replication-defective adenovirus comprising a heterologous E4 region or portion thereof comprising a complete heterologous E4 region including E4 promoter in place of a complete native E4 region.

20. Adenovirus propagated in accordance with the means of claim 1.

21. A means in accordance with claim 1 wherein the replication-defective adenovirus comprises a heterologous gene of interest.

22. A means in accordance with claim 21 wherein the heterologous gene of interest is a gene encoding an HIV-1 antigen.

23. A means in accordance with claim 22 wherein the HIV-1 antigen is selected from the group consisting of: HIV-1 gag, pol, nef and env.

24. A replication-defective adenovirus of serotype 35 comprising all or a portion of an adenovirus serotype 5 E4 region comprising open reading frame 6 (ORF6) and a heterologous gene of interest.

25. A replication-defective adenovirus in accordance with claim 24 wherein the heterologous gene of interest is a gene encoding an HIV-1 antigen.

26. A replication-defective adenovirus in accordance with claim 25 wherein the HIV-1 antigen is selected from the group consisting of: HIV-1 gag, pol, nef and env.

27. A replication-defective adenovirus of serotype 35 comprising all or a portion of an adenovirus serotype 5 E4 region comprising open reading frame 6 (ORF6) and a gene encoding HIV-1 gag.

28. A recombinant adenoviral vector of serotype 24 which comprises an E4 gene or a segment of an E4 gene comprising open reading frame 6 (“ORF6”) of an alternative serotype.

29. A population of cells comprising the recombinant adenoviral vector of claim 28.

30. A method for producing recombinant, replication-defective adenovirus particles comprising:
(a) introducing a recombinant adenoviral vector of claim 28 into a population of cells expressing adenovirus E1; and
(b) harvesting the resultant recombinant, replication-defective adenovirus.

31. Purified recombinant, replication-defective adenovirus particles harvested in accordance with the method of claim 30.

32. A composition comprising purified recombinant adenovirus particles in accordance with claim 31.

33. A composition in accordance with claim 32 which comprises a physiologically acceptable carrier.

34. A recombinant adenoviral vector in accordance with claim 28 which is at least partially deleted in E1 and devoid of E1 activity and comprises a heterologous nucleic acid.

35. A composition comprising purified recombinant adenoviral particles in accordance with claim 31 which are at least partially deleted in E1 and devoid of E1 activity and comprise a heterologous nucleic acid.

36. A method for effecting the delivery and expression of heterologous nucleic acid comprising administering the composition of claim 35 prior or subsequent to administration of the heterologous nucleic acid with the same or different vector.

37. A method in accordance with claim 36 wherein the composition is preceded or followed by administration of heterologous nucleic acid with an adenovirus of a different serotype.

38. A composition in accordance with claim 35 wherein the heterologous nucleic acid encodes an HIV antigen.

39. A method for generating a cellular-mediated immune response against HIV in an individual comprising administering to the individual a composition of claim 38.

40. A composition in accordance with claim 39 wherein the HIV antigen is HIV-1 gag or immunologically relevant modification thereof.

41. A composition in accordance with claim 39 wherein the HIV antigen is HIV-1 nef or immunologically relevant modification thereof.

42. A composition in accordance with claim 39 wherein the HIV antigen is HIV-1 pol or immunologically relevant modification thereof.

43. A recombinant adenoviral vector of serotype 24 which is at least partially deleted in E1 and devoid of E1 activity; wherein said vector comprises an E4 gene or a segment of an E4 gene from adenovirus serotype 5 comprising open reading frame 6 (“ORF6”), and a heterologous nucleic acid.

44. A population of cells comprising the recombinant adenoviral vector of claim 43.

45. A method for producing recombinant, replication-defective adenovirus particles comprising:
(a) introducing a recombinant adenoviral vector of claim 43 into a population of cells expressing adenovirus serotype 5 E1; and
(b) harvesting the resultant recombinant, replication-defective adenovirus.

46. Purified recombinant, replication-defective adenovirus particles harvested in accordance with the method of claim 45.

47. A composition comprising purified recombinant adenovirus particles in accordance with claim 46.

48. A composition in accordance with claim 47 which comprises a physiologically acceptable carrier.

49. A method for effecting the delivery and expression of the heterologous nucleic acid comprising administering the composition of claim 48 prior or subsequent to administration of the heterologous nucleic acid with the same or different vector.

50. A method in accordance with claim 49 above wherein the composition is preceded or followed by administration of the heterologous nucleic acid with an adenovirus of a different serotype.

51. A composition in accordance with claim 48 wherein the heterologous nucleic acid encodes an HIV antigen.

52. A method for generating a cellular-mediated immune response against HIV in an individual comprising administering to the individual a composition of claim 51.

53. A composition in accordance with claim 51 wherein the HIV antigen is HIV-1 gag or immunologically relevant modification thereof.

54. A composition in accordance with claim 51 wherein the HIV antigen is HIV-1 nef or immunologically relevant modification thereof.

55. A composition in accordance with claim 51 wherein the HIV antigen is HIV-1 pol or immunologically relevant modification thereof.

56 A recombinant adenoviral vector of serotype 34 which comprises an E4 gene or a segment of an E4 gene comprising open reading frame 6 (“ORF6”) of an alternative serotype.

57. A population of cells comprising the recombinant adenoviral vector of claim 56.

58. A method for producing recombinant, replication-defective adenovirus particles comprising:
(a) introducing a recombinant adenoviral vector of claim 56 into a population of cells expressing adenovirus E1; and
(b) harvesting the resultant recombinant, replication-defective adenovirus.

59. Purified recombinant, replication-defective adenovirus particles harvested in accordance with the method of claim 58.

60. A composition comprising purified recombinant adenovirus particles in accordance with claim 59.

61. A composition in accordance with claim 60 which comprises a physiologically acceptable carrier.

62. A recombinant adenoviral vector in accordance with claim 56 which is at least partially deleted in E1 and devoid of E1 activity and comprises a heterologous nucleic acid.

63. A composition comprising purified recombinant adenoviral particles in accordance with claim 59 which are at least partially deleted in E1 and devoid of E1 activity and comprise a heterologous nucleic acid.

64. A method for effecting the delivery and expression of heterologous nucleic acid comprising administering the composition of claim 63 prior or subsequent to administration of the heterologous nucleic acid with the same or different vector.

65. A method in accordance with claim 64 wherein the composition is preceded or followed by administration of heterologous nucleic acid with an adenovirus of a different serotype.

66. A composition in accordance with claim 63 wherein the heterologous nucleic acid encodes an HIV antigen.

67. A method for generating a cellular-mediated immune response against HIV in an individual comprising administering to the individual a composition of claim 66.

68. A composition in accordance with claim 67 wherein the HIV antigen is HIV-1 gag or immunologically relevant modification thereof.

69. A composition in accordance with claim 67 wherein the HIV antigen is HIV-1 nef or immunologically relevant modification thereof.

70. A composition in accordance with claim 67 wherein the HIV antigen is HIV-1 pol or immunologically relevant modification thereof.

71. A recombinant adenoviral vector of serotype 34 which is at least partially deleted in E1 and devoid of E1 activity; wherein said vector comprises an E4 gene or a segment of an E4 gene from adenovirus serotype 5 comprising open reading frame 6 (“ORF6”), and a heterologous nucleic acid.

72. A population of cells comprising the recombinant adenoviral vector of claim 71.

73. A method for producing recombinant, replication-defective adenovirus particles comprising:
(a) introducing a recombinant adenoviral vector of claim 71 into a population of cells expressing adenovirus serotype 5 E1; and
(b) harvesting the resultant recombinant, replication-defective adenovirus.

74. Purified recombinant, replication-defective adenovirus particles harvested in accordance with the method of claim 73.

75. A composition comprising purified recombinant adenovirus particles in accordance with claim 74.

76. A composition in accordance with claim 75 which comprises a physiologically acceptable carrier.

77. A method for effecting the delivery and expression of the heterologous nucleic acid comprising administering the composition of claim 76 prior or subsequent to administration of the heterologous nucleic acid with the same or different vector.

78. A method in accordance with claim 77 above wherein the composition is preceded or followed by administration of the heterologous nucleic acid with an adenovirus of a different serotype.

79. A composition in accordance with claim 76 wherein the heterologous nucleic acid encodes an HIV antigen.

80. A method for generating a cellular-mediated immune response against HIV in an individual comprising administering to the individual a composition of claim 79.

81. A composition in accordance with claim 79 wherein the HIV antigen is HIV-1 gag or immunologically relevant modification thereof.

82. A composition in accordance with claim 79 wherein the HIV antigen is HIV-1 nef or immunologically relevant modification thereof.

83. A composition in accordance with claim 79 wherein the HIV antigen is HIV-1 pol or immunologically relevant modification thereof.
